SEQUENCE LISTING (4 sequences)

SEQ ID NO: 1 - protein, 265 aa, Homo sapiens
MLLWPLRGWAARALRCFGPGSRGSPASGPGPRRVQRRAWPPDKEQEKEKKSVICVEGNIASGKTTCLEFFSNATDVEVLTEPVSKWRNVRGHNPLGLMYHDASRWGLTLQTYVQLTMLDRHTRPQVSSVRLMERSIHSARYIFVENLYRSGKMPEVDYVVLSEWFDWILRNMDVSVDLIVYLRTNPETCYQRLKKRCREEEKVIPLEYLEAIHHLHEEWLIKGSLFPMAAPVLVIEADHHMERMLELFEQNRDRILTPENRKHCP

SEQ ID NO: 2 - DNA, 795 bp, Homo sapiens
atgctgctgtggccgctgcggggctgggccgcccgggcgctgcgctgctttgggccgggaagtcgcgggagcccggcctcaggccccgggccgcggagggtgcagcgccgggcctggcctcccgataaagaacaggaaaaagagaaaaaatcagtgatctgtgtcgagggcaatattgcaagtgggaagacgacatgcctggaattcttctccaacgcgacagacgtcgaggtgttaacggagcctgtgtccaagtggagaaatgtccgtggccacaatcctctgggcctgatgtaccacgatgcctctcgctggggtcttacgctacagacttatgtgcagctcaccatgctggacaggcatactcgtcctcaggtgtcatctgtacggttgatggagaggtcgattcacagcgcaagatacatttttgtagaaaacctgtatagaagtgggaagatgccagaagtggactatgtagttctgtcggaatggtttgactggatcttgaggaacatggacgtgtctgttgatttgatagtttaccttcggaccaatcctgagacttgttaccagaggttaaagaagagatgcagggaagaggagaaggtcattccgctggaatacctggaagcaattcaccatctccatgaggagtggctcatcaaaggcagccttttccccatggcagcccctgttctggtgattgaggctgaccaccacatggagaggatgttagaactctttgaacaaaatcgggatcgaatattaactccagagaatcggaagcattgccca

SEQ ID NO: 3 - protein, 216 aa, Homo sapiens
MSVICVEGNIASGKTTCLEFFSNATDVEVLTEPVSKWRNVRGHDPLGLMYHDASRWGLTLQTYVQLTMLDRHTRPQVSSVRLMERSIHSARYIFVENLYRSGKMPEVDYVVLSEWFDWILRNMDVSVDLIVYLRTNPETCYQRLKKRCREEEKVIPLEYLEAIHHLHEEWLIKGSLFPMAAPVLVIEADHHMERMLELFEQNRDRILTPENRKHCP

SEQ ID NO: 4 - protein, 216 aa, Homo sapiens
MSVICVEGNIASGKTTCLEFFSNATDVEVLTEPVSKWRNVRGHDPLGLMYHDASRWGLTFQTYVQLTMLDRHTRPQVSSVRLMERSIHSARYIFVENLYRSGKMPEVDYVVLSEWFDWILRNMDVSVDLIVYLRTNPETCYQRLKKRCREEEKVIPLEYLEAIHHLHEEWLIKGSLFPMAAPVLVIEADHHMERMLELFEQNRDRILTPENRKHCP

(19) United States
(12) Patent Application Publication
(10) Pub. No.: US 2014/0248212 A1
(43) Pub. Date: Sep. 4, 2014

(54) NON-IMMUNOGENIC POSITRON EMISSION TOMOGRAPHY REPORTER GENE SYSTEMS

(71) Applicants: Caius G. Radu, Los Angeles, CA (US); Johannes Czernin, Pacific Palisades, CA (US); Dean O. Campbell, Los Angeles, CA (US); Shahriar S. Yaghoubi, Los Angeles, CA (US); Nagichettiar Satyamurthy, Los Angeles, CA (US); Arnon Lavie, Chicago, IL (US)

(72) Inventors: Caius G. Radu, Los Angeles, CA (US); Johannes Czernin, Pacific Palisades, CA (US); Dean O. Campbell, Los Angeles, CA (US); Shahriar S. Yaghoubi, Los Angeles, CA (US); Nagichettiar Satyamurthy, Los Angeles, CA (US); Arnon Lavie, Chicago, IL (US)

(73) Assignee: THE REGENTS OF THE UNIVERSITY OF CALIFORNIA, Oakland, CA (US)

(21) Appl. No.: 14/354,770

(22) PCT Filed: Aug. 6, 2012
(86) PCT No.: PCT/US2012/049753

§ 371 Date: Apr. 28, 2014

Related U.S. Application Data
(60) Provisional application No. 61/515,743, filed on Aug. 5, 2011.

(51) Int. Cl. A61K 51/04; C12Q 1/48; C12N 9/12

(52) U.S. Cl. CPC A61K 51/0491; C12N 9/1211; C12Q 1/485
USPC 424/1.73; 435/194; 536/23.2; 435/320.1; 435/15

(57) ABSTRACT

Embodiments of the invention include a PET/SPECT reporter gene system that uses enhanced non-immunogenic versions of a human mitochondrial thymidine kinase 2 expressed in cytoplasm to preferentially trap novel PET/SPECT radiolabeled L and D-enantiomer analogs of the natural substrate thymidine. Such highly sensitive, non-immunogenic reporter genes function in combination with a set of novel, radiolabeled probes in whole body molecular imaging applications using positron emission tomography (PET) or single photon emission computed tomography (SPECT).

### REFERENCE TO RELATED APPLICATIONS

This application claims priority under Section 119(e) from U.S. Provisional Application Ser. No. 61/515,743, filed Aug. 5, 2011, the contents of which are incorporated herein by reference.

### STATEMENT OF GOVERNMENT SUPPORT

This invention was made with Government support of Grant No. CA160770-02 awarded by the National Institutes of Health. The Government has certain rights in this invention.

### BACKGROUND OF THE INVENTION

1. Field of the Invention

The present invention relates to systems, compositions of matter, and techniques for the specific identification and tracking of genes and cells. In particular, the invention relates to positron emission tomography (PET) and single photon emission computed tomography (SPECT) reporter genes and reporter probe systems.

2. Description of Related Art

Gene and cell-based therapies hold great promise in oncology and many other areas of medicine. In such technologies, targeted therapeutic cells (TCs) or therapeutic transgenes (TGs) can be injected into patients to restore normal organ function in degenerative diseases, eliminate cancer cells, or correct a system malfunction in other diseases. In such contexts, the selection of appropriate cells, optimization of the cells (e.g., in vitro genetic engineering) to perform a specific therapeutic function, and determination of an appropriate administration route and cell dose are necessary to achieve the desired cell therapeutic effect. Similarly, identifying the appropriate transgenes and determining an optimal delivery of the transgenes to target tissues are necessary to achieve the desired gene therapeutic effect. However, achieving these objectives remains a major challenge. Despite decades of research in gene- and cell-based therapies, there are currently no approved products for routine oncological applications in the United States.

A formidable roadblock in this technology is an inability to routinely monitor the tissue pharmacokinetics (PK) of therapeutic genes and cells and correlate this information with therapeutic outcomes. Most cell/gene therapy trials use invasive biopsy techniques to localize TGs or TCs at target sites. This is a significant problem since tissue biopsies are prone to sampling errors, cannot reveal either whole body therapeutic gene or cell distribution at any one time or alterations in distribution with time. Moreover, biopsies are invasive procedures that may put patients at risk. Inappropriate administration of TCs or TGs based on incomplete and/or unreliable tissue PK data may not yield good treatment efficacy and may lead to severe adverse effects, up to and including lethality (see, e.g. Morgan, R. A., et al. Molecular Therapy (2010) 18 4, 843-851). Thus there is an unmet need for techniques to monitor the whole-body tissue distribution of TCs and TGs—to quantify TCs and to measure TG expression at all locations, non-invasively and sequentially following treatment.

Unmet needs in this technology are reflected, for example, in the hurdles encountered in clinical trials of Adoptive Cellular Gene Therapy (ACGT) against melanoma. ACGT is a cancer immunotherapy technique under evaluation in multiple FDA-approved clinical trials. In ACGT, billions of patient-derived T cell receptor (TCR) transgenic cytotoxic T lymphocytes (CTLs) generated ex vivo are transplanted back into the patient (the immunological term for T cell transplantation is “adoptive transfer”). Adoptively transferred T cells proliferate, seek out and kill tumor cells (see, e.g. Rosenberg, S. A., et al. Nat Rev Cancer 8:299-308, 2008; FIG. 2A). Building on the pioneering work by Rosenberg and colleagues (see, e.g. Rosenberg, S. A., et al. N Engl J Med 359:1072, 2008), studies have shown that ACGT induces partial responses in patients with advanced melanoma. Unfortunately, these responses are not sustained and most patients relapsed within 6 months. FIG. 2B shows a representative clinical case from one ACGT trial. Sequential [¹⁸F]FDG PET scans showed the remarkable anti-tumor activity of ACGT (note the regression of many melanoma lesions), but also identified a resistant lesion. A biopsy of this resistant lesion revealed low numbers of tumor infiltrating therapeutic T cells, presumably because cancer cells at this site lacked the expression of the target antigen. Such findings show that an imaging procedure to detect and quantify therapeutic T cells in melanoma lesions throughout the body is needed to monitor and optimize immune cell-based therapies against melanoma and, by extrapolation, against other cancers.

An alternative to biopsies is a non-invasive, repeated, quantifiable imaging of therapeutic genes and cells throughout the bodies of living subjects. One such alternative is the use of PET reporter gene (PRG) imaging, which provides a possible solution to the tissue PK measurements problem affecting gene and cell-based therapies. A PRG encodes a protein that mediates the specific cellular accumulation of a PET reporter probe (PRP) labeled with a positron-emitting isotope. Several types of PET reporter gene/probe (PRG-PRP) systems have been developed (see, e.g. FIG. 3 and Min, J. J., et al. Handb Exp Pharmacol, 277-303, 2008). PRG imaging can enable serial, quantitative, and sensitive detection of gene modified therapeutic T cells (and other gene and cell based therapies) in vivo.

In a typical PET/SPECT reporter gene embodiment, a foreign gene is introduced into cells of interest; the activity of the reporter gene leads to preferential cellular accumulation of a radioactive probe. Cells engineered to express the PET/SPECT reporter gene are in this way “tagged” and their presence can be detected and quantified at various locations within the body using PET or SPECT. Alternatively, PET/SPECT reporter gene systems can be used to study gene expression in vivo. This is accomplished by placing the reporter gene downstream from a promoter or regulatory region of interest. While numerous PET/SPECT reporter gene systems have been described in recent years, most of them are based on one of the following mechanisms: (i) enzymatic modification of the PET/SPECT probe followed by intracellular trapping; (ii) accumulation via plasma membrane transport mechanisms; and, (iii), cell surface receptor mapping using radioactive ligands or monoclonal antibody fragments. A brief summary of conventional PET reporter gene systems and probes is presented in FIG. 22. Further description of PET/SPECT reporter gene technologies are covered, for example, in Gambhir, S. S, and S. S. Yaghoubi, Eds. (2010), Molecular Imaging With Reporter Genes, Cambridge Molecular Imaging, Cambridge University Press.

Various molecular imaging groups have worked to develop PRG-PRP techniques for non-invasively, repeatedly and quantitatively measuring gene expression in living animals. One of the first studies developed Dopamine type 2 receptor (D₂R) as a PRG, and 3-(2′[¹⁸F]fluoroethyl)spiperone (FESP)—a ligand that binds to the D₂R—as the PRP (see, e.g. FIG. 3 and MacLaren, D. C., et al. Gene Ther 6:785-791, 1999). To convert the D₂R system to a reporter with no biological activity, a mutant D₂R gene (D₂R80A) in which ligand binding is uncoupled from signal transduction was developed as a “second generation” D₂R PRG (see, e.g. Liang, Q., et al. Gene Ther 8:1490-1498, 2001). The D₂R80A PRG was noted to work well when expressed from strong promoters (see, e.g. Gambhir, S. S., et al. J Nucl Med 39:2003-2011, 1998). However, its sensitivity was inadequate for many imaging applications. Because receptor-ligand PRG-PRP systems are stoichiometric, catalytic enzyme-substrate PRG-PRP systems would be preferred.

Herpes Simplex Virus type 1 thymidine kinase (HSV1-tk) has also been developed as a catalytic PRG and ¹⁸F-labeled fluoroganciclovir (FGCV) as a PRP (see, e.g. Gambhir, S. S., et al. J Nucl Med 39:2003-2011, 1998). Similar studies are known in the art (see, e.g. Tjuvajev, J. G., et al. Cancer Res 58:4333-4341, 1998). To increase sensitivity, researchers searched for “second generation” HSV1-tk reporter genes that utilized acycloguanosines more effectively, and thymidine (the “natural” substrate) less effectively, and identified HSV1-sr39tk as a substantially more sensitive PRG (see, e.g. Gambhir, S. S., et al. Proc Natl Acad Sci USA 97:2785-2790, 2000). To improve the PRG-PRP system further, researchers tested a series of ¹⁸F-labelled acycloguanosines and determined their relative efficacies as PRPs with HSV1-sr39tk (see, e.g. Min, J. J., et al. Eur J Nucl Med Mol Imaging 30:1547-1560, 2003; Yaghoubi, S., et al. J Nucl Med 42:1225-1234, 2001). 9-[(4-[¹⁸F]fluoro-3-hydroxymethylbutyl)guanine (FHBG) was identified in these studies. Human FHBG PK and dosimetry studies have been conducted in preparation for clinical trials (see, e.g. Yaghoubi, S., et al. J Nucl Med 42:1225-1234, 2001), to participate in comparative quantification of the D₂R80A and HSV1-sr39tk reporter systems (see, e.g. Yaghoubi, S. S., et al. Gene Ther 8:1072-1080, 2001), and to use FHBG to monitor the progress of HSV1-sr39tk/ganciclovir suicide gene therapy in cancer (see, e.g. Yaghoubi, S. S., et al. Cancer Gene Ther 12:329-339, 2005). The development of “second generation” PRG-PRPs for the HSV1-tk and D₂R systems demonstrates an early and continued commitment to optimizing PRG-PRP pairings, for experimental and clinical applications.

The HSV1-sr39tk/FHBG system is the current standard of comparison in evaluating new PRG-PRP combinations. The gold standard for PRG imaging is the viral HSV1-tk and sr39tk, its optimized analog (see, e.g. Min, J. J., et al. Handb Exp Pharmacol, 277-303, 2008). The advantages of HSV1-tk over other PRGs are its high sensitivity and dual function as a suicide/safety gene. However, its main disadvantage is immunogenicity, due to the very low sequence homology (˜10%, see, e.g. Eriksson, S., et al. Cell Mol Life Sci 59, 1327-1346, 2002) between the viral protein and human nucleoside kinases. FIG. 5 illustrates the molecular and cellular mechanisms of HSV1-tk immunogenicity and the potential detrimental effects of immunogenicity on treatment efficacy. The immunogenicity of HSV1-tk has been documented in allogeneic hematopoietic stem cell transplants in leukemic patients (see, e.g. Riddell, S. R., et al. Nat Med 2:216-223, 1996; Bonini, C., et al. Science 276:1719-1724, 1997; Tiberghien, P., et al. Blood 97:63-72, 2001). A significant incidence of immune responses occurs against HSV1-tk, accompanied by a decrease in the number of circulating therapeutic cells (see, e.g. Bonini, C., et al. Science 276:1719-1724, 1997; Verzeletti, S., et al. Hum Gene Ther 9:2243-2251, 1998). Even more substantial immune responses were reported following infusion of HSV1-tk modified donor T cells in immunocompetent patients (see, e.g. Berger, C., et al. Blood 107:2294-2302, 2006).

One solution to the immunogenicity problem is to replace the viral PRG with human PRGs. Several human-gene derived PRGs have been investigated including D₂R (see, e.g. Liang, Q., et al. Gene Ther 8:1490-1498, 2001), human somatostatin receptor 2 (hSSR2) (see, e.g. Rogers, B. E., et al. J Nucl Med 46:1889-1897, 2005), sodium-iodide symporter (NIS) (see, e.g. Che, J., et al. Mol Imaging 4:128-136, 2005), human norepinephrine transporter (hNET) (see, e.g. Buursma, A. R., et al. J Nucl Med 46:2068-2075, 2005; Moroz, M. A., et al. J Nucl Med 48:827-836, 2007), truncated human thymidine kinase 2 (hTK2) (see, e.g. Ponomarev, V., et al. J Nucl Med 48:819-826, 2007), and human deoxycytidine kinase (dCK) (see, e.g. Likar, Y., et al. J Nucl Med 51:1395-1403, 2010). Since direct comparisons are lacking, it is not known how hSSR2, D₂R, and NIS compare to HSV1-sr39tk. The un-mutated hTK2 PRG is significantly less sensitive than sr39tk (see, e.g. Ponomarev, V., et al. J Nucl Med 48:819-826, 2007). hNET has comparable or slightly lower sensitivity than HSV1-tk, with both PRGs enabling detection by microPET of as few as 10⁴ CTLs injected into tumor xenografts (see, e.g. Doubrovin, M. M., et al. Cancer Res 67:11959-11969, 2007). However, using the hNET/[¹²⁴I]MIBG system for clinical imaging may require the development of ¹⁸F-labeled probes, since ¹²⁴I has a long half-life and patients may be exposed to high doses of radiation.

For the reasons noted above, there is a need in the art for novel PET reporter gene/reporter probe systems. Embodiments of the invention disclosed herein meet this as well as other needs.

### SUMMARY OF THE INVENTION

Embodiments of the present invention provide systems, materials, and methods for non-invasive imaging of gene expression. Embodiments of the invention can be used, for example, in monitoring the kinetics of therapeutic genes and cells in vivo using positron emission tomography (PET) or single photon emission computed tomography (SPECT). Such PET technologies are useful in quantitative, non-invasive molecular imaging, a methodological approach the applicable in a variety of preclinical and clinical settings. Illustrative embodiments of the invention include a highly sensitive, non-immunogenic reporter gene that can function with a set of novel, radiolabeled probes in whole body molecular imaging applications using positron emission tomography or single photon emission computed tomography.

Embodiments of the invention have a variety of applications. For example, embedding PET reporter gene imaging in clinical trials of cell and gene therapies can be used to prevent “blinded” attempts to optimize these therapies. Embodiments of the invention also provide the means to detect the occurrence of adverse physiological phenomena, such as malignant transformation of cells and autoimmune-mediated destruction of normal tissues. The PET reporter gene (PRG) based imaging kits described herein further have the benefit of encouraging those of skill in cellular and gene therapy technologies to apply long-term non-invasive molecular imaging to pharmacokinetic studies.

As discussed below, embodiments of the invention disclosed herein can addresses key limitations of current PRG technologies. In particular, instead of the commonly used highly immunogenic viral proteins of conventional PRGs, a number novel PET reporters based on fully human proteins are disclosed herein. The use of human genes in embodiments of the invention significantly reduces the probability of patients developing immunity against therapeutic cells (and other delivery systems) engineered to express PRGs, one of the most significant challenges to the clinical implementation of current PRGs. The disclosure further describes the effect of reporter gene expression on nucleotide pools.

In typical embodiments of the invention, a PET/SPECT reporter gene uses an enhanced version of human mitochondrial thymidine kinase 2 (htk2) expressed in cytoplasm to preferentially trap novel PET/SPECT radiolabeled L and D-enantiomer analogs of thymidine, a natural substrate. Endogenous hTK2 enzyme provides the first phosphorylation step in the salvage pathway of deoxyribonucleosides. In one exemplary embodiment, the enhanced htk2 is not immunogenic in humans. In addition, the enhanced htk2 reporter gene can serve as a safety gene to destroy cells expressing it when they malfunction. A method is also provided for mutating the htk2 gene to enhance the catalytic activity of its enzyme product for the phosphorylation of several thymidine analogs or to decrease the catalytic activity of its enzyme product for the phosphorylation of D-enantiomer thymidine.

The invention disclosed herein has a number of embodiments. One embodiment of the invention comprises a human thymidine kinase 2 polypeptide comprising amino acid residues 51-265 of SEQ ID NO: 1, wherein the thymidine kinase polypeptide comprises an amino acid substitution at amino acid residue position 93 and/or amino acid residue position 109 of SEQ ID NO: 1. In typical embodiment, the thymidine kinase polypeptide is designed or selected to exhibit a certain functional activity, for example a decreased susceptibility to thymidine triphosphate mediated feedback inhibition as compared to wild type polypeptide shown in SEQ ID NO: 1, an ability to phosphorylate 2′-deoxy-2′-18^{F}-5-methyl-1-β-L-arabinofuranosyluracil, or the like. In typical embodiments of the invention, the thymidine kinase polypeptide does not include the first 50 amino acids of SEQ ID NO: 1 and comprises an amino acid substitution at amino acid residue position 93 of SEQ ID NO: 1 (e.g. N93D) and an amino acid substitution at amino acid residue position 109 of SEQ ID NO: 1 (e.g. L109M or L109F). Optionally, the polypeptide comprises a set of amino acid mutations such as a polypeptide huΔ₁₋₅₀TK2-N93D/L109M (see, e.g. the embodiments of huTK2 shown in FIG. 21).

Other embodiments of the invention include a nucleic acid molecule comprising DNA encoding a human thymidine kinase 2 polypeptide comprising amino acid residues 51-265 of SEQ ID NO: 1. Typically in these embodiments, the thymidine kinase polypeptide comprises an amino acid substitution at amino acid residue position 93 and/or amino acid residue position 109 of SEQ ID NO: 1. Such polypeptides can be designed/selected to exhibit a specific activity, for example an ability to phosphorylate and trap a reporter probe Embodiments of the invention include vectors comprising these nucleic acid molecules. Typically the human thymidine kinase 2 polynucleotide sequence is operably linked to control sequences (e.g. a promoter, an enhancer or the like) that is recognized by a host cell transfected with the vector. Embodiments of the invention also include a host cell transfected with the vector.

Yet another embodiment of the invention is a system for imaging a mammalian cell using positron emission tomography (PET) or single photon emission computed tomography (SPECT), the system comprising a PET reporter gene and a PET reporter probe. In this embodiment, the PET reporter gene encodes a human thymidine kinase such a human thymidine kinase 2 polypeptide (Uniprot ID O00142) and the PET reporter probe comprises a non-naturally occurring analog of thymidine. In this system, the polypeptide encoded by the PET reporter gene is selected for an ability to phosphorylate the non-naturally occurring analog of thymidine. In certain embodiments of this system, the PET reporter gene encodes a thymidine kinase 2 polypeptide comprising amino acid residues 51-265 of SEQ ID NO: 1, wherein the thymidine kinase polypeptide comprises a deletion mutation or a substitution mutation that confers a decreased susceptibility to thymidine triphosphate mediated feedback inhibition as compared to wild type polypeptide shown in SEQ ID NO: 1. In typical embodiment of the invention, the PET reporter gene encodes a thymidine kinase polypeptide comprising amino acid residues 51-265 of SEQ ID NO: 1, and further comprises at least one insertion, substitution or deletion mutation in SEQ ID NO: 1. Optionally, for example, the thymidine kinase polypeptide comprises an amino acid substitution at amino acid residue position 93 and/or amino acid residue position 109 of SEQ ID NO: 1 (e.g. N93D/L109M or N93D/L109F).

Optionally in the system embodiments of the invention, the PET reporter probe is selected from the group consisting of: L-[18^{F}]FMAU, L-[18^{F}]FEAU, L-[18^{F}]FBrVAU, L-[18^{F}]FBU, D-[18^{F}]FMAU, D-[18^{F}]FEAU, D-[18^{F}]FBrVAU, D-[18^{F}]FCU, [18^{F}]FHBG, FFU and FddUrd. In certain embodiments of the invention, the PET reporter probe and/or the PET reporter gene is combined with a pharmaceutically acceptable carrier. In some embodiments, the system is disposed in a kit, the kit comprising a first container comprising a vector that comprises the PET reporter gene, wherein the PET reporter gene is covalently coupled to vector control sequences recognized by a host cell transformed with the vector; and a second container comprising the PET reporter probe.

Yet another embodiment of the invention is a method of imaging a mammalian cell using positron emission tomography (PET) or single photon emission computed tomography (SPECT). In typical embodiments, the method comprise the steps of introducing a reporter gene into a mammalian cell, the reporter gene encoding a thymidine kinase polypeptide comprising amino acid residues 51-265 of SEQ ID NO: 1, introducing a reporter probe comprising a non-naturally occurring analog of thymidine, wherein the thymidine kinase polypeptide encoded by the reporter gene is able to phosphorylate the non-naturally occurring analog of thymidine, and then detecting the reporter probe using positron emission tomography (PET) or single photon emission computed tomography (SPECT). In certain embodiments of the invention, the thymidine kinase polypeptide consists essentially of amino acid residues 51-265 of SEQ ID NO: 1 and further comprises at least one amino acid substitution at amino acid residue position 93 or amino acid residue position 109 of SEQ ID NO: 1 (e.g. N93D, L109M or L109F). In certain embodiments, the thymidine kinase polypeptide comprises a set of amino acid mutations comprising huΔ₁₋₅₀TK2 and N93D/L109M or N93D/L109F. In typical embodiments, the reporter gene is introduced to the mammalian cell by transfecting the mammalian cell with a vector comprising a nucleic acid molecule encoding the thymidine kinase polypeptide and wherein the vector is operably linked to control sequences recognized by the mammalian cell transfected with the vector. Optionally in such methods, the reporter probe is selected from the group consisting of: L-[18^{F}]FMAU, L-[18^{F}]FEAU, L-[18^{F}]FBrVAU, L-[18^{F}]FBU, D-[18^{F}]FMAU, D-[18^{F}]FEAU, D-[18^{F}]FBrVAU, D-[18^{F}]FCU, [18^{F}]FHBG, FFU and FddUrd.

In other embodiments of the invention, enhanced hTK2 PET/SPECT reporter transgenes are delivered into cells of interest ex vivo, using viral vectors or non-viral techniques. In one exemplary embodiment, the delivery method leads to permanent presence of the enhanced htk2 transgenes within the nucleous of the cells. Then the cells of interest overexpressing the enhanced htk2 reporter genes are transplanted through an appropriate route into a living organism where they can be visualized at any time point after administration with a PET or SPECT probes that can detect the expression of the enhanced htk2 reporter genes. The invention also has applications in gene therapy and general biomedical research in discovering intracellular events and analysis of the mechanisms of actions of therapeutic agents.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 provides a bar graph that illustrates market size projections for gene/cell based therapies (sources: Oncology Market Leaders—Analyses and Outlook, Market Report 2008-2013; CST, Inc. estimate).

FIG. 2 provides a schematic and data that illustrate (A) general schematic of ACGT to treat melanoma and (B) serial [¹⁸F]FDG PET scans of a melanoma patient enrolled in an ACGT clinical trial (March: baseline, before treatment; May and June: 1 and 3 months after treatment, respectively). Green circles: regressing lesions, red circle: non-regressing lesion. The high number of lesions in patients with disseminated disease renders tissue biopsies impractical for determining the tissue PK of therapeutic T cells. In contrast, genetic labeling of adoptively transferred anti-tumor T cells with a PET reporter gene would allow clinicians to perform tissue PK measurements and monitor trafficking and homing of these therapeutic cells to melanoma lesions.

FIG. 3 provides a schematic and data that illustrate conceptual, preclinical, and clinical examples of PRG-PRP imaging. (A) PRGs encode a protein that causes probe accumulation on the surface or within the cytoplasm of cells expressing the PRG. HSV1-tk phosphorylates the PRP (i.e. [¹⁸F]FHBG) causing its entrapment. If a cell does not express HSV1-tk, [¹⁸F]FHBG does not accumulate. D₂R serves as a receptor for the [¹⁸F]FESP PRP. (B) Expression of the D₂R and HSV1-sr39tk PRGs in mice, measured by microPET. Mice were injected with either Ad.CMV.HSV1-sr39tk (AdTKm) or Ad.CMV.D2R (AdD2R) and imaged several days later either with FESP or with FHBG (see, e.g. Yaghoubi, S. S., et al. Gene Ther 8:1072-1080, 2001). (C) MRI and PET over MRI superimposed brain images of cytolytic T cells expressing HSV1-tk, infused into the glioma tumor resection site of a patient (see, e.g. Yaghoubi, S. S., et al. Nat Clin Pract Oncol 6:53-58, 2009).

FIG. 4 provides a table that illustrates the mean standard uptake values of the probes in different tissues for ¹⁸F-FHBG and L-¹⁸F-FMAU. Values are calculated at 2 hours post injection. All values are Standard Uptake Value (SUV)+/−SEM.

FIG. 5 provides a schematic that illustrates the mechanism and consequences of PRG immunogenicity. The PRG is transcribed and translated in genetically engineered therapeutic cells; peptides derived from the PRG are displayed on the cell surface in the context of MHC class I molecules. If these peptides have never been encountered by the host immune system (i.e., they are “foreign”), they are detected by CD8+ T cells, which then kill the therapeutic cells, leading to treatment failure. In contrast, if the PRG is sufficiently similar or identical to a gene normally expressed by the host (“self” rather than “foreign”) it is less likely that the therapeutic cells will be detected as “foreign” and eliminated.

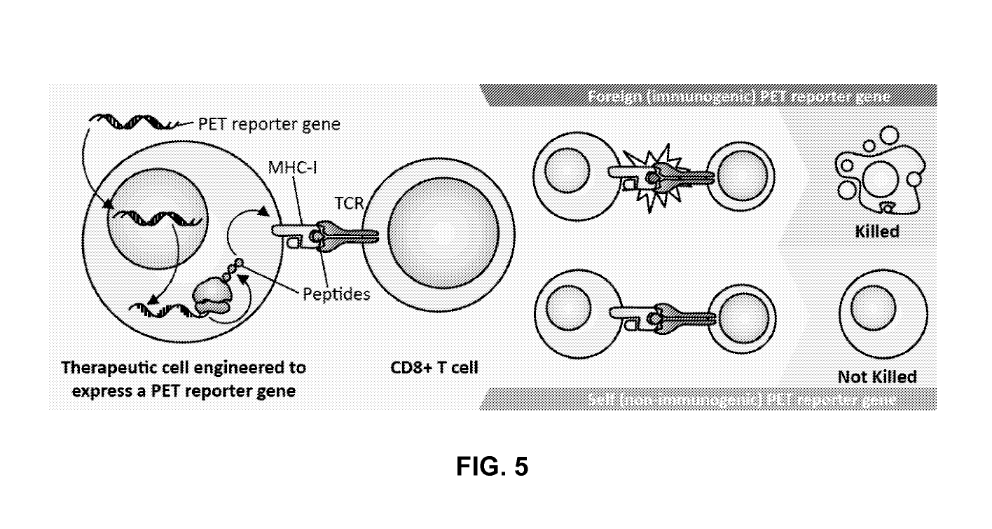

FIG. 6 provides a graph that illustrates data regarding an assay to determine whether candidate probes compete with thymidine for TK1.

FIG. 7 provides a schematic and data that illustrate (A) chemical structures of probe embodiments and (B) biodistribution in immune competent C57/BL6 mice of candidate PET reporter probes. B: bladder; GB: gallbladder; GI: gastro-intestinal tract; K: Kidney; L: Liver; H: Heart.

FIG. 8 provides a schematic and data that illustrate the development of hTK2 N93D as a new PET reporter gene. (A) Rationale for making the N93D point mutation in TK2. Dividing cells have large thymidine triphosphate (dTTP) pools. dTTP inhibits TK2 by a feedback mechanism. The Asparagine (N) residue at position 44 plays a role in the dTTP-negative feedback. Mutating this residue to Alanine (A) reduces the dTTP negative feedback and thus enhances the ability of the PRG to phosphorylate and trap the reporter probe (RP). (B) The TK2-N93D (▪) mutant is more resistant to the dTTP feedback inhibition than WT TK2 (▴). (C) L1210 cell lines transduced with TK2-N93D show increased uptake of L-[¹⁸F]FMAU compared to cells transduced with WT TK2.

FIG. 9 provides graphs of data that illustrate how the overexpression of sr39tk affects cell growth and intracellular dNTP pools. L1210 cells were engineered to express: TK2-N93D (▪), TK2 (▴), HSV1-sr39tk (▾), and control vector eYFP (♦). Various graphs show the growth of the L1210 cells in (A) regular media and (B) regular media supplemented with 10 μM thymidine (dT). Other graphs show total cellular dNTP pools (measured by LC-MS) in cells grown in (C) regular media and (D) regular media supplemented with 10 μM dT; (*=below limit of detection).

FIG. 10 provides images and graphed data that shows in vivo comparisons of PRGs, showing microPET/CT scans (A, C) using L-[¹⁸F]FMAU (upper panels) and [¹⁸F]FHBG (lower panels) and data quantification (B, D). Images were obtained 3 hrs post-injection. B: bladder; GB: gallbladder; GI: gastro-intestinal tract.

FIG. 11 provides images and graphed data that shows a comparison of L-[¹⁸F]FMAU and [¹⁸F]FHBG PET reporter systems in a murine tumor model. (A) L-[¹⁸F]-FMAU microPET/CT scans of L1210-PRG tumors. (B) [¹⁸F]-FHBG microPET/CT scans of L1210-sr39tk tumors. (C) Quantification of tumor uptake in (A) and (B).

FIG. 12 provides images of exemplary human L-[¹⁸F]FMAU PET studies and their comparison with FHBG.

FIG. 13 provides schematics of chemical structures of L-[¹⁸F]-FMAU and [¹⁸F]FHBG.

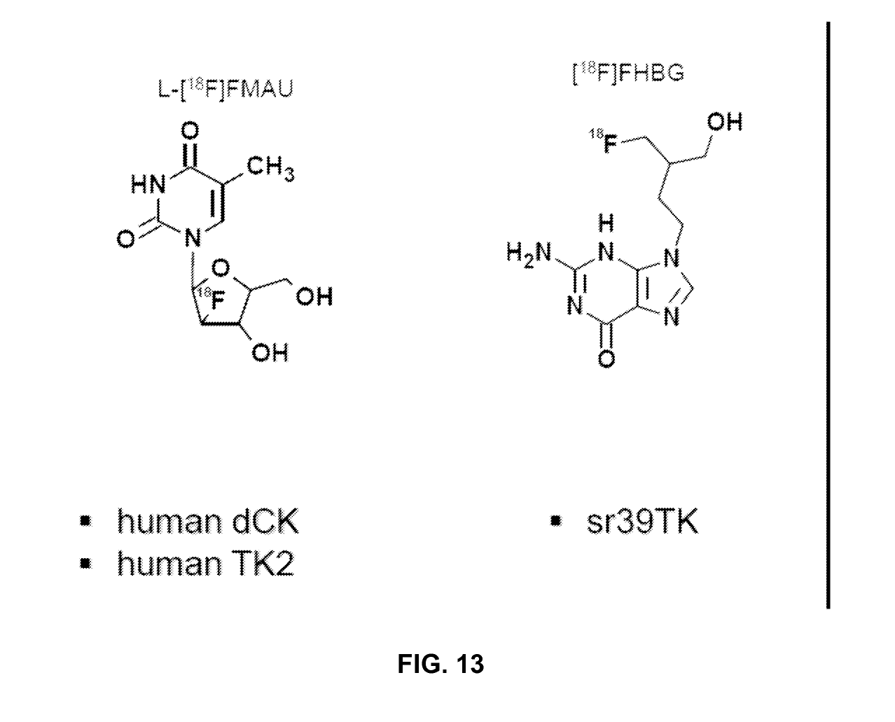

FIG. 14 images that illustrate an evaluation of L-[¹⁸F]-FMAU in humans. Conclusions include: 1) L-FMAU has very low background in abdominal cavity; 2) Liver and Heart uptake limits use in/near these organs; 3) Ethyl- (L-FEAU) and propyl- (L-FPAU) analogs may reduce background in heart and possibly liver.

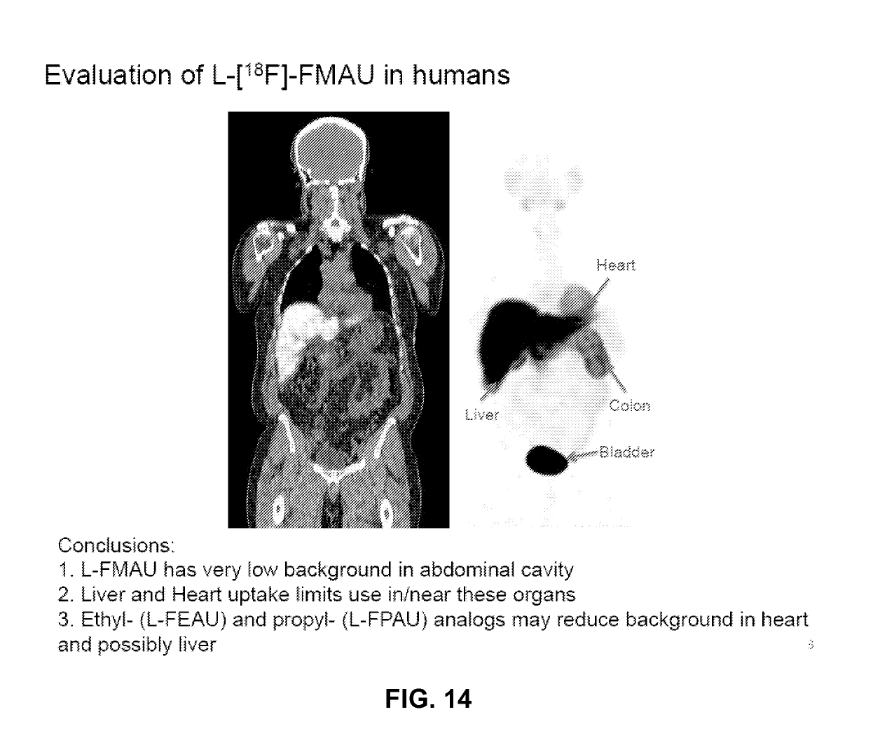

FIG. 15 provides a schematic that illustrates factors to consider when developing nucleoside kinase-based PET reporter genes. Factors affecting sensitivity include: 1) Competition between the probe and the natural substrate (dT); this can be at the level of transport and at the level of the kinase reporter; 2) Negative feedback from downstream product (dTTP). Factors affecting safety include the possibility of inducing imbalances in dNTP pools.

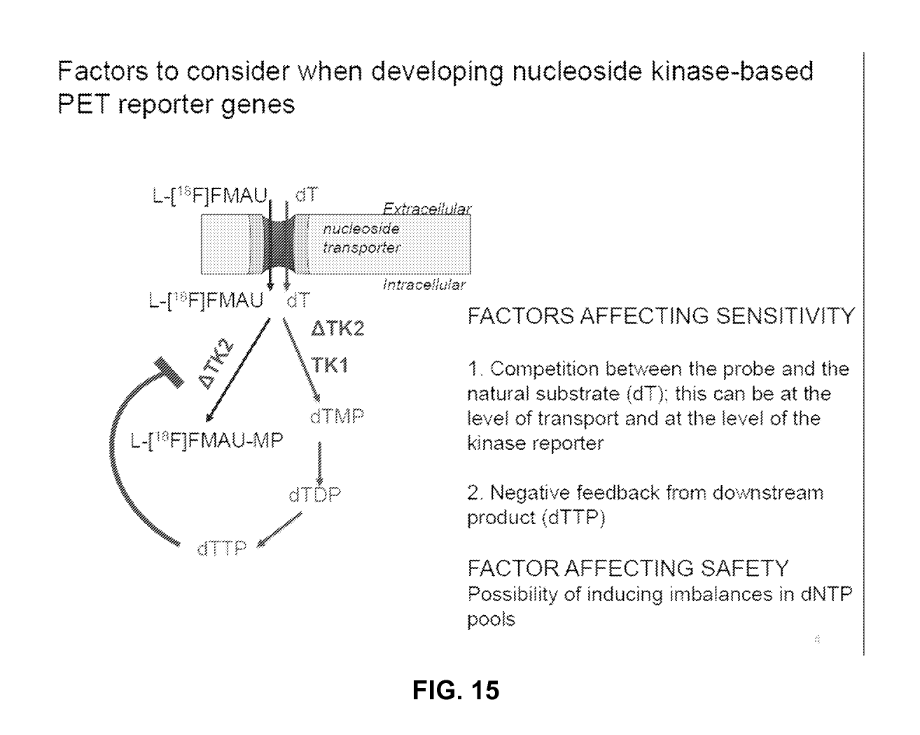

FIG. 16 provides images and graphed data illustrates overcoming the negative feedback of dTTP on ΔTK2 in vivo. L1210-cells were engineered to overexpress the reporter genes. The cells were then injected into the mice in the areas shown. The tumors that developed were then imaged with the L-FMAU 7 days post injection. The scans were done three hours post injection of the probe.

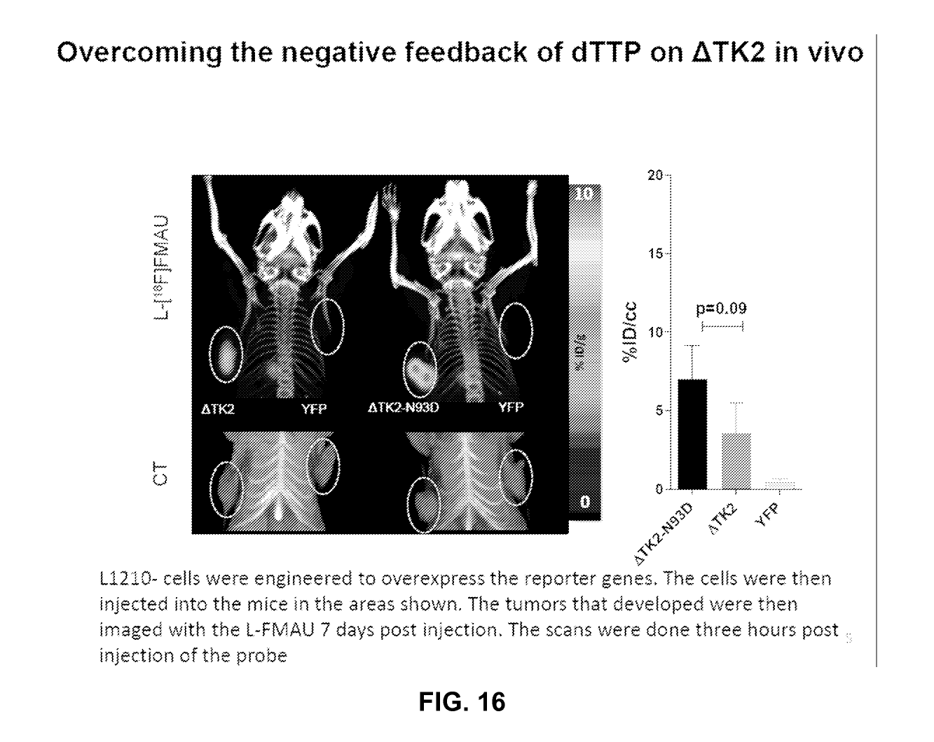

FIG. 17 provides graphed data illustrates TK2 mutations that improve the selectivity of ΔTK2 for L- versus D-nucleosides. This is in an uptake assay using L1210 cells engineered to overexpress the listed reporter genes. 250,000 cells were incubated for one hour with ¹⁸F-L-FMAU in the presence or absence of 10 μM dT. The cells were then washed and the amount of ¹⁸F-LFMAU taken up by the cells obtained by quantifying the uptake with a gamma counter.

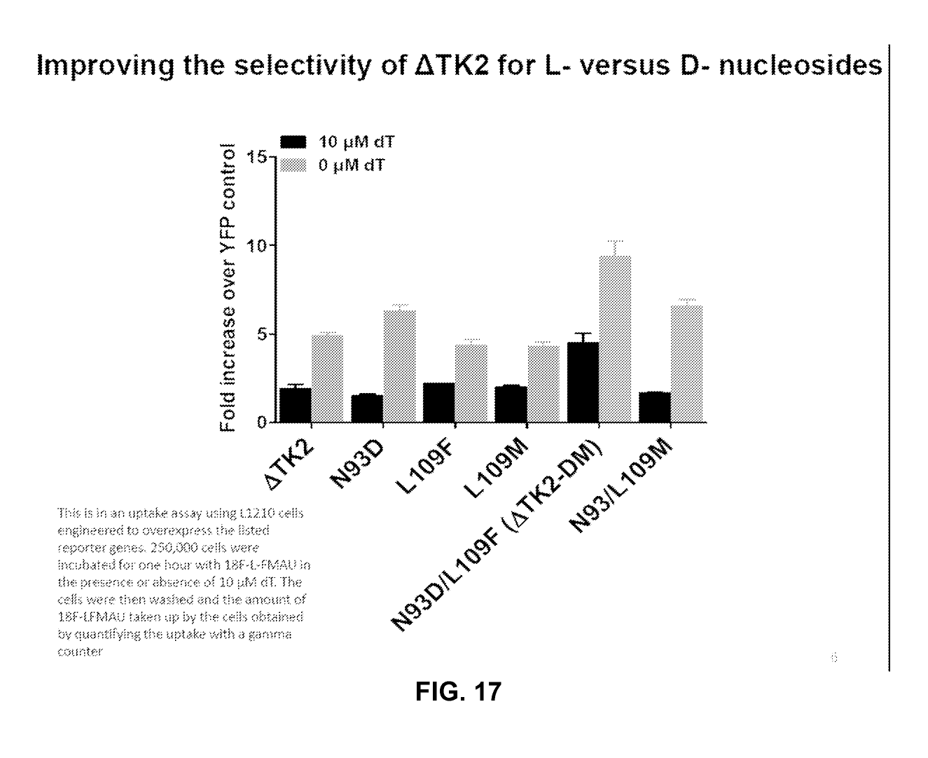

FIG. 18 provides images and graphed data illustrates an evaluation of ΔTK2-DM in vivo. Similar to FIG. 16, but with the genes and probes shown.

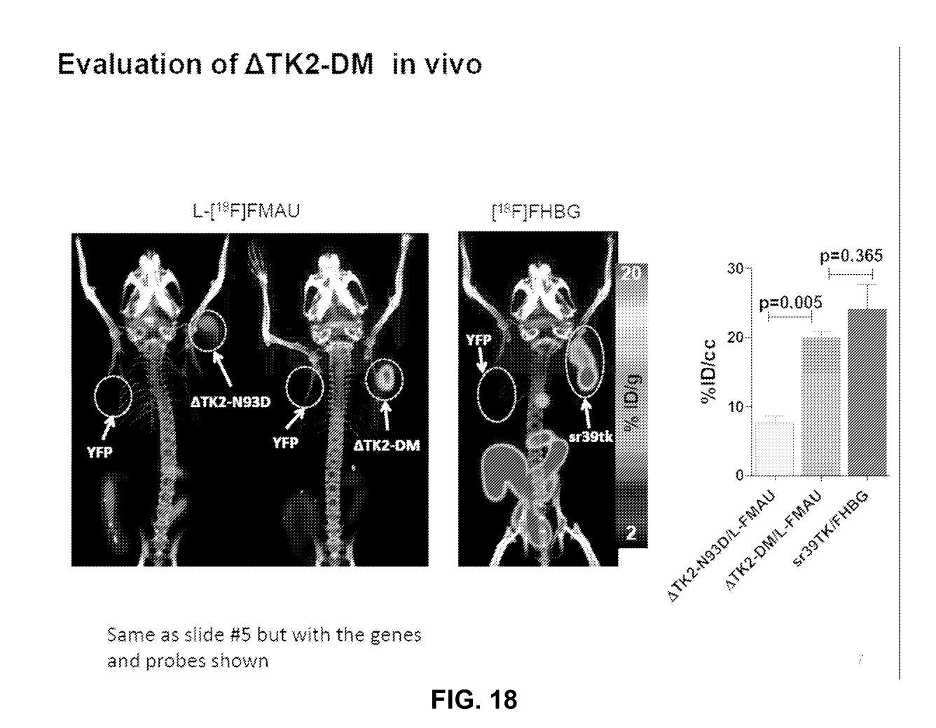

FIG. 19 provides graphed data that illustrates that, in contrast to sr39tk, the double mutant TK2 does not alter nucleotide pools. L1210 cells transduced to overexpress the listed genes were grown in RPMI media (+/−10 μM dT) for 48 hours. The cells were then harvested and pyrimidine nucleotide pools were obtained via a DNA polymerase assay.

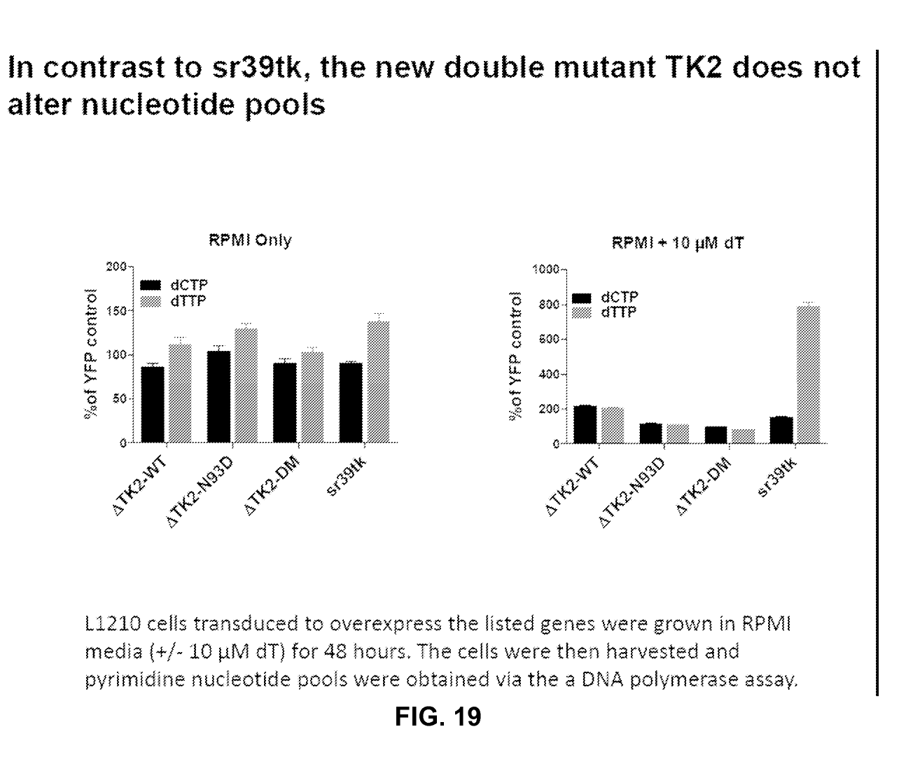

FIG. 20 provides images that illustrate a composite of healthy human volunteer images for the PET tracer [¹⁸F]L-FMAU. This was a first in human study. On the top is shown the minutes after injection of the PET tracer that the images were acquired. For example, the first PET/CT scan was acquired 25-55 minutes after [¹⁸F]L-FMAU injection. The images show uptake of [¹⁸F]L-FMAU, mainly in its clearance routes, liver and bladder, with relatively low accumulation in intestines and some accumulation in the heart.

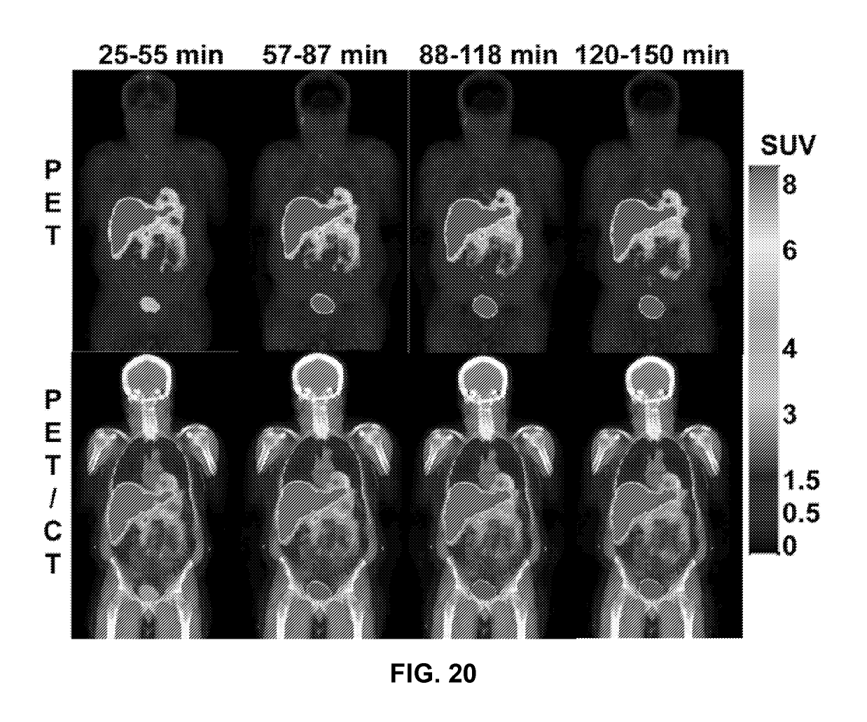

FIG. 21 provides illustrative human TK2 amino acid sequences. The wild type TK2 amino acid and polynucleotide sequences are shown in SEQ ID NO: 1 and SEQ ID NO: 2 respectively. In SEQ ID NO: 1, amino acid residues 51-265 are shown in bold and residues 93 and 109 are underlined. In SEQ ID NO: 2, the codons that encode amino acid residues 51-265 of huTK2 are shown in bold. Sequences of illustrative TK2 mutants are also shown, namely huΔTK2-N93D (SEQ ID NO: 3) and TK2-N93D/L109F (SEQ ID NO: 4).

FIG. 22 provides a Table that illustrates a table of existing PET reporter systems.

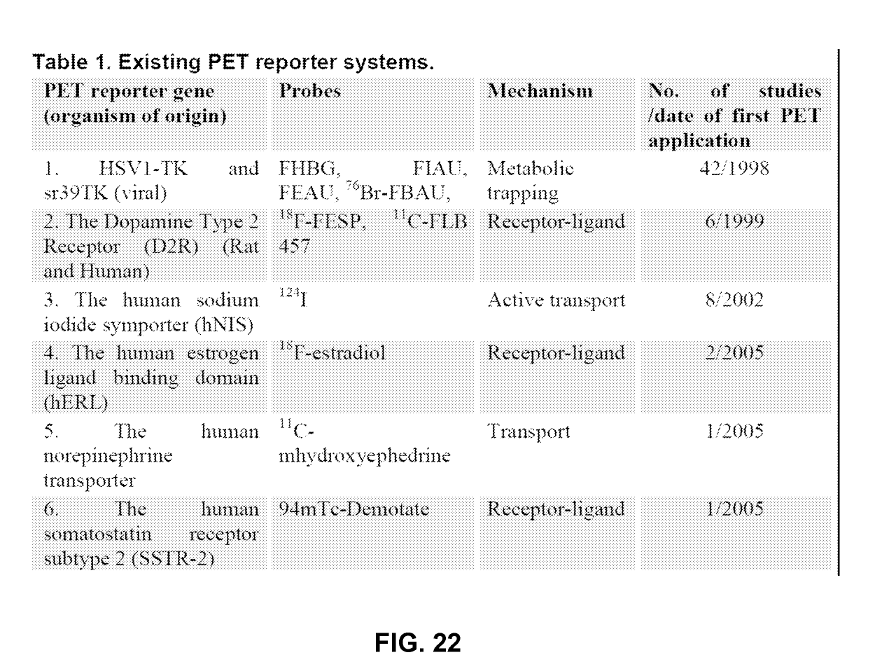

FIG. 23 provides a schematic and graphs that illustrate the development of hTK2 N93D as a new PET reporter gene. (A) Rationale for making the N93D point mutation in TK2. (B) The TK2-N93D mutant is more resistant to the dTTP feedback inhibition than wild type (WT) TK2. (C) [¹⁸F]-L-FMAU uptake assay using L1210 cell lines transduced with TK2-N93D (▪), TK2 (▴), sr39TK (▾), and control vector (♦).

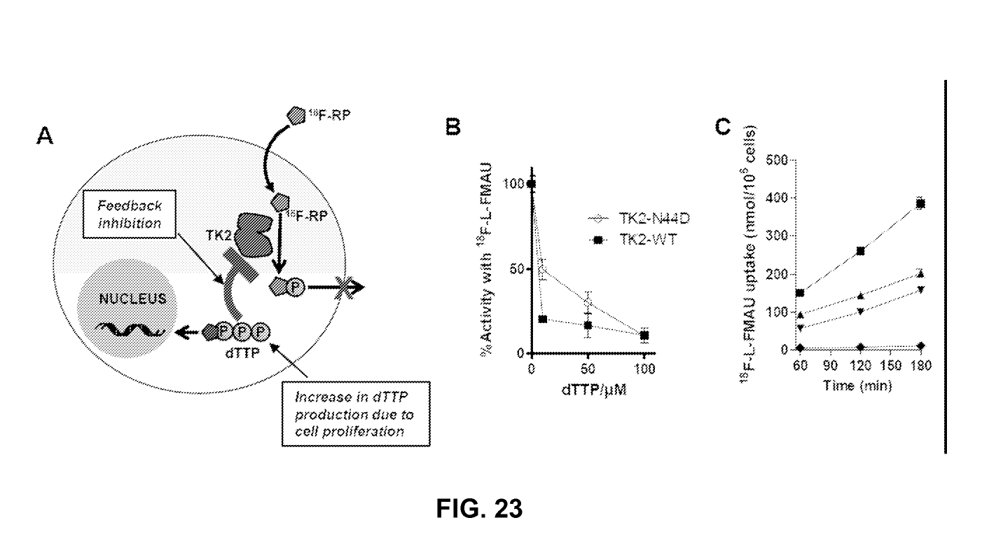

FIG. 24 provides images and graphs that show in vivo comparison of sr39tk and TK2 N93D PRGs. MicroPET/CT scans (A, C) using [¹⁸F]-L-FMAU (upper panels) and [¹⁸F]FHBG (lower panels); data quantification (B, D). Images were obtained 3 hrs post-injection. B: bladder; GB: gallbladder; GI: gastro-intestinal tract.

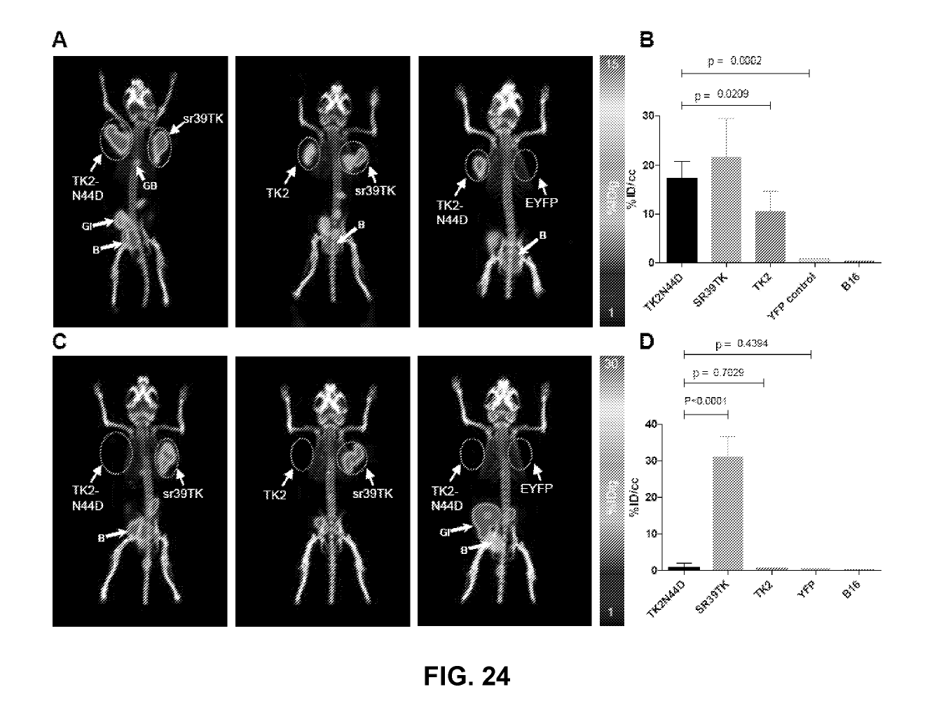

FIG. 25 provides schematics and images that illustrate (A) chemical structures for four embodiments of PET probes. (B) biodistribution of PRPs in mice.

FIG. 26 provides schematics and images that illustrate the biodistribution of L-¹⁸F-FMAU and ¹⁸F-FHBG in mice. (A) chemical structures of L-18F-FMAU and 18F-FHBG are shown. (B) MicroPET/CT scans of C57/BL6 mice 3 h after injection of L-¹⁸F-FMAU (left) and ¹⁸F-FHBG (right) are shown. Images are co-registered displays of the separate microPET and microCT scans. Quantifications of the PET signals are listed in FIG. 33. B: bladder; GB: gallbladder. % ID/g: % injected dose/g.

FIG. 27 provides schematics and images that illustrate the evaluation of a TK2-N93D mutant. (A) model of WT TK2 bound with L-dT in both the closed (green) and open (pink) conformation of the enzyme. ADP is bound in the phosphate donor pocket shown in this model. The enzyme is active in the closed conformation, which is stabilized by bonds between residues Asn-93 and Glu-200. When asparagine 93 is mutated to a glutamine, the bonds are disrupted, and the enzyme is predicted to switch to an open (inactive) conformation. (B) L-FMAU kinase assay using recombinant WTTK2 and TK2-N93D in the presence of increasing concentrations of dTTP is shown. (C) L-18F-FMAU uptake assay using WT TK2- or TK2-N93D-expressing L1210 cells is shown. Probe uptake values are reported relative to a control L1210 cell line that expresses YFP. Results are for a representative experiment or n=2 experiments. (D) L-¹⁸F-FMAU microPET/CT scans of mice bearing L1210 tumors engineered to express various PRGs (TK2, L1210-TK2; N93D, L1210-TK2-N93D; YFP, L1210-YFP). (E) Quantification of PET scans from panel D. % ID/g: % injected dose/g.

FIG. 28 provides a graph of data that illustrates the kinetic analyses of recombinant TK2 mutants with D-dT, L-dT, and L-FMAU. Values were measured at 37° C. using 1 mM ATP as phosphoryl donor. k_{cat} is in s⁻¹, Kₘ is in μM, and k_{cat}/Kₘ is in M⁻¹×s⁻¹.

FIG. 29 provides a schematic and graphs of data that illustrates the evaluation of L109F and N93D/L109F TK2 mutants. (A) a homology model of TK2 bound with L-dT (pink) and dT (green) is shown. The TK2 model (solid residues) is overlaid on a crystal structure of dCK (light colored residues) with bound substrates. The TK2 residue Leu-109 is highlighted in gold. (B) a L-FMAU kinase assay using recombinant TK2-L109F and TK2-N93D/L109F in the presence of increasing dTTP concentrations is shown. (C) shown is an in vitro L-¹⁸F-FMAU uptake assay using L1210 cells expressing either TK2-N93D, TK2-L109F, or TK2-N93D/L109F. The assay was done in either the presence or absence of 5 μMD-dT. Probe uptake values are reported relative to a control L1210 cell line that expresses YFP. Results are for a representative experiment or n=2 experiments. P=0.005 between N93D and N93D/L109F in the presence of 0 μM dT, and p=0.0008 between N93D and N93D/L109F in the presence of 5 μM dT.

FIG. 30 provides images and graphs that illustrate a comparison of ΔTK2/L-¹⁸F-FMAU and sr39tk/¹⁸F-FHBG PET reporter gene systems. (A) L-¹⁸F-FMAU microPET/CT scans of mice bearing L1210 tumors engineered to express various TK2-based PRGs. (B) ¹⁸F-FHBG microPET/CT scans of mice bearing L1210 tumors engineered to express sr39tk. (C) Quantification of probe uptake in L1210 tumors from (A) and (B). % ID/g: % injected dose/g.

FIG. 31 provides images that illustrate the biodistribution of L-¹⁸F-FMAU and ¹⁸F-FHBG in humans. PET/CT scans of a healthy female (left) and a healthy male (right) volunteer 2 hours after injection of (A) L-¹⁸F-FMAU and pretreatment glioma patient 2 h after injection of (B) ¹⁸F-FHBG. B: bladder; GB: gallbladder; L: liver; M: myocardium; SUV: standard uptake value.

FIG. 32 provides a schematic that illustrates an embodiment of the synthesis of L-¹⁸F-FMAU.

FIG. 33 provides a table that illustrates the calculated accumulation of PET reporter probes in tissues of C57/BL6 mice 3 hours post-injection. All values are % ID/cc+/−SEM.

FIG. 34 provides a table that illustrates the kinetics of the phosphorylation of D and L-nucleosides by the TK2 mutants. Kinase assays were performed with recombinant protein with 200 μM of the listed substrate.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

As described herein, “gene therapy” involves all forms of administering a therapeutic transgene, for example, viral vectors, naked DNA, liposomes, nanoparticles, and cells. The term “TG” (therapeutic genes) is used to refer to all forms of transgenes that are delivered to achieve a therapeutic outcome. The term “TC” (therapeutic cells) is used to indicate cells that are delivered to achieve a therapeutic outcome. TC may be genetically engineered to express a transgene to achieve its desired therapeutic effect or may have its own endogenous therapeutic mechanism.

Gene expression is defined herein as transcription of a gene into its messenger RNA (mRNA) and/or translation of its mRNA into protein. Monitoring the kinetics of a gene is defined herein as detecting the presence and locations of its expression and/or the magnitude of its expression in living mammals. Monitoring the kinetics of a cell is defined herein as detecting its presence or location, determining its survival, measuring its proliferation, and/or tracking changes in its characteristics over time in a living mammal.

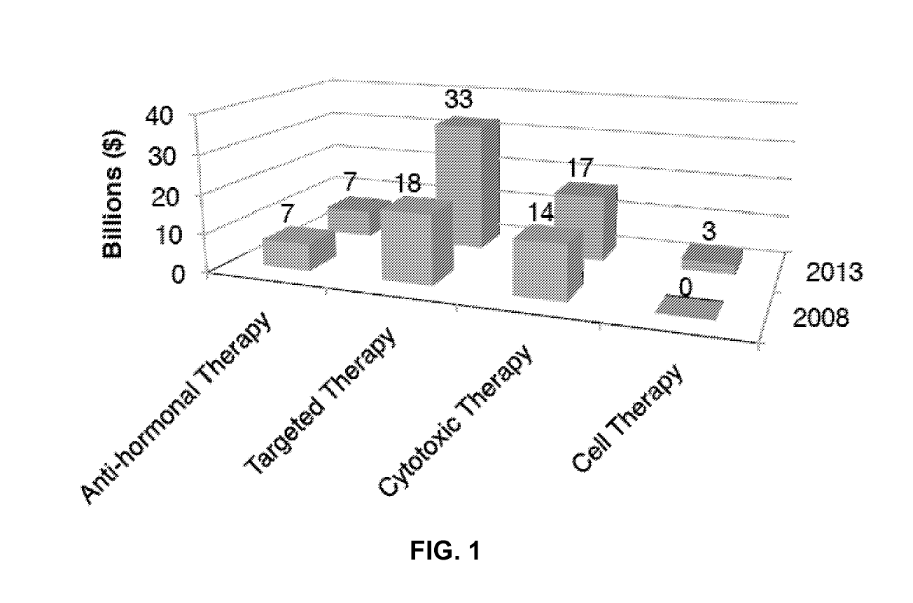

Gene and cell-based therapies may overcome the limitations of conventional treatments for many types of diseases including but not limited to cancer and autoimmune, cardiovascular and neurological disorders. Therefore their market share is projected to grow significantly in the near future (FIG. 1). For either “gene therapy”, or for “cell therapy”, one would also want an imaging technology to localize and quantify the therapeutic product throughout the body.

Positron emission tomography (PET) reporter gene imaging can be used to non-invasively monitor cell-based therapies. Therapeutic cells engineered to express a PET reporter gene (PRG) specifically accumulate a PET reporter probe (PRP) and can be detected by PET imaging. Expanding the utility of this technology requires the development of new non-immunogenic PRGs. In one embodiment of the present invention, a PRG-PRP system is provided that employs, as the PRG, a mutated form of human thymidine kinase 2 (TK2) and 2′-deoxy-2′-¹⁸F-5-methyl-1-O-L-arabinofuranosyluracil (L-¹⁸F-FMAU) as the PRP. In one embodiment, a TK2 double mutant (TK2-N93D/L109F) is provided that efficiently phosphorylates L-¹⁸F-FMAU. The N93D/L109F TK2 mutant has lower activity for the endogenous nucleosides thymidine and deoxycytidine than wild type TK2, and its ectopic expression in therapeutic cells is not expected to alter nucleotide metabolism. Imaging studies in mice indicate that the sensitivity of the new human TK2-N93D/L109F PRG is comparable with that of a widely used PRG based on the herpes simplex virus 1 thymidine kinase.

Development of PET reporter gene systems is a highly dynamic and innovative field in molecular imaging. The development of the hTK2-N93D PRG provided herein, started with an innovative concept wherein development of a new catalytic PET reporter system should not start with the PRG component, but rather with the identification of candidate PRPs. Only after the identification of optimal PRPs, should PRGs be engineered to provide maximal sensitivity and specificity. Specifically, PRPs should satisfy two criteria: (i) the probe should be amenable to routine ¹⁸F labeling, and (ii) the probe should demonstrate a high specific signal-to-background ratio. Furthermore, following injection, the candidate PRP should have access to the tissues but should also be rapidly cleared if the PRG is not expressed (i.e., the PRP should accumulate rapidly and specifically only in cells and tissues genetically engineered to express the PRG).

Embodiments of the present invention include a PET/SPECT reporter gene system that uses an enhanced version of human mitochondrial thymidine kinase 2 (htk2) expressed in cytoplasm to preferentially trap PET/SPECT radiolabeled L and D-enantiomer analogs of the natural substrate thymidine. In addition, the enhanced htk2 reporter gene can serve as a safety gene to destroy cells expressing it when they malfunction. Endogenous hTK2 enzyme provides the first phosphorylation step in the salvage pathway of deoxyribonucleosides. A method is also provided for mutating the htk2 gene to enhance the catalytic activity of its enzyme product for phosphorylation of several thymidine analogs.

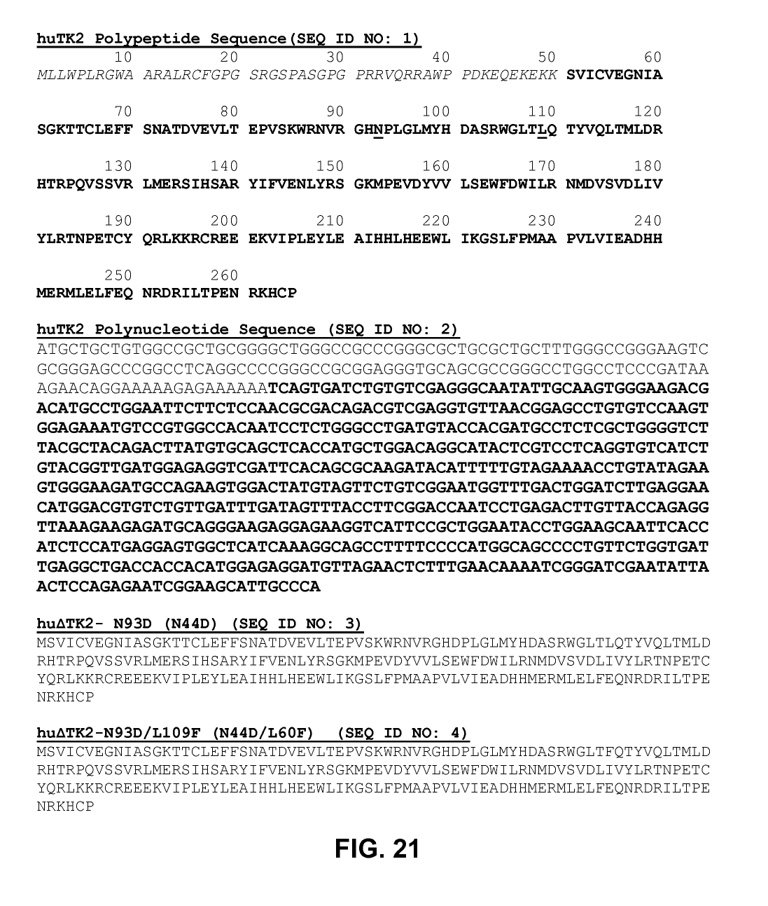

The invention disclosed herein has a number of embodiments. One embodiment is a human thymidine kinase 2 polypeptide comprising amino acid residues 51-265 of SEQ ID NO: 1, wherein the thymidine kinase polypeptide comprises an amino acid substitution at amino acid residue position 93 and/or amino acid residue position 109 of SEQ ID NO: 1. In typical embodiments, the thymidine kinase polypeptide is selected for an ability to exhibit an activity as disclosed herein, for example an ability to phosphorylate and/or trap a non naturally occurring analog of thymidine such as 2′-deoxy-2′-18^{F}-5-methyl-1-β-L-arabinofuranosyluracil. In certain embodiments of the invention, the thymidine kinase polypeptide does not include the first 50 amino acids of SEQ ID NO: 1 and comprises an amino acid substitution at amino acid residue position 93 of SEQ ID NO: 1 (e.g. N93D) and an amino acid substitution at amino acid residue position 109 of SEQ ID NO: 1 (e.g. L109M or L109F). Optionally, the polypeptide comprises a set of amino acid mutations such as a polypeptide huΔ₁₋₅₀TK2-N93D/L109M (see, e.g. FIG. 21).

Other embodiments of the invention include a nucleic acid molecule comprising DNA encoding a human thymidine kinase 2 polypeptide comprising amino acid residues 51-265 of SEQ ID NO: 1. Typically in these embodiments, the thymidine kinase polypeptide comprises a deletion such as huΔ₁₋₅₀TK2, an insertion such as the N-terminal methionine in SEQ ID NO: 3 and 4, an amino acid substitution at amino acid residue position 93 and/or amino acid residue position 109 of SEQ ID NO: 1 etc. In addition to these structural features, such polypeptides can also be selected to exhibit a specific activity, for example an ability to trap or phosphorylate a non naturally occurring analog of thymidine such as L-[18^{F}]FMAU, L-[18^{F}]FEAU, L-[18^{F}]FBrVAU, L-[18^{F}]FBU, D-[18^{F}]FMAU, D-[18^{F}]FEAU, D-[18^{F}]FBrVAU, D-[18^{F}]FCU, [18^{F}]FHBG, FFU and FddUrd.

Embodiments of the invention include vectors comprising these nucleic acid molecules. A wide variety of vectors can be adapted for use with embodiments of the present invention. Illustrative viral vectors include, for example, adenovirus-based vectors (see, e.g. Cantwell (1996) Blood 88:4676 4683; and Ohashi (1997) Proc Natl Acad Sci USA 94:1287 1292), Epstein-Barr virus-based vectors (see, e.g. Mazda (1997) J Immunol Methods 204:143 151), adenovirus-associated virus vectors, Sindbis virus vectors (see, e.g. Strong (1997) Gene Ther 4:624 627), herpes simplex virus vectors (see, e.g. Kennedy (1997) Brain 120:1245 1259) and retroviral vectors (see, e.g. Schubert (1997) Curr Eye Res 16:656 662). Typically the human thymidine kinase 2 polynucleotide sequence is operably linked to control sequences in the vector (e.g. a promoter, an enhancer or the like) that is recognized by a host cell transfected with the vector. Optionally, for example, the human thymidine kinase 2 polynucleotide sequence is operably linked to tissue specific control sequences in a vector so that it is selectively expressed in cells of a specific tissue lineage. Embodiments of the invention also include a host cell transfected with the vector.

Embodiments of the invention also include compositions of matter that comprise a non naturally occurring analog of thymidine, for example an imaging compound disclosed herein. Illustrative compositions can comprise L-[18^{F}]FMAU, L-[18^{F}]FEAU, L-[18^{F}]FBrVAU, L-[18^{F}]FBU, D-[18^{F}]FMAU, D-[18^{F}]FEAU, D-[18^{F}]FBrVAU, D-[18^{F}]FCU, [18^{F}]FHBG, FFU or FddUrd. In certain embodiments of the invention, the PET reporter probe is combined with a pharmaceutically acceptable carrier.

Yet another embodiment of the invention is a system for imaging a mammalian cell using positron emission tomography (PET) or single photon emission computed tomography (SPECT), the system comprising a PET reporter gene and a PET reporter probe. In this embodiment, the PET reporter gene encodes a human thymidine kinase such a human thymidine kinase 2 polypeptide (Uniprot ID O00142) and the PET reporter probe comprises a non-naturally occurring analog of thymidine. In such systems, the polypeptide encoded by the PET reporter gene is selected for an ability to phosphorylate a non-naturally occurring analog of thymidine. In certain embodiments of this system, the PET reporter gene encodes a thymidine kinase 2 polypeptide comprising amino acid residues 51-265 of SEQ ID NO: 1, and further comprises a deletion mutation or a substitution mutation that confers a decreased susceptibility to thymidine triphosphate mediated feedback inhibition as compared to wild type SEQ ID NO: 1. In typical embodiment of the invention, the PET reporter gene encodes a thymidine kinase polypeptide comprising amino acid residues 51-265 of SEQ ID NO: 1, and further comprises at least one insertion, substitution or deletion mutation in SEQ ID NO: 1. Optionally, for example, the thymidine kinase polypeptide comprises an amino acid substitution at amino acid residue position 93 and/or amino acid residue position 109 of SEQ ID NO: 1 (e.g. N93D/L109M or N93D/L 109F).

Optionally in the system embodiments of the invention, the PET reporter probe is selected from the group consisting of: L-[18^{F}]FMAU, L-[18^{F}]FEAU, L-[18^{F}]FBrVAU, L-[18^{F}]FBU, D-[18^{F}]FMAU, D-[18^{F}]FEAU, D-[18^{F}]FBrVAU, D-[18^{F}]FCU, [18^{F}]FHBG, FFU and FddUrd. In certain embodiments of the invention, the PET reporter probe and/or the PET reporter gene is combined with a pharmaceutically acceptable carrier. The PET reporter probe and/or the PET reporter gene may be administered as a pharmaceutical composition in a variety of forms including, but not limited to, liquids, powders, suspensions, tablets, pills, capsules, sprays and aerosols. The pharmaceutical compositions may include various pharmaceutically acceptable additives including, but not limited to, carriers, excipients, binders, stabilizers, antimicrobial agents, antioxidants, diluents and/or supports. Examples of suitable excipients and carriers are described, for example, in “Remington's Pharmaceutical Sciences,” Mack Pub. Co.; 17th edition New Jersey (2011). Some suitable pharmaceutical carriers will be evident to a skilled worker and include, e.g., water (including sterile and/or deionized water), suitable buffers (such as PBS), physiological saline or the like. A pharmaceutical composition or kit of the invention can contain other pharmaceuticals, in addition to the compositions of the invention.

Another embodiment of the invention is a kit useful for any of the methods disclosed herein, either in vitro or in vivo. Such a kit can comprise one or more of the compositions of the invention. Optionally, the kits comprise instructions for performing the method. Optional elements of a kit of the invention include suitable buffers, pharmaceutically acceptable carriers, or the like, containers, or packaging materials. The reagents of the kit may be in containers in which the reagents are stable, e.g., in lyophilized form or stabilized liquids. The reagents may also be in single use form, e.g., in single dosage form. In some embodiments, a system as disclosed herein is disposed in a kit, the kit comprising a first container comprising a vector that comprises the PET reporter gene, wherein the PET reporter gene is covalently coupled to vector control sequences recognized by a host cell transformed with the vector; and a second container comprising the PET reporter probe.

Yet another embodiment of the invention is a method of imaging a mammalian cell using positron emission tomography (PET) or single photon emission computed tomography (SPECT). In typical embodiments, the method comprise the steps of introducing a reporter gene into a mammalian cell, the reporter gene encoding a thymidine kinase polypeptide comprising amino acid residues 51-265 of SEQ ID NO: 1, introducing a reporter probe comprising a non-naturally occurring analog of thymidine, wherein the thymidine kinase polypeptide encoded by the reporter gene is able to phosphorylate the non-naturally occurring analog of thymidine, and then detecting the reporter probe using positron emission tomography (PET) or single photon emission computed tomography (SPECT). In certain embodiments of the invention, the thymidine kinase polypeptide consists essentially of amino acid residues 51-265 of SEQ ID NO: 1 and further comprises at least one amino acid substitution at amino acid residue position 93 or amino acid residue position 109 of SEQ ID NO: 1 (e.g. N93D, L109M or L109F). Optionally the huTK polypeptide is fused to a heterologous amino acid sequence. In certain embodiments, the thymidine kinase polypeptide comprises a set of amino acid mutations comprising huΔ₁₋₅₀TK2 and N93D/L109M or N93D/L109F (see, e.g. the embodiments of this polypeptide shown in FIG. 21). In typical embodiments, the reporter gene is introduced to the mammalian cell by transfecting the mammalian cell with a vector comprising a nucleic acid molecule encoding the thymidine kinase polypeptide and wherein the vector is operably linked to control sequences recognized by the mammalian cell transfected with the vector. Optionally in such methods, the reporter probe is selected from the group consisting of: L-[18^{F}]FMAU, L-[18^{F}]FEAU, L-[18^{F}]FBrVAU, L-[18^{F}]FBU, D-[18^{F}]FMAU, D-[18^{F}]FEAU, D-[18^{F}]FBrVAU, D-[18^{F}]FCU, [18^{F}]FHBG, FFU and FddUrd.

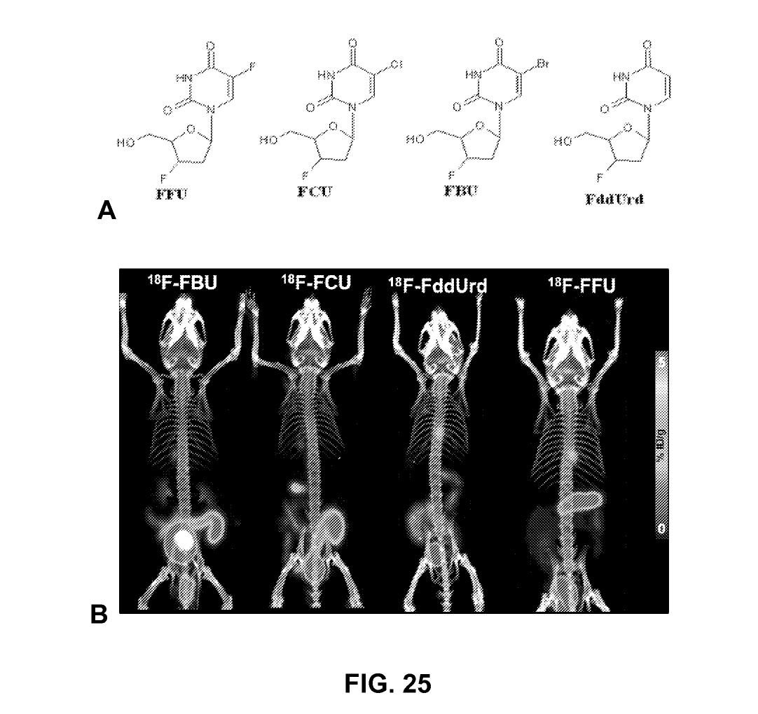

In certain embodiments of the present invention, four novel PET probes are also provided: ¹⁸F-FBU (3′-[¹⁸F]fluoro-2′,3′-dideoxy-5-bromouridine), ¹⁸F-FCU (3′-[¹⁸F]fluoro-2′,3′-dideoxy-5-chlorouridine), ¹⁸F-FddUrd (3′-[¹⁸F]fluoro-2′,3′-dideoxy-uridine), and ¹⁸F-FFU (3′-[¹⁸F]fluoro-2′,3′-dideoxy-5-fluorouridine). Their chemical structures are shown in FIG. 25A. The four fluorine-18 radiolabeled PRPs cannot be phosphorylated by endogenous mammalian thymidine kinases. Therefore, these PRPs will not accumulate in human cells, thus fulfilling a critical requirement for cell tracking applications using PET. FIG. 25B shows that these PRPs have excellent biodistribution in mice, as indicated by the lack of probe retention in all normal tissues, except for the clearance routes such as the GI tract and the bladder. To determine radiation dosimetry, which is required to obtain approval to conduct pharmacokinetic studies in healthy volunteers, exemplary dynamic PET scans in mice have been performed for each of the four PRPs.

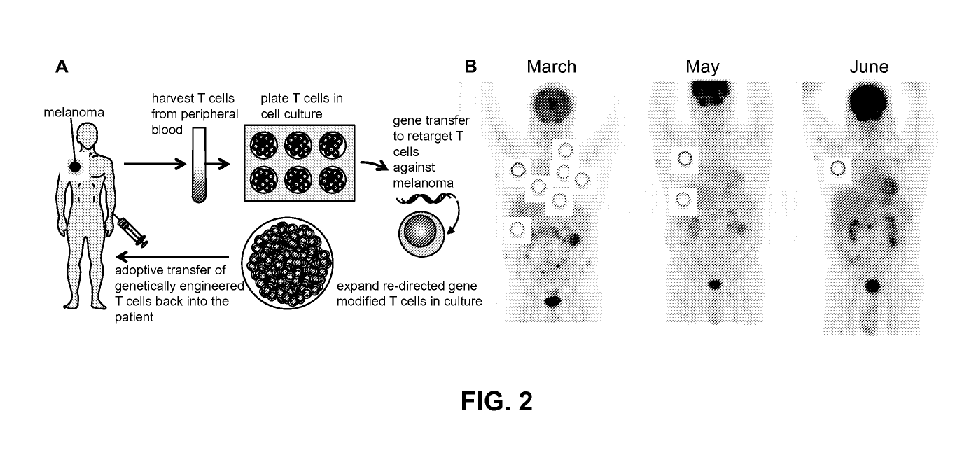

In some embodiments, the enhanced hTK2 PET/SPECT reporter transgenes are delivered into cells of interest ex vivo, using viral vectors or non-viral techniques. In an exemplary embodiment, the delivery method leads to permanent presence of the enhanced htk2 transgenes within the nucleous of the cells. Then the cells of interest overexpressing the enhanced htk2 reporter genes are transplanted through an appropriate route into a living organism where they can be visualized at any time point after administration with a PET or SPECT probes that can detect the expression of the enhanced htk2 reporter genes. FIG. 2 illustrates imaging of implanted xenografts of cells expressing one embodiment of the enhanced htk2 (tk2-N93D) and another commonly used PET reporter gene, HSV1-sr39tk with the PET reporter probes L-[¹⁸F]FMAU and [¹⁸F]FHBG. The advantage of tk2-N93D is that it should have a significantly lower immunogenicity in humans compared to viral TK polypeptides (e.g. as PET reporter genes of viral origin HSV1-sr39tk are typically observed to be immunogenic in humans). The invention also has applications in gene therapy and general biomedical research in discovering intracellular events and analysis of the mechanisms of the actions of therapeutic agents. The PRG can also be delivered directly into specific cells in vivo.

This invention provides several improvements over existing PET/SPECT reporter gene approaches. First, the PET/SPECT reporter genes are based on a human gene and thus are less likely to induce unwanted immune responses if used in human subjects. Second, the invention includes novel L-enantiomers of thymidine analogs that have improved pharmacokinetics, because as unnatural substrates of mammalian TK enzymes they have much less background accumulation; hence superior signal-to-noise ratio in vivo compared to existing probes.

The sensitivity of the described mutant htk2 imaging reporter genes can be evaluated through cell culture assays and PET imaging in mouse tumor models. The invention has been successfully tested in Baf-3 cells with [¹⁸F]L-FEAU and [¹⁸F]L-FMAU. Further tests regarding the effect of expressing these reporter genes on various cells can be used to determine other potential targets. The examples disclosed herein demonstrate the superior pharmacokinetics of the L-enantiomer PET probes provided herein. Several enhanced htk2 mutant reporter genes have been designed and tested. Though the examples herein describe different probes and investigate the L probes mainly in mice, other mammals and in particular, humans are also within the scope of the invention. Silico models can be used to derive additional enhanced mutants from human htk2.

Applications of the present invention include non-invasive, whole body PET/SPECT-based tracking of tumor cells and therapeutic cells, including but not limited to stem cells or multiple types of immune cells. The invention can be used to optimize therapeutic strategies in gene or cell therapy or can be used as a tool in biomedical research in immunocompetent animals. The technologies disclosed herein are generalizable to many types of gene and cell-based therapies for cancer, regenerative medicine (neurological, cardiovascular, hematological, endocrine), and infectious diseases such as AIDS. The non-immunogenic PET reporter systems disclosed herein can enable investigators to observe the activity and predict consequences of emerging gene and cell therapies, and can provide essential information to accelerate the development and effective clinical implementation of these therapies.

The new poorly-immunogenic human TK PRGs (as compared to viral TK polypeptides) disclosed herein can serve purposes beyond gene and cell therapy monitoring. In general, these PRGs can be used to image gene expression, therefore, they are also useful in imaging the regulation of endogenous genes, imaging gene expression in transgenic mice, imaging protein-protein interactions, imaging signal transduction, and many other applications that involve imaging gene expression. In addition, their ability to tag cells for kinetics monitoring makes them useful for applications beyond just monitoring pharmacokinetics of therapeutic cells. These PRGs can be used to monitor kinetics of almost any type of cell that has been genetically engineered to express them either in vitro or in vivo. For example, they can also be used to track metastasis of tumor cells or migration and proliferation of human immune cells, for diagnostic or investigational purposes. In another aspect of the present invention, end-user-ready PET Reporter Gene (PRG) delivery kits co-marketed with PET Reporter Probes (PRP) are provided that enable whole body PK and therapeutic outcome information.

Embodiments and aspects of the invention are disclosed in the following Examples.

### EXAMPLES

### Example 1

### Illustrative Methods and Materials

PET Reporter Probe (PRG-PRP) Systems.

Embodiments of the invention include a new PRG comprising at least one point mutant (e.g. N93D) in the human thymidine kinase 2 (tk2) gene. L-[¹⁸F]FMAU and L-[¹⁸F]FEAU, two hTK2-N93D substrates, are the new PRPs for use with this PRG. One can evaluate L-[¹⁸F]FMAU and L-[¹⁸F]FEAU in vitro, in cell culture, and in vivo. One can also analyze the potential immunogenicity of the hTK2-N93D PRG, and, if necessary, one can design hTK2-N93D variants unable to elicit T cell-mediated immunity in humans.

1.1. Preliminary Data.

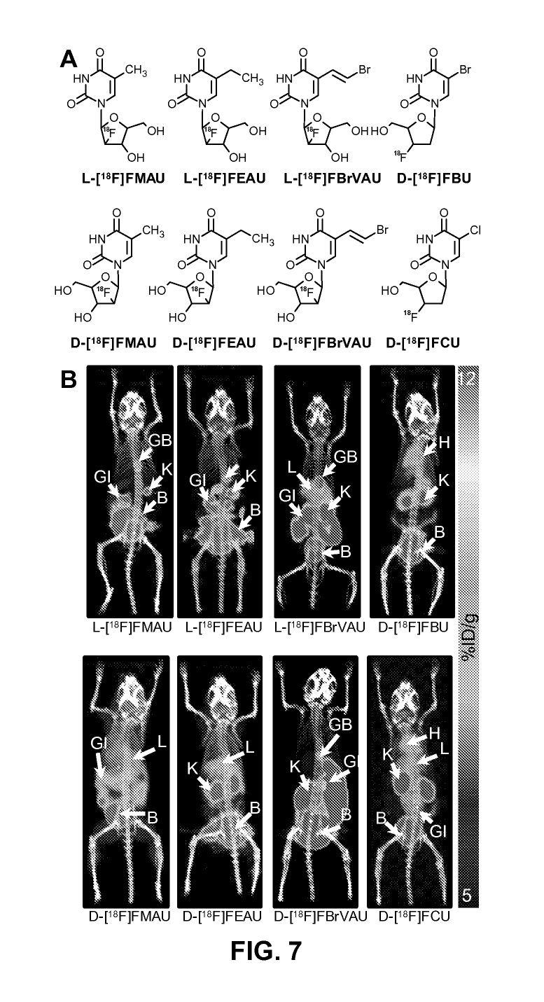

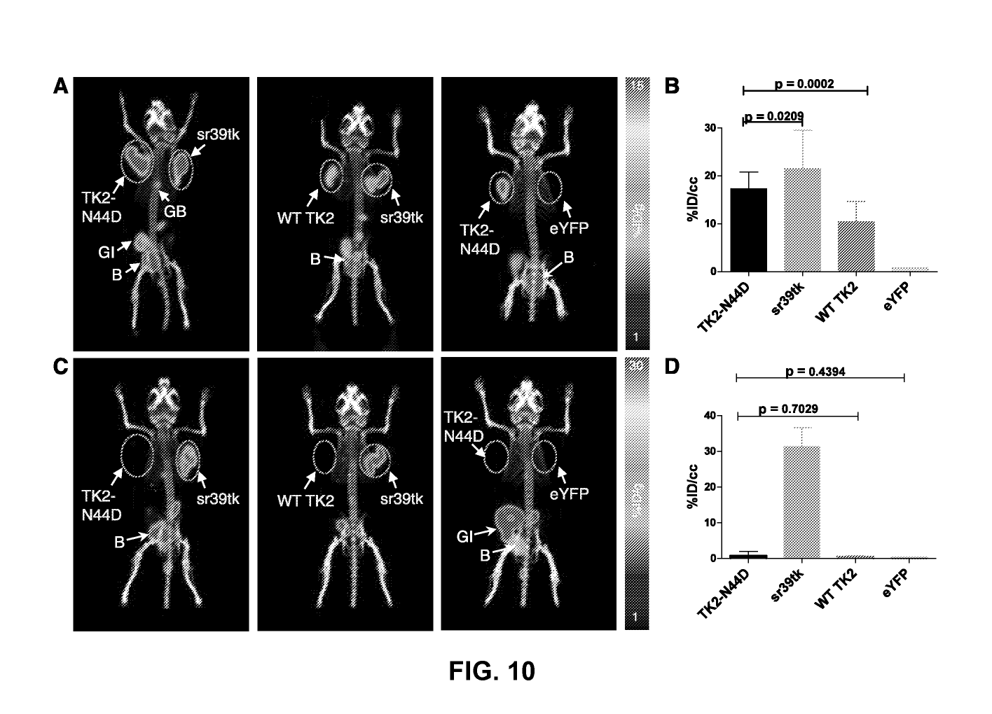

To identify candidate PRPs, eight nucleoside analogs (FIG. 7A) were synthesized that were amenable to ¹⁸F labeling (the first criterion for PRPs). To identify PRP candidates with rapid clearance from normal tissues and low overall background (second criterion for PRGs), biodistribution studies in mice were performed. It was observed that natural (D) nucleosides had higher background than non-natural (L) nucleoside analogs. Amongst the tested candidate probes, L-[¹⁸F]FMAU and L-[¹⁸F]FEAU had the lowest overall background limited to clearance routes such as bladder, gall bladder, and the gastrointestinal (GI) tract (FIG. 7B). Furthermore, increasing the L-[¹⁸F]FMAU uptake time from 1 to 3 hours almost completely eliminated the non-specific GI signals (see FIG. 10). The next step was to identify a human nucleoside kinase that phosphorylates L-[¹⁸F]FMAU and L-[¹⁸F]FEAU, and can be converted into a new PRG that matches four criteria:

1. The protein encoded by the PRG should not cause an immune response against therapeutic cells.

2. To reduce background, the endogenous homolog of the transgenic PRG should not be expressed in cells/tissues of interest. If the endogenous gene is expressed in cells/tissues of interest, it should be localized to a region of the cell that is less accessible to the [¹⁸F]-probe (e.g., mitochondria).

3. The engineered PRG should be amenable to delivery by viral or non-viral vectors.

4. The PRG should be biologically inert. Expression of the nucleoside kinase PRG should not alter cytosolic deoxyribonucleoside triphosphate (dNTP) pools, since this may result in genomic instability (see, e.g. Mathews, C.K. FASEB J 20:1300-1314, 2006) and impaired cell division (see, e.g. Reichard, P. Annu. Rev. Biochem. 57:349-374, 1988). This potential complication is often not considered adequately in developing PRG-PRP systems based on nucleoside kinases.

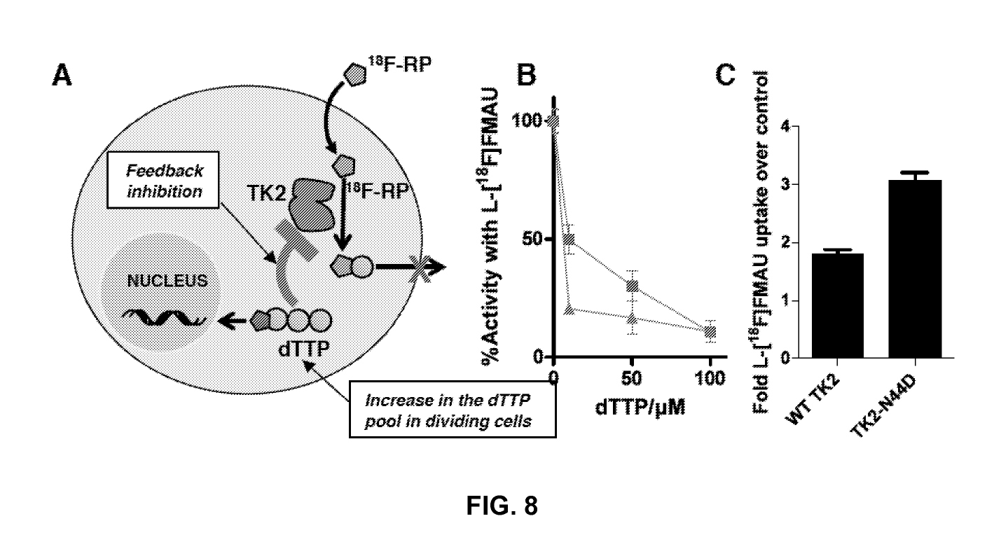

To identify candidate PRGs, the human nucleoside kinases: TK2, dGK, dCK and TK1 were considered (see Table 1 below). These kinases should lack immunogenicity (criterion 1 for PRGs), since they are expressed in human tissues (see, e.g. Amer, E. S., et al. Pharmacol Ther 67:155-186, 1995). As shown in Table 1, arguments can be constructed against and in favor of using any of these kinases as PRGs (see, e.g. Eriksson, S., et al. Cell Mol Life Sci 59, 1327-1346, 2002; Amer, E. S., et al. Pharmacol Ther 67, 155-186, 1995; Wang, J., et al. Biochemistry 38, 16993-16999, 1999; Liu, S. H., et al. Antimicrob Agents Chemother 42, 833-839, 1998; Wang, J., et al. Biochem Pharmacol 59, 1583-1588, 2000; Al-Madhoun, A. S., et al. Mini Rev Med Chem 4, 341-350, 2004; Wang, J., et al. Nucleosides Nucleotides 18, 807-810, 1999). It was reasoned that TK2 (which is normally expressed in the mitochondria—thus matching criterion 2 for PRGs) was the best choice. After truncating the sequence encoding the mitochondrial signal peptide (to target the PRG to the cytosol where it is directly accessible to the PRP), the TK2-based PRG is only 648 base pairs, an optimal size for viral and non-viral vectors (thus matching criterion 3 for PRGs). TK2 has a potential drawback: this kinase is regulated by thymidine triphosphate (dTTP) as part of a protective mechanism against imbalances in mitochondrial dNTP pools (see, e.g. Mikkelsen, N. E., et al. Biochemistry 42:5706-5712, 2003) Inhibition of a TK2-based PRG by dTTP could be problematic when imaging dividing therapeutic cells (e.g. intracellular dNTP pools in proliferating T cells are ˜30-foldhigher than resting lymphocytes (see, e.g. Cohen, A., et al. J Biol Chem 258:12334-12340, 1983) and such elevated levels are sufficient to inhibit TK2 activity, as shown in FIG. 8A).

TABLE 1
Candidate PRGs.

| Human kinase | Advantages | Disadvantages |
|---|---|---|
| thymidine kinase 2 | Broad substrate specificity, | dC and dT (endogenous TK2 |
| (TK2) | active towards L-FMAU, D- | substrates) may compete with the PRP |
| | FEAU, other thymidine | Feedback inhibition by dTTP may |
| | analogs | reduce sensitivity to detect cells with |
| | Low PRP background since | increased cytosolic dNTP pools (such |
| | the endogenous enzyme is | as dividing T cells) |
| | localized in the mitochondria | |
| deoxyguanosine | Low PRP background since | No activity reported with L-FMAU and |
| kinase (dGK) | the endogenous enzyme is | other thymidine analogs |
| | localized in the mitochondria | dA and dG (endogenous dGK |
| | | substrates) may compete with the PRP |
| deoxycytidine kinase | Broad substrate specificity, | Very low activity with L-FMAU |
| (dCK) | some activity reported with | Poor activity with thymidine analogs |
| | L-FMAU | dC, dA and dG (endogenous dCK |
| | Low PRP background due to | substrates) may compete with the PRP |
| | tissue-specific expression of | |
| | the endogenous kinase | |
| thymidine kinase | Some activity with D-FMAU | D-enantiomer selective (thus unable to |
| 1(TK1) | and other thymidine analogs | phosphorylate L-FMAU) |
| | Only one high affinity natural | Feedback inhibition by dTTP may |
| | substrate (dT) may compete | reduce sensitivity |
| | with the PRP in vivo | |

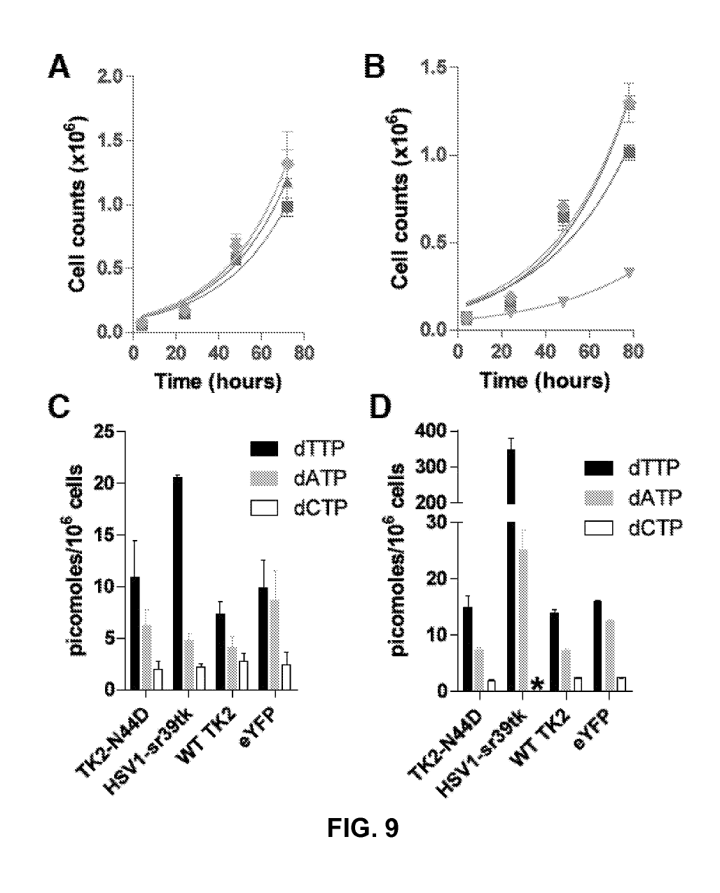

To overcome the negative feedback limitation of TK2, a point mutation-N93D, was engineered. The work done by Piskur and colleagues on the structurally related Drosophila melanogaster deoxyribonucleoside kinase (see, e.g. Welin, M., et al. FEBS J 272:3733-3742, 2005) led one to predict that the N93D mutation would reduce the feedback inhibition of TK2 by dTTP. This prediction was confirmed by a comparison between the wild type (WT) and the TK2-N93D mutant in a kinase assay in which increasing concentrations of dTTP were added to inhibit TK2 enzymatic activity (FIG. 8B). We then determined whether the enhanced resistance to feedback inhibition translates into improved TK2-N93D catalytic activity towards the L-[¹⁸F]FMAU substrate. The murine leukemic cell line L1210 was engineered to express the WT and mutated PRG. PRG-expressing L1210 cell lines were sorted to similar levels of gene expression, using enhanced yellow fluorescent protein (eYFP) as a co-linked fluorescent marker. TK2-N93D transduced cells showed an increased accumulation of L-[¹⁸F]FMAU PRP compared to WT TK2 transduced cells (FIG. 8C). It was also examined whether the expression of TK2-N93D had detrimental effects on cellular physiology (criterion 4 for PRGs). TK2-N93D expression did not affect the growth rate of transduced cells (FIG. 9A) or intracellular dNTP pools (FIG. 9C). In contrast, sr39tk overexpression induced a 4-fold increase in dTTP pools (FIG. 9C). Moreover, when thymidine (dT) was added to the culture media at concentrations normally found in mouse serum, the proliferation of sr39tk+ cells was significantly impaired (FIG. 9B), presumably as a result of significant imbalances in dNTP pools induced by sr39tk overexpression (FIG. 9D). These findings provide evidence that the utility of sr39tk is limited not only by its immunogenicity but also by its effects on normal cellular physiology.

To compare TK2, TK2-N93D and HSV1-sr39tk in vivo, PRG-expressing L1210 cells were implanted subcutaneously in SCID mice that were then scanned with L-[¹⁸F]FMAU (FIG. 10A) and [¹⁸F]FHBG (FIG. 10C) on consecutive days. TK2-N93D showed improved accumulation of L-[¹⁸F]FMAU compared to WT TK2 (FIG. 10B). Signals obtained with sr39tk were slightly higher than those obtained with the human TK2-N93D PRG (the difference was not statistically significant). Neither WT TK2 nor TK2-N93D expressing tumors accumulated detectable amounts of [¹⁸F]FHBG (FIG. 10D). L-[¹⁸F]FMAU accumulation in TK2-N93D transduced tumors was ˜20 times higher than the accumulation in tumors transduced with the control (eYFP only) vector. While this ratio is higher than the 6.67 ratio reported for WT TK2 as a PRG with D-[¹⁸F]FEAU as the PRP (see, e.g. Ponomarev, V., et al. J Nucl Med 48:819-826, 2007), the HSV1-sr39tk/[¹⁸F]FHBG combination still outperforms the TK2-N93D/L-[¹⁸F]FMAU combination by ˜2-fold. The significance of the 2-fold difference for the sensitivity of the new TK2-N93D PRG can be further investigated in the animal models discussed below (see, e.g. Gerth, M. L., et al. Biochem Biophys Res Commun 354:802-807, 2007; Liu, L., et al. Nucleic Acids Res 37:4472-4481, 2009; Iyidogan, P., et al. Biochemistry 47:4711-4720, 2008; Lutz, S., et al. Chimia (Aarau) 63:737-744 2009).

1.2.1. Compare L-[¹⁸F]FEAU and L-[¹⁸F]FMAU in the L1210 Tumor Model.

Although the L-[¹⁸F]FMAU preliminary data is promising, the second candidate PRP, L-[¹⁸F]FEAU was also investigated. The rationale to analyze L-[¹⁸F]FEAU is twofold: a) it is possible that TK2-N93D has a higher affinity for L-FEAU than for L-FMAU—if correct, then L-[¹⁸F]FEAU may be more sensitive; b) for a given PRP, the biodistribution pattern in mice may not predict its biodistribution in humans; it is thus conceivable that human biodistribution studies will reveal that L-[¹⁸F]FEAU is a better PRP than L-[¹⁸F]FMAU. In addition to the cell culture and in vivo studies described in Sect. 1.1 (FIGS. 8-10), one can also compare L-[¹⁸F]FMAU and L-[¹⁸F]FEAU in the adenovirus liver transduction model, as described below.

1.2.2. Evaluate the TK2-N93D/L-[¹⁸]FMAU and L-[¹⁸F]FEAU PRG Systems For Efficacy and Sensitivity Following Adenovirus PRG Vector Delivery to Mouse Liver.

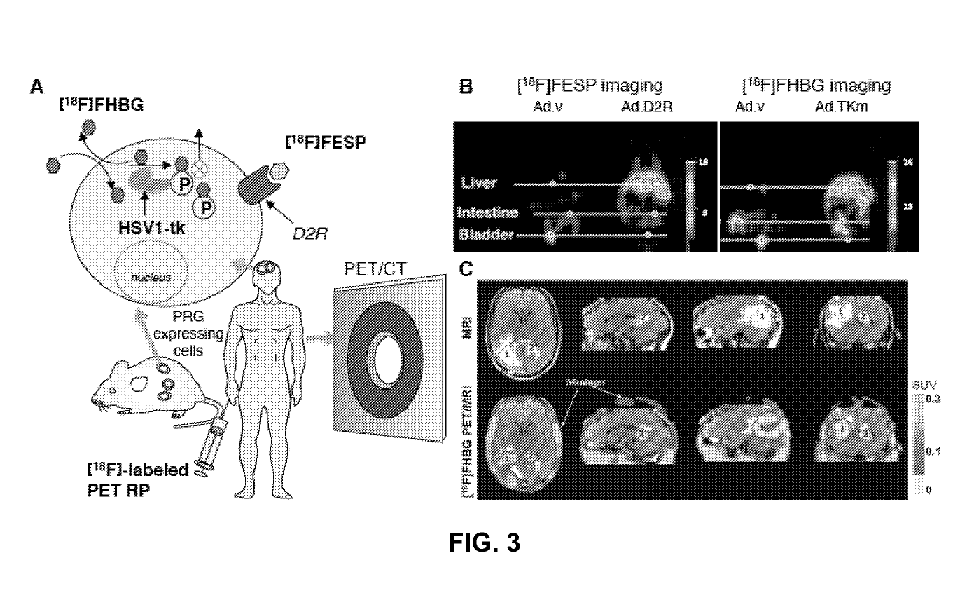

The Ad-liver model described in Sect. 1.2.2. provides the means to rank various PRG-PRP combinations, before testing them in more elaborate hepatic metastases and melanoma targeting strategies. Adenovirus delivery of PRPs to the liver to evaluate PRP-PRG systems eliminates the heterogeneity of target size for PRG delivery encountered with tumors; it also eliminates heterogeneity in target vascularity (a confounding problem for both reporter and probe delivery), and minimizes differences in delivery of therapeutic genes. It is the most reproducible and quantifiable assay with the least number of biological variables. The amount of vector, PRG and target are quantifiable at the end. One can use Ad.CMV.HSV1sr39tk/FHBG as “reference”, and can construct viruses with the identical structure—with the exception that experimental PRGs can be substituted for sr39tk and experimental PRPs can be substituted for FHBG (see FIG. 3B for an example of this approach). To construct and titer the Ad.CMV viruses with alternative PRGs one can use the Invitrogen Gateway cloning system. One can insert the reporter gene into the “entry vector”, and incubate together with the “destination vector” (an Ad5 backbone with the E1 and E3 regions deleted), along with lambda integrase. Identical vectors have been cloned expressing firefly luciferase (fLuc), two modified fLucs, Renilla luciferase (rLuc), a red-shifted rLuc, membrane-bound Gaussia luciferase and GFP, to compare their optical imaging characteristics in the Ad:liver protocol. To compare reference and experimental viruses, groups of three mice can be injected intravenously with 10⁸ Ad.CMV.HSVlsr39tK infectious units (ifu), 10⁸ experimental Ad ifu, 10¹⁰ Ad.CMV.HSVlsr39tK ifu, and 10¹⁰ experimental Ad ifu (see, e.g. Li, H. J., et al. Cancer Res 69, 554-564, 2009, demonstrating that three mice per group can give statistically reliable data). Thus, for each experimental PRG one can run simultaneous, parallel cohorts with Ad.CMV.HSVlsr39tk/FHBG, to provide an internal standard for experimental PRG/PRP comparison. Three days after virus administration, mice can receive 200 μCi of the appropriate PRP (e.g. FHBG, L-[¹⁸F]FMAU, L-[¹⁸F]FEAU), and can be scanned by microPET/CT. Following imaging, the mice can be euthanized and the livers can be homogenized. Ad genomes and murine liver genomes can be measured by qPCR (see, e.g. Li, H. J., et al. Cancer Res 69:554-564, 2009). PRG enzyme assays can also be performed. Non-invasive PRG-PRP imaging data and in vitro enzyme activities can be normalized to viral genomes/liver and compared for the reference and experimental PRGs. Using a low (10⁸ ifu) and high (10¹⁰ ifu) dose of PRG-expressing vector can give us an initial estimate of sensitivity and dynamic range for each experimental PRG-PRP system. This protocol can also provide a simultaneous internal comparison of biodistribution and tissue background for L-[¹⁸F]FMAU and L-[¹⁸F]FEAU, relative to FHBG.

1.2.3. Analyze the Potential Immunogenicity of the TK2-N93D PRG.

Although huTK2-N93D closely resembles the endogenous TK2 protein and will therefore be less immunogenic in humans than viral TK polypeptides, it cannot be assumed that this PRG completely lacks immunogenicity in humans. Not only the N93D mutation may render TK2-N93D immunogenic but also its overexpression may break immunological tolerance. The immunogenicity of an intracellular protein (such as TK2-N93D) is primarily determined by the presentation of short antigenic peptides on the cell surface in the context of Major Histocompatibility Complex (MHC) class I molecules and by the presence in the host immune repertoire of CD8+ T cells that express a T cell receptor (TCR) able to recognize the peptide-MHC class I complexes. Abolishing peptide-MHC presentation should eliminate the immunogenicity of any given protein. One can use in silico methods to determine the epitopes contained within the TK2-N93D PRG that can be presented by host MHC class I molecules. Several algorithms are available to obtain this information: SYFPEITHI (see, e.g. Rammensee, H., et al. Immunogenetics 50:213-219, 1999), NetMHC (see, e.g. Lundegaard, C., et al. Nucleic Acids Res 36:W509-512, 2008), NIH BIMAS (see, e.g. Parker, K. C., et al. J Immunol 152:163-175, 1994) and Rankpep (see, e.g. Reche, P. A., et al. Hum Immunol 63:701-709, 2002). One can use the consensus values obtained from these algorithms to identify all the potential HLA-A2-binding epitopes encoded by TK2-N93D. Given its predominance in humans, one focuses on the HLA-A2 allele; however, similar approaches are applicable to all human or mouse MHC haplotypes. A fluorescence-polarization assay (see, e.g. Bakker, A. H., et al. Proc Natl Acad Sci USA 105:3825-3830, 2008) can be used to validate the binding affinity for HLA-A2 of the in silico predicted binders. One can then design mutations in the identified TK2 encoded epitopes that can abolish their binding to HLA-A2 (and therefore eliminate the immunogenicity of TK2-N93D). The affinity of the mutants for L-[¹⁸F]FMAU and L-[¹⁸F]FEAU and their efficacy as PRGs in vivo in the Ad:liver model can be determined as described in Sect. 1.2.2.

### 1.3. Potential Caveats and Alternative Approaches.

The feasibility of this approach is supported by preliminary data. Most of the remaining steps to realize this goal involve either taking a second candidate probe (L-[¹⁸F]FEAU) through an identical validation algorithm as that described for L-[¹⁸F]FMAU or, as shown, for FHBG (in the adenovirus liver transduction model) (see, e.g. Garcia, K. C., et al. Proc Natl Acad Sci USA 98:6818-6823, 2001; Anderton, S. M., et al. J Exp Med 193:1-11, 2001; Radu, C. G., et al. Int Immunol 12:1553-1560, 2000; Radu, C. G., et al. J Immunol 160:5915-5921, 1998).

### Example 2

### Illustrative Preclinical Studies Evaluating PRG-PRP Systems

The best PRG-PRP candidates can be tested in a hepatic colorectal cancer (CRC) model (see, e.g. Li, H. J., et al. Cancer Res 69:554-564, 2009) to mimic a clinical application of viral vectors for tumor imaging and therapy. This can be carried out using a murine model of combined gene and cell immunotherapy against melanoma that is directly relevant to ongoing cancer immunotherapy clinical trials.

Investigating the sensitivity and specificity of the hTK2-N93D/L-[¹⁸F]FMAU and hTK2N93D/L-[¹⁸F]FEAU PRG-PRP Systems in Murine Models of Cancer Therapy.

The models one can use in such studies include (1) gene therapy of hepatic metastases of colorectal cancer and (2) T-cell immunotherapy of melanoma. These cancer therapy models are well established in laboratories and are used extensively in academia and industry.

### Evaluating New PRG-PRP Systems for Efficacy and Sensitivity Following Ad PRG Vector Delivery to Hepatic Colorectal Cancer (CRC) Metastases.

One can use Ad vectors in which the sr39tk PRG and the experimental PRGs are driven by the human Cox2 promoter. Because COX-2 is expressed in CRCs and not expressed in liver (see, e.g. Fujita, T., et al. Cancer Res 58:4823-4826, 1998; Ishikawa, T. O., et al. Mol Imaging Biol 8:171-187, 2006), Cox2 “transcriptional restriction” results in an ˜24 fold difference in reporter gene expression in CRC metastases versus liver (see, e.g. Li, H. J., et al. Cancer Res 69:554-564, 2009). Human LS174T (rLuc) CRC cells (10⁶) can be injected into the upper left liver lobe of nu/nu mice (see, e.g. Li, H. J., et al. Cancer Res 67:5354-5361, 2007). One can monitor tumor burden weekly by optical imaging (see, e.g. Liang, Q., et al. Mol Imaging Biol 6:385-394, 2004), using the rLuc substrate coelenterazine. Tumor burden is reproducible and easily measurable at three weeks (see, e.g. Li, H. J., et al. Cancer Res 69:554-564, 2009). At this time, groups of three mice can be injected with 10⁸ Ad.Cox2HSVlsr39tk ifu, 10⁸ experimental-PRG Ad ifu, 10¹⁰ Ad.Cox2HSVlsr39tk ifu or 10¹⁰ experimental-PRG Ad ifu. Three days later, mice can be injected with the appropriate PRP and imaged by microPET/CT. The following day mice can be imaged with [¹⁸F]FDG. [¹⁸F]PRP:[¹⁸F]FDG hepatic retention ratios can provide a non-invasive analysis of the sensitivity, and provide an indication of the dynamic range, of the experimental PRG-PRP system versus the HSVlsr39tk/FHBG system. After imaging, mice can be euthanized, and PRG enzyme activities in liver homogenates can be assayed. Adenovirus genomes, human genomes (to measure the tumor burden) and murine liver genomes (to normalize data) can be monitored by PCR (see, e.g. Li, H. J., et al. Cancer Res 69:554-564, 2009). These data will provide direct and quantitative comparisons of the sensitivity of experimental PRG-PRP systems in detecting colorectal cancer liver metastases. The data on relative efficacy and sensitivity of alternate PRG-PRP systems can be extrapolated to any PRG delivery system.

Evaluating New PRG-PRP Systems in a Murine Model of Combined Cell and Gene Therapy Against Melanoma.

The Pmel-1/B16 model (see, e.g. Overwijk, W. W., et al. J Exp Med 198:569-580, 2003) is extensively used by many groups working in the field of tumor immunology (see, e.g. Finkelstein, S. E., et al. J Leukoc Biol 76:333-337, 2004)). It closely resembles the ACGT clinical procedure shown in FIG. 2. Rejection of established murine B16 melanoma tumors is achieved by a combined immunotherapy protocol using CD8+ T cells (obtained from the Pmel-1 transgenic mice and specific for the self/tumor antigen gp100 present on B16 cells) (see, e.g. Ponomarev, V., et al. J Nucl Med 48, 819-826, 2007; Likar, Y., et al. J Nucl Med 51, 1395-1403, 2010; Doubrovin, M. M., et al. Cancer Res 67, 11959-11969, 2007; Li, H. J., et al. Cancer Res 69, 554-564, 2009; Mathews, C. K. FASEB J 20, 1300-1314, 2006; Reichard, P. Annu. Rev. Biochem. 57, 349-374, 1988; Amer, E. S., et al. Pharmacol Ther 67, 155-186, 1995; Mikkelsen, N. E., et al. Biochemistry 42, 5706-5712, 2003; Cohen, A., et al. J Biol Chem 258, 12334-12340, 1983). T cells are adoptively transferred in tumor bearing mice, which are also treated with lymphodepletion (500 cGy), dendritic cell (DC) vaccination and high dose systemic IL-2 administration (see, e.g. Liu, S., et al. Int Immunol 19:1213-1221, 2007; Liu, Y. L., et al. Journal of the Formosan Medical Association=Taiwan yi zhi 108:587-591, 2009; Overwijk, W. W., et al. J Exp Med 188:277-286, 1998; Sikora, A. G., et al. J Immunol 182:7398-7407, 2009). In a previous study, CD8+ T Pmel-1 T cells were transduced with a multicistronic retroviral vector encoding sr39tk and eYFP (to allow ex vivo detection of transduced cells by FACS) (see, e.g. Shu, C. J., et al. Int Immunol 21:155-165, 2009). Transduced cells were adoptively transferred in lymphodepleted BL/6 mice bearing B16 melanoma. Recipient mice were serially imaged by [¹⁸F]FHBG microPET/CT. In vivo quantification of [¹⁸F]FHBG accumulation in spleen and lymph nodes correlated with numbers of sr39tk+eYFP⁺ T cells present at these sites (measured ex vivo by FACS). The lower limit of detection for PRG imaging in this model was ˜1×10⁴ sr39tk+ T cells in a lymph node with a volume of 1-2 mm³. These findings provide evidence that the PRG-PRP technology can be used to detect populations of cells that represent less than 5% of the total number of cells in the imaged lymph node. One can use the Pmel-1/B16 ACGT model to determine whether one can achieve the same, if not better, lower limit of detection as that obtained with sr39tk and FHBG using the new, non-immunogenic PRG systems. Based on previous work, it is estimated that 4-6 mice per group will be needed to obtain statistically significant values.

2.3. Potential Caveats and Alternative Approaches.

Since Ad-CRC liver metastasis and melanoma cell-based immunotherapy animal models are routinely used in the laboratories, one does not anticipate any difficulties. It is possible however that data from these models will indicate that new PRG-PRP systems are less sensitive than the current HSV1-sr39tk/FHBG gold standard. Due to this possibility, one can collaborate with other laboratories to identify new TK2 mutants with improved catalytic activity towards L-FMAU and L-FEAU.

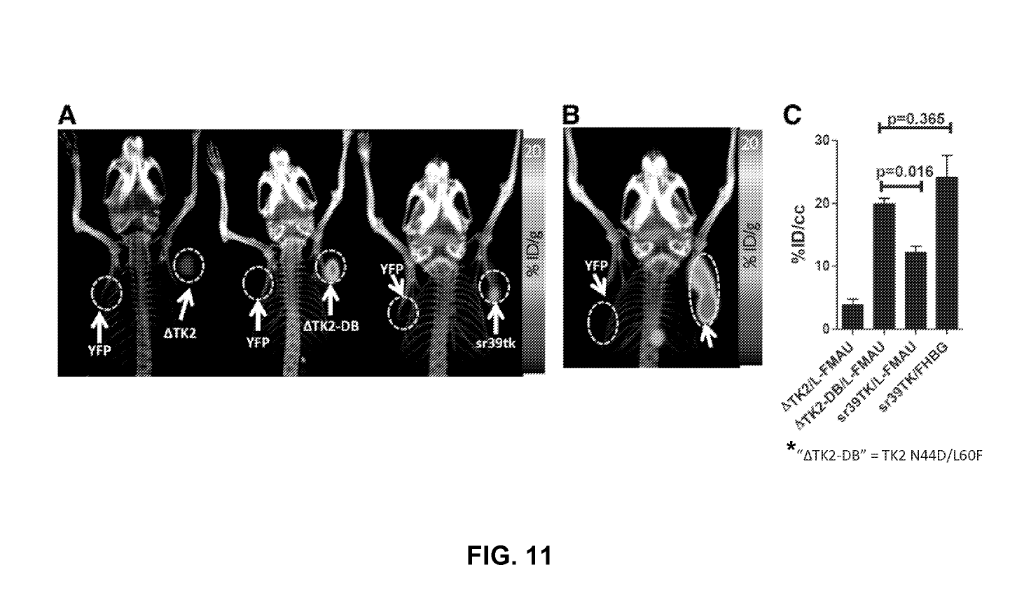

As yet another contingency plan, collaborative studies with the structure of nucleoside kinases have also been initiated. A number of new human TK2 mutants have been identified for improving the affinity and specificity for ¹⁸F-L-FMAU. The rationale behind the design of these new mutants was to improve the affinity of TK2 for L-nucleosides at the expense of the endogenous (natural) substrates that are in the D-nucleoside configuration. The new mutants suggested on both the wild type and N93D TK2 backbone have been generated. It has been found that one of the double mutants (TK2 N93D/L109F, referred to as “ΔTK2-DB” in FIG. 11) is significantly more sensitive than N93D TK2 (FIG. 11). This finding confirms that improvements of the hTK2 N93D system are possible.

### Example 3

### Products for Labeling and Long Term Cell-Tracking PET Studies

In embodiments of the invention, aspects of the technology can be translated as commercialized kits that can be used in labeling and long term cell-tracking PET studies. The products can be for preclinical animal model research; products can be disseminated for clinical use once they gain FDA approvals. ELIXYS™, an automated, modular radiochemistry platform can be obtained to produce and deliver L-[¹⁸F]FMAU and L-[¹⁸F]FEAU to other groups for use in preclinical therapeutic gene and cell tracking studies.

Develop, Validate, and Commercialize Kits for PRG Delivery into Murine and Human Therapeutic Cells and Disseminate this New Capability to Wider Communities of End-Users.

Kits can use both lentiviral and non-viral ΦC31 integrase technologies to obtain stable PRG expression. ELIXYS™, Sofie Biosciences'automated, modular radiochemistry platform that enables the synthesis of these probes with high yield and high specific activity, will be obtained to complement the kits with the L-[¹⁸F]FMAU or L-[¹⁸F]FEAU PRPs.

To take full advantage of PET reporter gene imaging technology, the PRG should be delivered such that it is stably expressed in the original and in all progeny cells, via chromosomal integration, following delivery. Given the risk of genotoxicity by random genomic integration of most viral gene delivery approaches, non-viral strategies are preferred for clinical applications. However, viral strategies are more robust and are still widely used in gene therapy (see, e.g. Chowdhury, E. H. Expert Opin Drug Deliv 6:697-703, 2009). One can incorporate the non-immunogenic PRGs developed in Examples 1 and 2 into viral and non-viral vectors, to create the essential components of Ready-To-Use kits for permanent labeling of TCs. This can be divided into three subaims:
Production of PRG lentiviral delivery vectors;
Development of PRG non-viral delivery vectors;
Studies to determine conversion factors to quantify in vivo TCs genetically marked with the new PET reporter genes.

Produce PRG Lentiviral Delivery Vectors.

Lentiviral vectors infect both dividing and non-dividing cells and integrate stably into the genome, favoring introns over exons (see, e.g. Pluta, K. et al. Acta Biochimica Polonica 56:531-595, 2009). Table 2 below describes a plan to develop lentivector-based PRG delivery kits (see, e.g. Pluta, K., et al. Acta Biochimica Polonica 56, 531-595, 2009; De, A., et al. Gene Therapy Protocols: Production and In Vivo Applications of Gene Transfer Vectors, Vol. 1 (ed. Le Doux, J. M.) 177-202, Humana Press, Totowa, 2008; Loebinger, M. R., et al. Thorax 65, 362-369, 2010; Motaln, H., et al. Cancer 116, 2519-2530, 2010; Kode, J. A., et al. Cytotherapy 11, 377-391, 2009; Yaghoubi, S. S., et al. Nat Protoc 1, 2137-2142, 2006; Chalberg, T. W., et al. Journal of Molecular Biology 357, 28-48, 2006; Keravala, A., et al. J. Neurosci. Methods 173, 299-305, 2008; Wu, J. C., et al. Physiol Genomics 25, 29-38, 2006; Menon, L. G., et al. Stem Cells 25, 520-528, 2007; Yaghoubi, S. S., et al. Nat Protoc 1, 3069-3075, 2007). The final products are four Ready-To-Use viral PRG cell-labeling kits: 1) lentivirus, pseudotyped for mouse T cells carrying TK2-N93D or other tk2 mutants; 2) lentivirus, pseudotyped for hMSCs, carrying TK2-N93D or other tk2 mutants; 3) lentivirus pseudotyped, for mouse T cells, carrying HSV1-sr39tk; 4) lentivirus, pseudotyped for hMSCs, carrying HSV1-sr39tk. Kits can include all reagents and optimized protocols for mouse CD8⁺ T cell or hMSC transduction.

TABLE 2
Product development strategy

| | Viral PRG Delivery Kits | Non-Viral PRG Delivery Kits |
|---|---|---|
| | Lentivirus | ΦC31 Integrase and Nucleofection |
| Synthesis of | Two types of replication incompetent | Plasmids with ΦC31 recognition |
| critical kit | lentiviral transgene delivery vectors | sequences (attB), a PRG and an |
| components | containing an antibiotic selection | antibiotic selection marker |
| | marker, each pseudotyped for | CellSight has obtained the pCMV-Int |
| | efficient and non-toxic transduction of | plasmid. |
| | mouse T cells and hMSCs. | |
| Cancer therapy | Mouse CD8+ CTLs (see ACGT model in Sect. 2.2); hMSCs (preferentially | |
| models for | migrate to and survive in several human tumor xenografts in mice. Easily | |
| validation | extracted from multiple tissue sources and expandable in culture. Useful for | |
| | delivery of several anticancer agents) | |
| Transgene | Lentiviral transduction. Antibiotic | Co-nucleofection of pattB-PRG and |
| Delivery | selection of successfully transduced | pCMV-Int into therapeutic cells using |
| | cells. | Lonza's optimized nucleofection |
| | | reagents for each cell type. Antibiotic |
| | | selection of successfully PRG |
| | | integrated cells. |
| Confirm | PRG expressions will be analyzed through variations of enzyme and probe | |
| continuous | uptake assays described by Yaghoubi, et al. | |
| stable PRG | PRG expression measured twice a week following transduction or co- | |
| expression | nucleofection of mouse CTLs for as long as they can be maintained in | |
| | culture and of hMSCs for 10 weeks. | |
| Analysis of PRG | Not Applicable | Sites of chromosomal integration will |
| integration site | | be identified according to established |
| | | PCR procedures |

| | |
|---|---|
| Quality | Test effects on growth and proliferation, on normal expression of |
| Assurance | endogenous genes assessed by gene microarray analysis as described |
| | previously; for hMSCs, assess differentiation ability |
| | Mouse CTLs: the ACGT preclinical model described in Sect. 2.2. |
| | Human MSCs: Tumor homing of PRG expressing hMSCs studied in a colon |
| | tumor xenograft model. MicroPET scans performed as described by |
| | Yaghoubi et al. to monitor cell trafficking |

Although lentiviral vectors can achieve stable transgene expression in most TCs, their random integration, possible production of replication competent viruses, regulatory concerns and the high cost of GMP grade lentivirus may limit their utility in certain clinical trials. The ΦC31 integrase enzyme (encoded by a Streptomyces soil bacteria phage) catalyzes site-specific chromosomal transgene integration following plasmid delivery (see, e.g. Keravala, A., et al. (eds. Davis, G. & Kayser, K. J.) Chromosomal Mutagenesis, Vol. 435:165-173, Humana Press Inc., Totowa, N. J., 2008; Ginsburg, D. S., et al. Advances in Genetics 54:179-187, 2005). ΦC31 integrase is functional in mammalian cells (see, e.g. Calos, M. P. Current Gene Therapy 6:633-645, 2006).

The enzyme recognizes two ˜30 base pair sequences, attB and attP. When plasmids carrying both attB and ΦC31 integrase (pCMV-Int) are introduced into mammalian cells, the enzyme carries out sequence-specific recombination with chromosomal sequences (pseudo attP sites) that resemble attP. Approximately 100 potential integration sites, have been identified, most of which are intergenic and none are near known cancer genes; thus oncogene activation is unlikely (see, e.g. Chalberg, T. W., et al. Journal of Molecular Biology 357:28-48, 2006). ΦC31 integrase typically generates only one integration event per cell (see, e.g. Chalberg, T. W., et al. Journal of Molecular Biology 357:28-48, 2006). Studies have demonstrated integration and expression of multiple genes carried on a single plasmid (see, e.g. Calos, M. P. Current Gene Therapy 6:633-645, 2006). Table 2 describes a proposed plan for developing non-viral PRG delivery kits. The final products are four Ready-To-Use ΦC31 integrase-based PRG cell-labeling kits containing the two essential plasmids, nucleofection reagents, and optimized protocols. These kits can allow delivery of either TK2-N93D or sr39tk to mouse T cells and to hMSCs.

Determine Conversion Factors to Estimate the Numbers of Therapeutic Cells In Vivo, Using PET Reporter Gene Imaging.

Increasing numbers (5, 10, 50, 100, 200, 500, and 800×10³) of mouse CD8⁺ T cells or hMSCs with stably integrated PRGs can be injected into colon cancer tumor xenografts. One recognizes that direct injections into tumor lesions lack clinical relevance. However, this approach is only intended for the initial determination of conversion factors, which can then be validated in the clinically relevant models described above. PRG expression per cell can be determined immediately prior to injection. Four hours after TC injection, mice can receive L-[¹⁸F]FMAU and can be scanned three hours after tracer injection. L-[¹⁸F]FMAU percent injected dose (% ID) can be measured within an ROI drawn over the entire xenograft; % ID/tumor can be calculated. One can use >3 mice per group to obtain statistically significant values. These measurements can be related to cell numbers and normalized by cell expression level, to obtain conversion factors to estimate TC numbers present in tumor xenografts, based on L-[¹⁸F]FMAU/FEAU signal intensity.

3.4. Commercialization Strategy.

The kits can be marketed in two ways: (i) selling kits to investigators who wish to label TCs for pre-clinical studies. Quality control data (Table 2) and calibration studies (Sect. 4.2.3.3) can be published and also provided on a website to help customers decide whether the kits are appropriate for their cell therapy applications and offer better alternatives to other options for TC PK monitoring; (ii) establish service contracts and use the kits technologies to custom-prepare PRG-labeled TCs for clients that want to monitor the PK of their cells. The contracts, can—at the client's option—include additional services, such as quality assurances for specific PRG labeled TCs, determination of integration sites, PET imaging in small or large research animal models and PET image data analysis to estimate cell quantities. To perform PET imaging services for its customers, ELYXIS™, an automated modular radiochemistry device developed by Sofie Biosciences can be purchased. The PRPs used to generate the preliminary data shown in FIGS. 8-11 have all been synthesized using this prototype.

### Example 4

### Biodistribution and Dosimetry of L-[¹⁸F]FMAU and L-[¹⁸F]FEAU in Healthy Volunteers

Data can be generated for the eIND submission. One can provide a study coordinator, recruit healthy volunteers in accordance with FDA requirements, ensure the study follows the FDA approved protocol, make protocol amendments as needed, process the safety data, report necessary protocol deviations, and report any adverse effects.

Obtain Exploratory Investigational New Drug (eIND) Approval to Test the New PRPs in Humans.

An eIND application can be submitted to the FDA to enable clinical evaluation of L-[¹⁸F]FMAU and L-[¹⁸F]FEAU. One can then determine the biodistribution and dosimetry of these PRPs in healthy volunteers. These “first-in-human” studies can set the stage for a follow-up study in which a full IND application can be submitted to the FDA to initiate clinical testing of the new PRG-PRP systems in cancer patients.

The FDA eIND regulatory process allows microdosing studies ( 1/100^{th} of the dose calculated to yield a pharmacologic effect) in small numbers of human subjects during early phase 1 (phase “0”) clinical trials. The steps required for submitting of an eIND application are: 1) preclinical safety/toxicity; 2) preclinical dosimetry; 3) chemistry, manufacturing and quality controls and 4) clinical protocols.

4.1. Preclinical Safety/Toxicity Studies:

The projected mass dose of L-[¹⁸F]FMAU and L-[¹⁸F]FEAU is ˜4 μg single dose. This dose is 2500-fold lower than that used as the lowest pharmacological L-FMAU (Clevudine, 10 mg/day for a minimum of 28 days (see, e.g. Marcellin, P., et al. 40:140-148, 2004)). Given the magnitude of this difference, any pharmacological effects or toxicity are highly improbable, both for L-FMAU and for the related compound L-FEAU. To obtain eIND approval from the FDA one can perform additional toxicity studies in rats. Rats can be administered a dose 100× higher than those given for imaging in humans on day 0 and monitored over a period of 14 days. Any side effects or organ damage can be determined by clinical chemistry, necropsy and histology on days −1, +1, +7 and +14.

4.2. Dosimetry Studies.

The radiation safety of the probes can also be evaluated by doing a dosimetry analysis in mice, as previously described (see, e.g. Yaghoubi, S. S., et al. J Nucl Med 47:706-715, 2006). The biodistribution of the PRPs can be studied in healthy human subjects. Tracer concentrations in all organs can be quantified non-invasively. Similar biodistribution/dosimetry clinical studies with the FAC imaging probes have been conducted.

4.3. Chemistry, Manufacturing, Controls and eIND Submission.

Briefly, F-18 ions are generated by a cyclotron particle accelerator, transferred to a “hot cell” and dried to remove water. The radiosynthesis is carried out and the probe product is purified by preparative HPLC, analyzed by HPLC and GC, and sterile filtered into multi-dose sterile vials. One can follow FDA recommendations that specify safety considerations for diagnostic radiopharmaceuticals including: verification of the mass dose of the radiolabeled and unlabeled moiety; assessment of the mass, and toxic potency; assessment of potential pharmacologic or physiologic effects due to molecules that bind with enzymes; and evaluation of all components in the final formulation for toxicity (e.g., excipients, reducing drugs, stabilizers, anti-oxidants, chelators, impurities, and residual solvents).

Upon completion of pre-clinical safety studies described above, a pre-eIND meeting can be scheduled with the FDA to review the submitted results and address potential questions. Following that meeting, one can submit an eIND for L-[¹⁸F]FMAU and L-[¹⁸F]FEAU. Within 30 days of eIND submission, the phase “0” clinical trials can be initiated.

4.4. Potential Caveats and Alternative Approaches.

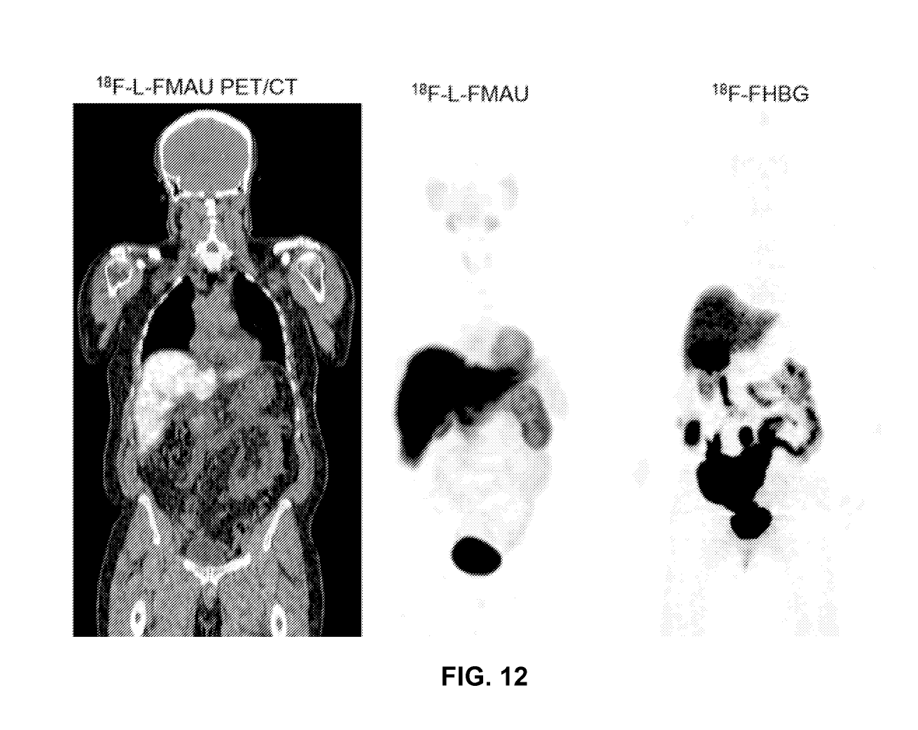

It is possible that the biodistribution of the L-[¹⁸F]FMAU PET reporter gene probe in humans might be less favorable than what has been observed in mice. It was critically important to address this potential caveat before proceeding with the eIND application. Thus, approval has been obtained to perform the first ever L-[¹⁸F]FMAU whole-body PET/CT imaging in a healthy human volunteer. The image (FIG. 12) is extremely promising, since, compared to FHBG, L-[¹⁸F]FMAU shows very low tracer background within the abdomen and pelvis. This is a significant advantage over FHBG, which has high abdominal background in both mice and humans. It has also been learned that cell trafficking to liver and heart might be difficult to monitor using L-[¹⁸F]FMAU (since this probe accumulates in these tissues even in healthy volunteers). One can evaluate other proposed PET reporter gene probe (L-[¹⁸F]FEAU) for applications that require imaging of cell trafficking to liver and heart.

4.5. Additional Contingency Plans.

Recent studies show that increased retention of D-[¹⁸F]FMAU in human tumors reflects trapping of this probe by endogenous TK2 during mitochondrial stress (see, e.g. Tehrani, O, S., et al. European journal of nuclear medicine and molecular imaging 35:1480-1488, 2008). L-[¹⁸F]FMAU uptake may also increase in tumors that experience mitochondrial stress and this may interfere with the detection at these sites of therapeutic cells genetically labeled with the new mutant TK2 reporter genes. One can use previously described experimental approaches (see, e.g. Tehrani, O, S., et al. European journal of nuclear medicine and molecular imaging 35:1480-1488, 2008) to compare under conditions of mitochondrial stress the uptake of D-[¹⁸F]FMAU and L-[¹⁸F]FMAU by cells that express the mutant TK2 PRGs or vector (eYFP only) control. One expects that overexpression of a PRG optimized for L-[¹⁸F]FMAU and engineered to localize in the cytosol will allow these cells to accumulate much higher amounts of L-[¹⁸F]FMAU than those accumulated by control cells via the expression of endogenous TK2. If this is not the case, one can focus on L-[¹⁸F]FEAU, the other candidate PET reporter gene described in this application. Compared to D-[¹⁸F]FMAU and L-[¹⁸F]FMAU, L-[¹⁸F]FEAU is expected to have much lower affinity for endogenous human TK2 and thus its uptake in tumors should be insensitive to mitochondrial stress.

4.6 Future Directions.

Future directions include evaluating ΔTK2-DB/L-FMAU in a murine ACT model and in a model of gene therapy; solving the crystal structure of TK2 with L-FMAU; further optimization of ΔTK2 reporter genes; and evaluation of L-FEAU/L-FPAU as reporter probes.

### Example 5

### Evaluation of ¹⁸F-L-FMAU, a Novel Positron Emission Tomography Reporter Probe in Mice and Humans'

Gene and cell based therapies hold the promise of curing a variety of incurable diseases if therapeutic transgene (TG) and therapeutic cell (TC) pharmacokinetic issues hampering their progress can be resolved. Radionuclide-based imaging reporter gene (IRG) systems are currently the only IRG systems sensitive enough for general and non-invasive monitoring of TG and TC kinetics in humans. A variety of positron emission tomography (PET) IRGs (PRGs) have been developed, but none are yet ideal TG or TC kinetics imaging tools. A new development strategy is pursued which began by evaluating the biodistribution of several candidate fluorine-18 radiolabeled PET tracers in vivo to identify the most suitable PET reporter probe (PRP) for a class of potentially non-immunogenic human derived PRGs.

5.1 Method.

Initially, a group of nucleoside analogs amenable to fluorine-18 labeling were identified. These PET tracers, 5 of which were novel, were then screened to determine their biodistribution in C57/BL6 mice (n=3 for each PET tracer) through dynamic microPET scans. Whole-body clinical PET scans were performed in a healthy male human volunteer at 4 time points for up to 2.5 hours to determine tissue time activities of the top candidate, 1-(2′-[¹⁸F]fluoro-5-methyl-β-L-arabinofuranosyl)uracil ([¹⁸F]L-FMAU). Rational design is utilized to introduce mutations into human nucleoside kinase thymidine kinase 2 (TK2) to improve the affinity of the kinase for the top candidate probe. The sensitivity and specificity of the novel PET reporter probe/gene pair was then determined in vitro and in vivo using a murine cancer model.

5.2 Results.

Of the 8 PET tracers synthesized, 4 exhibited lower abdominal background than 9-[4-[¹⁸F]fluoro-3-(hydroxymethyl)butyl]guanine ([¹⁸F]FHBG), the only PRP that has thus far been used for imaging TCs in patients. Of these four probes, ¹⁸F-L-FMAU was selected as the top candidate based on its biodistribution in mice and the fact that a compound with the same chemical structure had already been investigated in humans, facilitating relatively rapid translation into clinical studies. Whole-body ¹⁸F-L-FMAU PET scans in the healthy human volunteer showed that it had lower intestinal background than [¹⁸F]FHBG, indicating ¹⁸F-L-FMAU may be more suitable for imaging TG and TC kinetics in the lower abdomen of patients. A TK2 point mutant (TK2-N0, referring to the TK2-N93D mutant) is designed that showed a two-fold increase in in vivo uptake of ¹⁸F-L-FMAU compared to TK2 when assayed in a murine xenograft cancer model. A second TK2 mutant (TK2-N5, referring to the TK2-N93D/L109F mutant) was also identified that showed a two-fold increase in in vitro uptake of ¹⁸F-LFMAU as well as less resistance to inhibition by thymidine compared to TK2-N0.

5.3 Conclusions.

Using a novel platform for the development of PRG/PRP systems, ¹⁸F-L-FMAU has been identified as a suitable PRP for imaging mutant human tk2 PRGs. Paired with the novel PET reporter gene, TK2-N5, this can expand the utility of PET reporter gene systems in pre-clinical systems and potentially in clinical applications.

### Example 6

### Illustrative Use of Positron Emission Tomography (PET) Reporter Gene Imaging to Non-Invasively Monitor Cell-Based Therapies

Positron emission tomography (PET) reporter gene imaging can be used to non-invasively monitor cell-based therapies. Therapeutic cells engineered to express a PET reporter gene (PRG) specifically accumulate a PET reporter probe (PRP) and can be detected by PET imaging. Expanding the utility of this technology requires the development of new non-immunogenic PRGs. Here, a new PRG-PRP system is described that employs, as the PRG, a mutated form of human thymidine kinase 2 (TK2) and 2′-deoxy-2′-¹⁸F-5-methyl-1-β-L-arabinofuranosyluracil (L-¹⁸F-FMAU) as the PRP. L-¹⁸F-FMAU was identified as a candidate PRP and its biodistribution was determined in mice and humans. Using structure-guided enzyme engineering, a TK2 double mutant (TK2-N93D/L109F) was generated that efficiently phosphorylates L-¹⁸F-FMAU. The N93D/L109F TK2 mutant has lower activity for the endogenous nucleosides thymidine and deoxycytidine than wild type TK2, and its ectopic expression in therapeutic cells is not expected to alter nucleotide metabolism. Imaging studies in mice indicate that the sensitivity of the new human TK2-N93D/L109F PRG is comparable with that of a widely used PRG based on the herpes simplex virus 1 thymidine kinase. These findings provide evidence that the TK2-N93D/L109F/L-¹⁸F-FMAU PRG-PRP system is useful in preclinical and clinical applications of cell-based therapies.

The inability to routinely monitor the tissue pharmacokinetics of therapeutic genes and cells and correlate this information with therapeutic outcomes represents a significant roadblock in the clinical adoption of these emerging therapies. Most cell/gene therapy trials use invasive biopsy techniques to localize therapeutic genes or therapeutic cells at target sites. However, invasive techniques are prone to sampling errors and carry risks for the patients. There is an unmet need for techniques to monitor the whole-body tissue distribution of therapeutic cells and therapeutic genes, to quantify therapeutic cells, and to measure therapeutic gene expression at all locations non-invasively and sequentially after treatment.

This unmet need can be addressed by PET3 reporter gene (PRG) imaging (see, e.g. Herschman, H. R. (2004) Crit. Rev. Oncol. Hematol. 51, 191-204). A PRG encodes a protein that mediates the specific accumulation of a PET reporter probe (PRP) labeled with a positron-emitting isotope (see, e.g. Gambhir, S. S., and Yaghoubi, S. S. (eds) (2010) Molecular Imaging With Reporter Genes, pp. 258-274, Cambridge University Press, Cambridge, UK). Such non-invasive PET measurements may predict and/or evaluate treatment efficacy and the risk of side effects; they can provide information that complements data obtained using invasive techniques, such as serial biopsies (see, e.g. Gambhir, S. S., and Yaghoubi, S. S. (eds) (2010) Molecular Imaging With Reporter Genes, pp. 258-274, Cambridge University Press, Cambridge, UK). PRGs developed to date encode proteins with various activities, including enzymes, transporters, and receptors (for review, see, e.g. Nair-Gill, E. D., et al. (2010) in Molecular Imaging with Reporter Genes (Gambhir, S. S., and Yaghoubi, S. S., eds) pp. 258-274. Cambridge University Press, Cambridge, UK). In theory, enzyme-encoding PRGs should have the highest sensitivity among different classes of PRGs as a result of signal amplification by the catalytic turnover of the enzymatic reaction that traps the probe.

The most commonly used PRGs are based on herpes simplex virus type 1 thymidine kinase (HSV1-tk) (see, e.g. Tjuvajev, J. G., et al. (1996) Cancer Res. 56, 4087-4095) and its optimized mutant, sr39tk (see, e.g. Gambhir, S. S., et al. (2000) Proc. Natl. Acad. Sci. U.S.A. 97, 2785-2790). Both wild type (WT) HSV1-tk and sr39tk have been used to study the kinetics of therapeutic cells in preclinical settings (see, e.g. Shu, C. J., et al. (2005) Proc. Natl. Acad. Sci. U.S.A. 102, 17412-17417; Yaghoubi, S. S., et al. (2007) J. Biomed. Opt. 12, 064025; Wu, J. C., et al. (2003) Circulation 108, 1302-1305; Hung, S. C., et al. (2005) Clin. Cancer Res. 11, 7749-7756). Several PRPs can be used to image cells engineered to express HSV1-tk-based PRGs: 9-[4-18F-3-(hydroxymethyl)butyl]guanine (18F-FHBG) (see, e.g. Yaghoubi, S. S., et al. (2009) Nat. Clin. Pract. Oncol. 6, 53-58; Peñuelas, I., et al. (2005) Gastroenterology 128, 1787-1795; Yaghoubi, et al. (2001) J. Nucl. Med. 42, 1225-1234), 2′-deoxy-2′-¹8F-5-ethyl-1-β-D-arabinofuranosyluracil (¹⁸F-FEAU) (see, e.g. Chin, F. T., et al. (2008) Mol. Imaging. Biol. 10, 82-91; Miyagawa, T., et al. (2008) J. Nucl. Med. 49, 637-648; Alauddin, M. M., et al. (2007) Eur. J. Nucl. Med. Mol. Imaging 34, 822-829), and 2′-deoxy-2′-¹⁸F-5-iodo-1-β-D-arabinofuranosyluracil (¹⁸F-FIAU) (see, e.g. Alauddin, M. M., et al. (2007) Eur. J. Nucl. Med. Mol. Imaging. 34, 822-829). To date, HSV1-tk is the only PRG that has been used to image therapeutic cells in patients (see, e.g. Yaghoubi, S. S., et al. (2009) Nat. Clin. Pract. Oncol. 6, 53-58).

The main disadvantage of HSV1-tk as a PRG is its immunogenicity, which can lead to immune-mediated elimination of therapeutic cells. This phenomenon has been documented in clinical trials (see, e.g. Traversari, C., et al. (2007) Blood 109, 4708-4715; Berger, C., et al. (2006) Blood 107, 2294-2302). The immunogenicity problem may be solved by replacing the viral kinase with a human orthologue (see, e.g. Amer, E. S., et al. (1995) Pharmacol. Ther. 67, 155-186). Two potentially non-immunogenic candidate PRGs based on human nucleoside kinases have been developed; that is, a double mutant of deoxycytidine kinase (dCK) (see, e.g. Likar, Y., et al. (2010) J. Nucl. Med. 51, 1395-1403) and a truncated form of mitochondrial thymidine kinase 2 (TK2) (see, e.g. Ponomarev, V., et al. (2007) J. Nucl. Med. 48, 819-826). These PRGs phosphorylate and trap the PRP ¹⁸F-FEAU. The sensitivity of the dCK-double mutant/¹⁸F-FEAU PRG-PRP system was comparable with that of HSV1-tk/¹⁸F-FEAU, whereas TK2/¹⁸F-FEAU had lower sensitivity. In non-human primates ¹⁸F-FEAU has a favorable biodistribution as a candidate PRP, with tracer accumulation in the liver, small intestine, kidneys, and urinary bladder (see, e.g. Dotti, G., et al. (2009) Mol. Imaging. 8, 230-237) but not in other organs and tissues. Human biodistribution data for this candidate PRP are not available.

The utility of a PRG-PRP system is dependent on its sensitivity (the ability to detect few therapeutic cells at various anatomical locations) and specificity (the probe should accumulate only in cells engineered to express the PRG). Another equally important parameter is the requirement that a PRG should be biologically inert. In other words its ectopic expression in therapeutic cells should not alter the metabolism or normal function of these cells. This requirement is especially important in the case of nucleoside kinase PRGs. Ectopic expression of a nucleoside kinase could perturb the normal regulation of nucleotide metabolism through excess phosphorylation of endogenous nucleosides. Such metabolic alterations can lead to imbalanced nucleotide pools and increased risk of genotoxicity (see, e.g. Kumar, D., et al. (2011) Nucleic Acids Res. 39, 1360-1371; Song, S., et al. (2003) J. Biol. Chem. 278, 43893-43896; Sargent, R. G., et al. (1987) J. Biol. Chem. 262, 5546-5553; Kumar, D., et al. (2010) Nucleic Acids Res. 38, 3975-3983). In this context the dCK-double mutant has significantly higher activity than WT dCK toward endogenous nucleosides such as deoxycytidine and thymidine (see, e.g. Hazra, S., et al. (2009) Biochemistry 48, 1256-1263). Truncated TK2 also retains normal activity with natural substrates. Whether these new PRGs fulfill the critical requirement of being biologically inert remains to be determined.

Here, the development of a new PRG-PRP system that meets the specifications mentioned above is described. The biodistribution of L-¹⁸F-FMAU, the candidate PRP, was determined in mice and humans. Enzyme engineering was used to develop a mutant PRG enzyme that is orthogonal to the wild type enzyme regarding its ability to phosphorylate endogenous nucleosides. The resulting PRG-PRP system, TK2-N93D/L109F as PRG and L-¹⁸F-FMAU as PRP, should find utility in various preclinical and clinical therapeutic cell tracking applications. The approach used to develop this system should be generalizable to the identification and evaluation of other pairs of nucleoside analogs and nucleoside kinases for PET reporter gene imaging applications.

### 6.1 Experimental Procedures.

Radiochemical Synthesis of ¹⁸F-Labeled PET Probes—

¹⁸FFHBG was synthesized as previously described (see, e.g. Yaghoubi, S., et al. (2001) J. Nucl. Med. 42, 1225-1234). The radiochemical synthesis of L-¹⁸F-FMAU is described herein (see, e.g. Example 7).

Molecular Modeling of Human TK2—

A homology model of TK2 was generated using the SWISS-MODEL server (see, e.g. Arnold, K., et al. (2006) Bioinformatics 22, 195-201). The solved structures of human dCK (35% identity, 50% homology to TK2) in both its closed (PDB ID 1P5Z) and open conformation (PDB ID 3QEO) (see, e.g. Sabini, E., et al. (2003) Nat. Struct. Biol. 10, 513-519; Hazra, S., et al. (2011) Biochemistry 50, 2870-2880) served as templates.

Generation of TK2 Mutants—

The Δ50N truncation variant of TK2 was used (which lacks the mitochondrial sorting signal), referred to as the WT enzyme. Numbering of residues is based on the full-length sequence of human TK2 (Uniprot ID O00142, see, e.g. FIG. 21). Cloning of human TK2 has been described previously (see, e.g. Hazra, S., et al. (2010) Biochemistry 49, 6784-6790). Mutants were produced on the WT TK2 sequence that was present in both the pMSCV vector for retroviral transduction and a modified pET14b expression vector for production of recombinant protein.

Expression and Purification of Recombinant TK2 Proteins—

Expression and purification of TK2 have been described previously (see, e.g. Hazra, S., et al. (2010) Biochemistry 49, 6784-6790). In short, Escherichia coli BL21 (DE3) C41 harboring the modified pET14b vector (to include a SUMO tag between the hexahistidine sequence and TK2) were grown at 37° C. until an optical density of ˜0.8 was reached. At that point the temperature was reduced to 18° C.; the culture was induced with 0.5 mM isopropyl β-D-1-thiogalactopyranoside and left to shake overnight. Cells were harvested by centrifugation, washed, and stored at −80° C. until use. Purification involved two steps. The first step used a metal affinity column (HisTRAP HP column, GE Healthcare); after elution of the His-SUMO-TK2 fusion protein, the SUMO protease was added. The cleaved protein was reapplied onto the nickel column to separate TK2 from the His-SUMO tag. The second step involved a gel filtration column (S200, GE Healthcare) equilibrated with 25 mM Tris, pH 7.5, 200 mM NaCl, and 3 mM DTT. Pure TK2 was pooled, concentrated to ˜10 mg/ml, separated into aliquots, flash-frozen in liquid nitrogen, and stored at −80° C. until use.

Kinetic Analyses of TK2-based Candidate PRGs—

A NADH-dependent enzyme coupled assay (see, e.g. Agarwal, K. C., et al. (1978) Methods Enzymol. 51, 483-490) was used. Using a Cary UV spectrophotometer, measurements were made in triplicate at 37° C. in a buffer containing 100 mM Tris, pH 7.5, 100 mM KCl, 5 mM MgCl₂, and 1 mM ATP. For data in which k_{obs} is given, a single nucleoside concentration of 200 μM was used. For data in which both Kₘ and k_{cat} are given, the nucleoside concentration was varied between 15 and 500 μM. TK2 concentration in the cuvette was 400 nM. Data were fit to the Michaelis-Menten equation using SigmaPlot. Of note, in some previous reports, negative cooperativity was observed with thymidine but not with deoxycytidine (see, e.g. Barroso, J. F., et al. (2005) Biochemistry 44, 4886-4896; Wang, L., et al. (2003) J. Biol. Chem. 278, 6963-6968). When the data for WT TK2 was fitted using the Hill equation, one also saw the same magnitude of negative cooperativity as reported by others (n=˜0.7) with thymidine and the analogs tested. However, the quality of the fit of the data is only marginally improved compared with that using the simple Michaelis-Menten equation. When the data of the TK2 mutants are fit using the Hill equation, a more complicated behavior is observed, with some conditions having a Hill coefficient below 1, some above 1, and some nearly one. Here again, the quality of the fit is not dramatically improved by adding the extra parameter of the Hill coefficient. Therefore, all of the kinetic data using the Michaelis-Menten equation without the Hill coefficient is presented.

Cell Lines—

The L1210 cell line (see, e.g. Jordheim, L. P., et al. (2004) Clin. Cancer Res. 10, 5614-5621) was a gift. Cells were cultured at 5% v/v CO₂ and 37° C. in RPMI supplemented with 5% v/v FCS. Murine stem cell virus (pMSCV)-based helper-free retroviruses encoding the TK2 mutants (or sr39tk), an internal ribosomal entry site, and the yellow fluorescent protein (YFP) were produced by transient co-transfection of the amphotrophic retrovirus packaging cell line Phoenix (American Type Culture Collection, SD 3443) (see, e.g. Hawley, R. G., et al. (1994) Gene. Ther. 1, 136-138). L1210 cells underwent spinfection with the pMSCV-TK2 mutants-internal ribosomal entry site-YFP retrovirus with 2 μg/ml Polybrene (1000×g, 120 min, 37° C.). L1210 cells expressing various PRGs, (L1210-PRG) were FACS-sorted to ensure that each population had equivalent levels of PRG expression.

Probe Uptake Assays Using Transduced L1210 Cell Lines—

L1210 cells transduced with the indicated PET reporter genes (L1210-PRG) were seeded at a density of 500,000 cells/well in 24-well plates. 5 μCi of L-¹⁸F-FMAU were added to the L1210-PRG cells simultaneously with the indicated amounts of D-thymidine (D-dT) at a final volume of 1 ml/well. After 1 h at 37° C., cells were harvested and washed four times with ice-cold PBS. Radioactivity was measured using a gamma counter.

MicroPET/CT Imaging Studies in Mice—

Animal studies were approved and carried out according to specific guidelines. C57/BL6 mice were injected with the indicated probe and underwent micro-PET/CT analyses at 1- and 3-h post probe injection (Inveon, Siemens Medical Solutions USA Inc.; microCAT; Imtek Inc.). For tumor imaging studies, SCID mice were injected subcutaneously on day −7 in the right and left flanks with 1×10⁶ L1210-PRG-expressing cells in 50% v/v phosphate-buffered saline and 50% v/v Matrigel™ (BD Biosciences). For imaging experiments, mice were kept warm and under gas anesthesia (2% v/v isoflurane) and were injected intravenously with 200 μCi of ¹⁸F-labeled probes. A 3-h interval was allowed between probe administration and microPET/CT scanning Static microPET images were acquired for 600 s. Image data were evaluated in three-dimensional histograms and reconstructed with a zoom factor of 2.1 using three-dimensional ordered set expectation maximization (OSEM) with 2 iterations followed by MAP (maximum a posteriori) reconstruction with 18 iterations (beta=0.1). Images were analyzed using OsiriX Imaging Software Version 3.8.

Human PET/CT Studies—

All studies involving human volunteers were approved. A 53-year-old healthy male and a 44-year-old healthy female volunteer were recruited for the L-¹⁸F-FMAU biodistribution study. Each volunteer received a bolus intravenous injection of ˜56 MBq (1.5 mCi) sterile L-¹⁸F-FMAU and had four consecutive whole-body (starting from just above the head to above the knees, 6 bed positions, 5-min scan at each bed position) PET scans (Biograph 64, Siemens), with the first scan starting shortly after intravenous injection of L-¹⁸F-FMAU. A low dose CT scan was also obtained for attenuation correction. Volunteers urinated after all scans had been performed. The region of interest analysis was performed to measure mean standard uptake values of L-¹⁸F-FMAU in major organs/tissues. To illustrate the biodistribution of ¹⁸F-FHBG, unpublished scan from a previous study (see, e.g. Yaghoubi, S. S., et al. (2009) Nat. Clin. Pract. Oncol. 6, 53-58) was used.

Statistical Analysis—

Data are presented as the means±S.E. All p values are two-tailed, and p values of <0.05 are considered to be statistically significant. Graphs were generated and analyzed using the Prism 5 software (GraphPad).

### 6.2 Results.

Comparison of Biodistribution of L-¹⁸F-FMAU and ¹⁸F-FHBG in Mice—

Nucleoside analogs are being increasingly used as PET probes for assaying nucleotide metabolism, cell proliferation, and mitochondrial function (see, e.g. Radu, C. G., et al. (2008) Nat. Med. 14, 783-788; Shields, A. F. (2003) J. Nucl. Med. 44, 1432-1434; Sun, H., et al. (2005) J. Nucl. Med. 46, 292-296; Mangner, T. J., et al. (2003) Nucl. Med. Biol. 30, 215-224; Namavari, M., et al. (2011) Mol. Imaging. Biol. 13, 812-818). Nucleosides can adopt one of two enantiomeric configurations. Naturally occurring nucleosides are in the D configuration (see, e.g. de Leder Kremer, R. M., et al. (2004) Adv. Carbohydr. Chem. Biochem. 59, 9-67). Recently there has been increasing interest in using nucleoside analogs with the non-natural L configuration as PET probes to image the activity of endogenous nucleoside kinases (see, e.g. Shu, C. J., et al. (2010) J. Nucl. Med. 51, 1092-1098; Nishii, R., et al. (2008) Eur. J. Nucl. Med. Mol. Imaging. 35, 990-998; Mukhopadhyay, U., et al. (2007) Appl. Radiat. Isot. 65, 941-946; Schwarzenberg, J., et al. (2011) Eur. J. Nucl. Med. Mol. Imaging. 38, 711-721). To date, L nucleosides have not been evaluated as PRPs. Determination of the potential value of L nucleosides as PRPs focused on L-¹⁸F-FMAU, the non-natural counterpart of D-¹⁸F-FMAU, one of the pyrimidine analogs that has been previously evaluated as a candidate PRP for the HSV1-tk PRG (see, e.g. Alauddin, M. M., et al. (2004) Mol. Imaging. 3, 76-84).

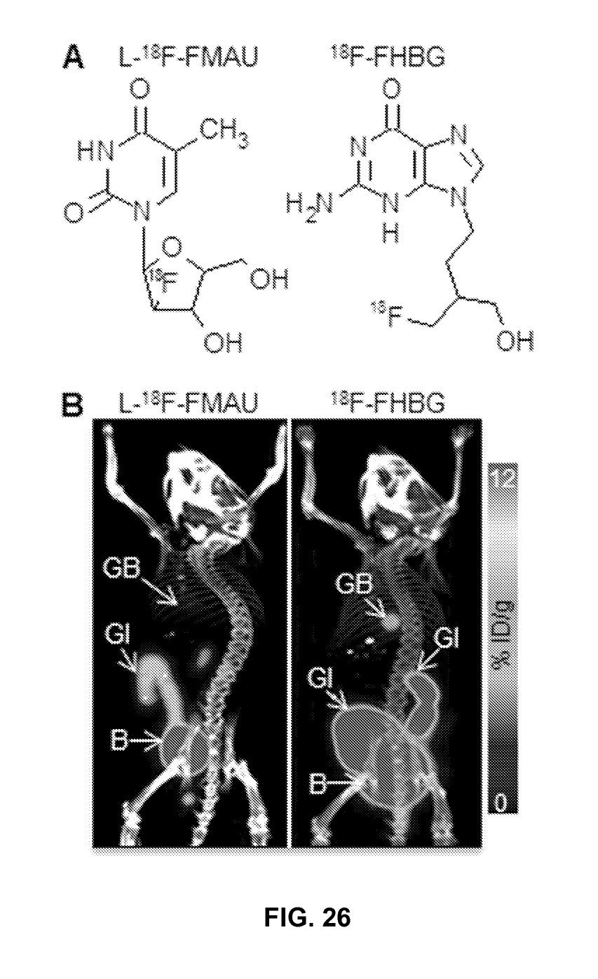

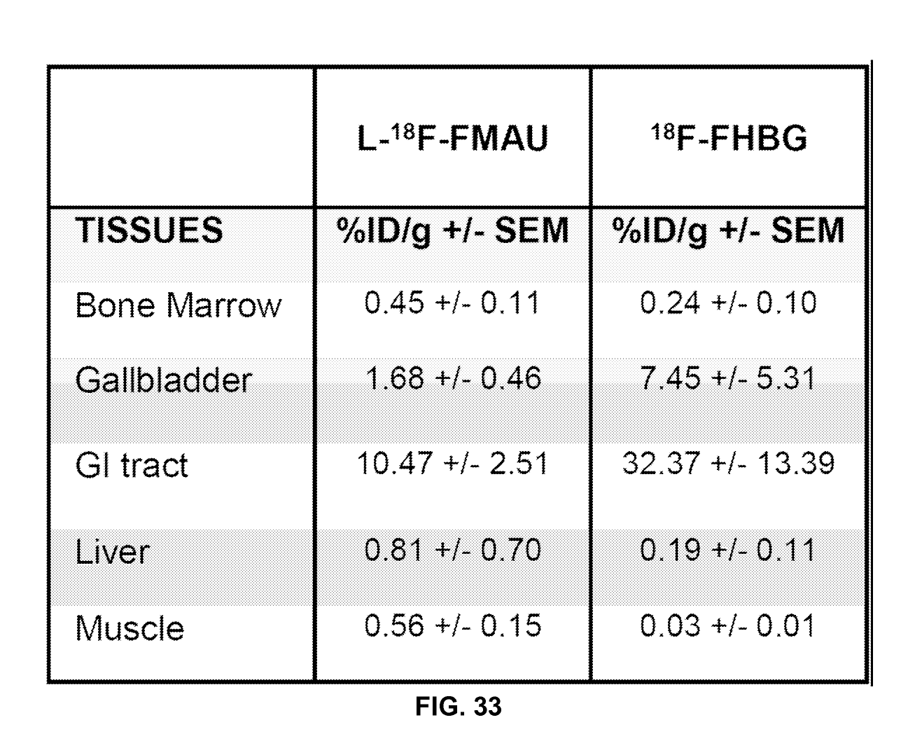

The biodistribution of L-¹⁸F-FMAU in mice was compared with that of ¹⁸F-FHBG, a well characterized and frequently used PRP (see, e.g. Yaghoubi, et al. (2001) J. Nucl. Med. 42, 1225-1234; Alauddin, M. M., et al. (1998) Nucl. Med. Biol. 25, 175-180; Alauddin, M. M., et al. (2001) J. Nucl. Med. 42, 1682-1690). To achieve optimal signal to noise ratios, PRPs should not accumulate in cells and tissues that do not express the corresponding PRG. For instance, the accumulation of the candidate PRP should be minimal or undetectable in all tissues, except in those involved in probe clearance from the body. C57/BL6 mice were scanned 3 h after administration of either L-¹⁸F-FMAU or ¹⁸F-FHBG (FIG. 26A). Three-dimensional reconstructions of the whole body microPET/CT images are shown in FIG. 1B. Quantification of the signals is presented in FIG. 33. Both L-¹⁸F-FMAU and ¹⁸F-FHBG had very low retention in the thoracic cavity. At the 3-h time point neither probe showed any accumulation in the liver. Accumulation in the gallbladder was 4 times higher for ¹⁸F-FHBG (7.45±5.31% injected dose/g) than for L-¹⁸F-FMAU (1.68±0.46% injected dose/g). Retention in the abdominal cavity was three times higher for ¹⁸F-FHBG than for L-¹⁸F-FMAU. This was likely due to higher biliary excretion of ¹⁸F-FHBG. Elevated ¹⁸F-FHBG accumulation was detected throughout the GI tract. In contrast, in mice injected with L-¹⁸F-FMAU signals were only detected in the lower GI tract. Thus, the biodistribution of L-¹⁸F-FMAU in mice was at least comparable with, if not better than that of ¹⁸F-FHBG.

Development of New PRG to be Used in Conjunction with L-¹⁸F-FMAU Candidate PRP—

L-FMAU has been shown to be a substrate for human TK2, a nucleoside kinase that due to its lack of enantiomeric specificity can phosphorylate both D and L nucleosides (see, e.g. Wang, J., et al. (1999) Biochemistry 38, 16993-16999). Ideally, modifications to the TK2 sequence should achieve two objectives; (i) increase sensitivity by reducing the negative feedback regulation of the enzyme and by increasing the phosphorylation rate of the L-FMAU PRP; (ii) reduce the activity of the PRG kinase for the endogenous substrates thymidine and deoxycytidine (to avoid competition between L-FMAU and endogenous nucleosides and potentially genotoxic perturbations of endogenous nucleotide pools).

The enzymatic activity of TK2 is regulated by thymidine triphosphate (dTTP) through negative feedback inhibition (see, e.g. Radivoyevitch, T., et al. (2011) Nucleosides Nucleotides Nucleic Acids 30, 203-209). dTTP is produced by de novo synthesis and through the salvage of thymidine (via the cytosolic nucleoside kinase TK1). dTTP levels fluctuate throughout the cell cycle and are highest during the S phase, when they increase by as much as 2.5-20-fold compared with the G1 phase (see, e.g. Bianchi, V., et al. (1997) J. Biol. Chem. 272, 16118-16124; Spyrou, G., et al. (1988) Mutat. Res. 200, 37-43). It is possible that fluctuations in dTTP levels during the cell cycle can reduce sensitivity and result in difficult to interpret changes in PET signals.

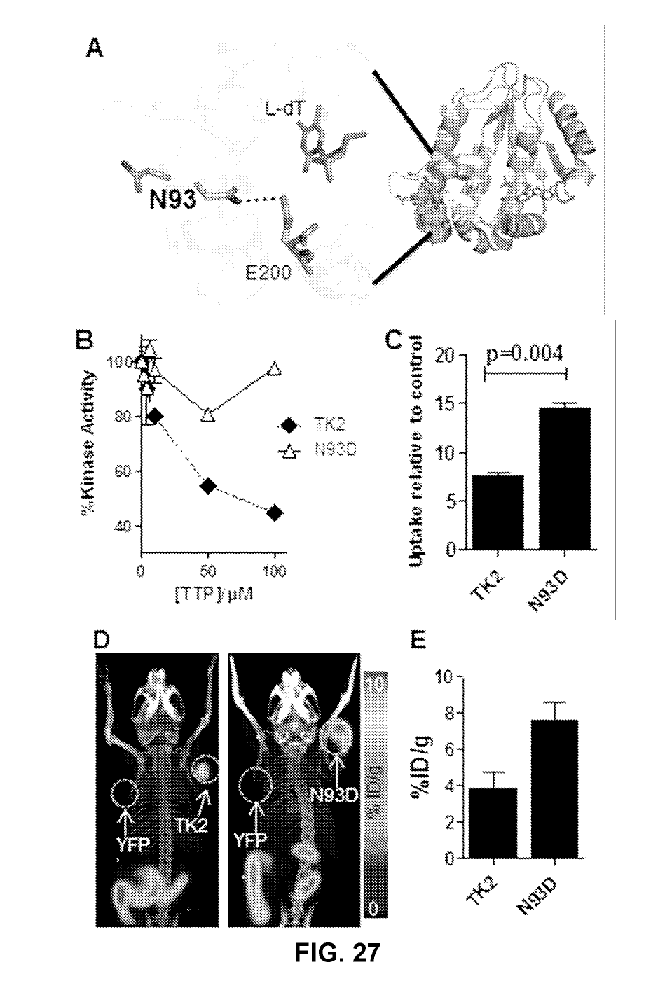

To reduce the susceptibility of TK2 to dTTP-mediated feedback inhibition, one took advantage of the 40% sequence identity between human TK2 and Drosophila melanogaster deoxyribonucleoside kinase (Dm-DNK) (see, e.g. Eriksson, S., et al. (2002) Cell. Mol. Life. Sci. 59, 1327-1346) and of the identification of a point mutation (N64D) in Dm-DNK that has been shown to reduce the effect of dTTP feedback inhibition (see, e.g. Welin, M., et al. (2005) FEBS J. 272, 3733-3742). The residue in TK2 corresponding to Asn-64 in D. melanogaster deoxyribonucleoside kinase is Asn-93; the corresponding mutation in TK2 is N93D. To predict the effects of the N93D mutation on the structure of TK2, molecular modeling was used. One took advantage of the fact that dCK belongs to the same family of nucleoside kinases as TK2. The sequence identity and homology between dCK and TK2 are 35 and 50%, respectively. Based on previous works with dCK (see, e.g. Sabini, E., et al. (2003) Nat. Struct. Biol. 10, 513-519; Hazra, S., et al. (2011) Biochemistry 50, 2870-2880), a homology model of TK2 was obtained (FIG. 27A). It was hypothesized that, similar to dCK, TK2 can also adopt an open or a closed conformation. The enzyme is expected to be active in the closed conformation and inactive in the open conformation. In the model, when TK2 is in the closed conformation, Asn-93 is involved in hydrogen bonding with the glutamine at position 200 (E200, FIG. 27A). When the enzyme is in the open conformation, the residues are too far apart to interact. Thus, the N93D mutation would be expected to disfavor the closed conformation due to disruption of the interaction between Asn-93 and Glu-200 (FIG. 27A). dTTP should be able to exert its negative feedback inhibition on TK2 only if the enzyme is in the closed conformation. Because the N93D mutation favors the open conformation of the enzyme, it was predicted there would be a reduced probability for dTTP to bind and exert its inhibitory effect.

To test this hypothesis, kinase assays using L-FMAU and recombinant WT TK2 and TK2-N93D were performed in the presence of varying amounts of dTTP (FIG. 27B).WTTK2 activity decreased by 20% in the presence of 10 μM dTTP. In contrast, the activity of the N93D mutant decreased by only 4%. When the dTTP concentration was increased to 100 μM, the activity of WT TK2 decreased by 55%, whereas that of TK2-N93D decreased by less than 5%.

Cell-based uptake assays were then used to determine whether the decreased susceptibility to feedback inhibition conferred by the N93D mutation increases L-¹⁸F-FMAU uptake. As shown in FIG. 27C, a 1.5-fold increase was observed in L-¹⁸F-FMAU uptake by the N93D TK2 expressing L1210 cells relative to cells expressing similar levels of WT TK2.

To confirm that the increase in signal can also be detected in vivo, mice implanted with L1210 cells transduced with the WT TK2 and mutant TK2-N93D PRGs (FIG. 27D) were used. In vivo, L-¹⁸F-FMAU uptake by TK2-N93D PRG-expressing cells was nearly double of that observed with the WT TK2-expressing cells (FIGS. 27, D and E). Thus, by engineering a TK2 mutant that is less sensitive to feedback inhibition, one was able to improve the sensitivity of this candidate PRG for L-¹⁸F-FMAU.

Further Improvements of Selectivity and Affinity of TK2-Derived PRG for L-¹⁸F-FMAU—

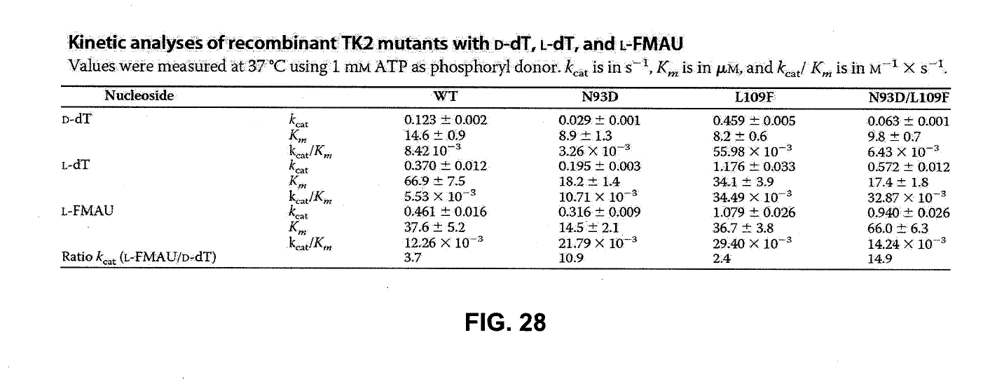

For enzymatic PRGs, the higher the catalytic turnover (k_{cat}) of the enzyme, the more the PRP can accumulate per unit time, leading to a higher PET signal. The k_{cat} of mutated TK2 PRG for L analogs was determined compared with the endogenous substrate, D-dT. Relative to WT TK2, the N93D mutation reduced the k_{cat} of the enzyme toward D-dT, L-dT, and L-FMAU (FIG. 28). However, the activity toward D-dT decreased by 77%, whereas that for L-dT and L-FMAU decreased only 48 and 32%, respectively. The k_{cat} (L-FMAU)/k_{cat} (D-dT) ratio for N93D nearly triples when compared with wild type. k_{cat}/Kₘ gives a measure of the substrate preference of an enzyme. Compared with WTTK2, the k_{cat}/Kₘ of N93D for L-FMAU increased by 77%, whereas the k_{cat}/Kₘ for D-dT decreased by 60%. Thus, the N93D mutation also achieved the goal of increasing the preference of the enzyme for L-FMAU over the natural substrate.

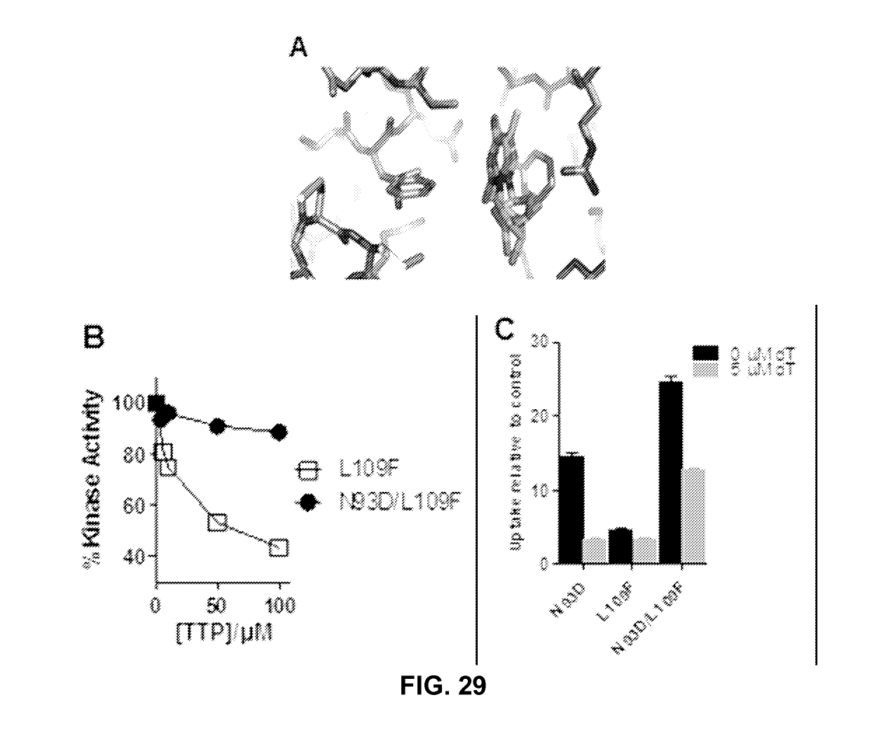

To identify additional mutations that may further improve the selectivity of the TK2 PRG for L analogs, high resolution structures of dCK in complex with L and D substrates (see, e.g. Sabini, E., et al. (2007) J. Med. Chem. 50, 3004-3014) were used to generate a homology model of TK2 with bound L-dT and D-dT (FIG. 29A). This model was then used to identify residues that, when mutated, would result in an enzyme with increased affinity for L-dT and decreased affinity for D-dT. This approach led to the identification of residue Leu-109 (FIG. 29A). According to the homology model, this residue interacts with the pyrimidine base. It is surmised that if Leu-109 were mutated to an amino acid with a bulkier side chain (e.g. phenylalanine), this would induce a steric clash with D nucleosides but less so with L nucleosides. In turn, this would lead to preferential binding of L versus D nucleosides. Contrary to one's expectations, the L109F mutation led to a decrease in the Kₘ for both the D and L forms of dT (FIG. 28). Notably, the L109F mutation made the enzyme faster at phosphorylating all of the substrates tested, with a bigger effect on D-dT. Thus, for WT TK2, the k_{cat} (L-FMAU)/k_{cat} (D-dT) ratio is 3.7, whereas for TK2-L109F this is 2.4 (FIG. 28). Compared with WTTK2, the k_{cat}/Kₘ of L109F for L-FMAU increased 2.4 times, whereas the k_{cat}/Kₘ for D-dT increased 6.7 times. Thus, contrary to the prediction, the L109F mutation increased the preference of the enzyme for D-dT compared with L-FMAU. This demonstrates that although a homology model can be sufficient to identify “hot spots” for mutagenesis (in this case, position 109), such a model may lack accuracy that can only be attained by an experimentally derived model. Nevertheless, although the L109F did not provide the desired increase in selectivity toward L nucleosides, it is important to note that the L109F mutation did increase the overall speed of the enzyme for all tested substrates.

Based on these observations, TK2-N93D/L109F was generated with the expectation that this double mutant will combine the enzymatic properties of the two single mutants. As shown in FIG. 28, this was indeed the case. Compared with TK2, the N93D/L109F double mutant had decreased k_{cat} with D-dT (down 49%) but increased k_{cat} with L-dT (up 54%) and L-FMAU (up 100%). The k_{cat} (L-FMAU)/k_{cat} (D-dT) ratio for the TK2-N93D/L109F mutant is 14.9, 4-fold higher than that for TK2 and nearly 40% higher than that for TK2-N93D. Importantly, the TK2-N93D/L109F mutant still retained resistance to inhibition by dTTP (FIG. 29B). In the presence of 10 μM dTTP, the kinase activity of recombinant TK2-N93D/L109F decreased by only 4%, whereas that of TK2-L109F decreased by 25%. At 100 μM dTTP, TK2-N93D/L109F decreased by only 11%, whereas TK2-L109F decreased by 56%.

To determine the preference of the TK2 mutants for L-¹⁸F-FMAU over D-dT, uptake assays were performed using L1210 cells in the presence or absence of 5 μM D-dT (FIG. 29C). L-¹⁸F-FMAU uptake by TK2-N93D/L109F-expressing L1210 cells in the absence of D-dT was 1.5 times higher than that of TK2-N93D cells and nearly 4 times higher than that of TK2-L109F cells. In the presence of 5 μM D-dT, L-¹⁸F-FMAU uptake by TK2-N93D/L109F cells decreased by 47%, whereas that of TK2-N93D cells decreased by 75%. Although the L-¹⁸F-FMAU uptake of TK2-L109F in the presence of 5 μM D-dT decreased by 37%, it was still only 31% of the corresponding uptake for TK2-N93D/L109F.

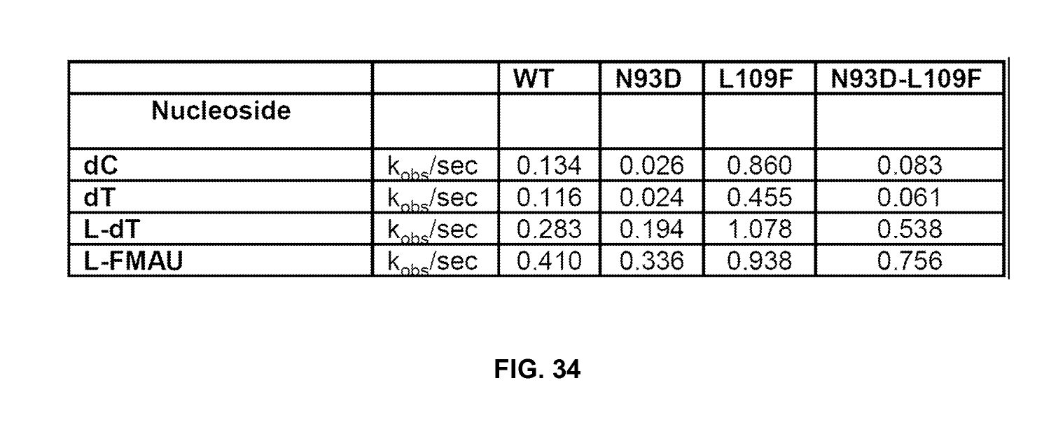

Next, one investigated whether TK2-N93D/L109F had low activity toward deoxycytidine, the other endogenous nucleoside that is phosphorylated by WTTK2. TK2-N93D/L109F has a k_{obs} (dC) that is 62% that of TK2 (FIG. 34). These data indicate that TK2-N93D/L109F is orthogonal to wild type TK2, with increased activity toward the L-¹⁸F-FMAU PRP and decreased activity toward the endogenous nucleosides thymidine and deoxycytidine.

In Vivo Comparison Between TK2-N93D/L109F/L-¹⁸FFMAU and HSV1-sr39tk/¹⁸F-FHBG PRG-PRP Systems—

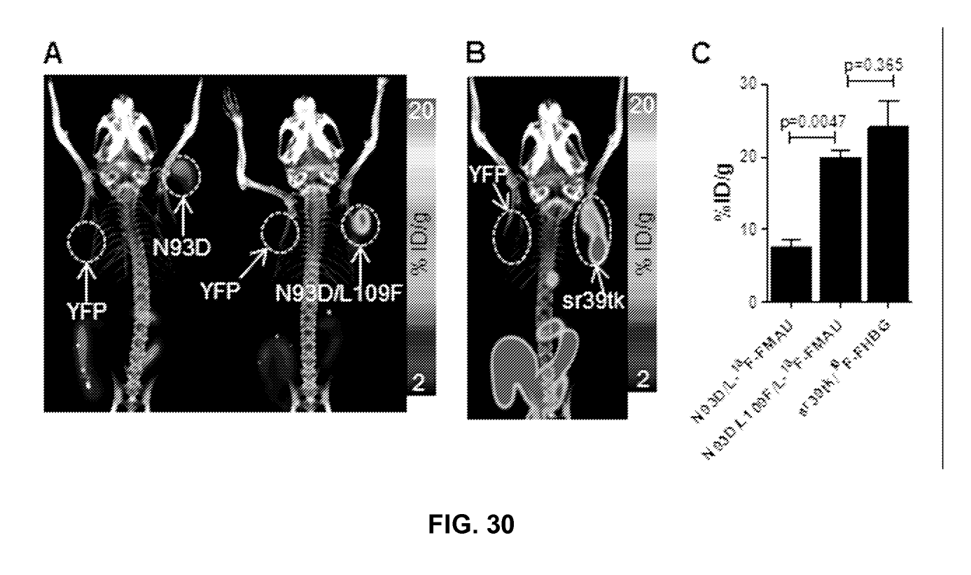

Mice implanted with L1210 cells expressing TK2-based PRGs were scanned by microPET/CT using L-¹⁸F-FMAU (FIG. 30A). For comparison, mice implanted with L1210 cells expressing HSV1-sr39tk were scanned by microPET/CT using ¹⁸F-FHBG (FIG. 30B). L-¹⁸F-FMAU uptake by the TK2-N93D/L109F-expressing L1210 cells was 2.6-fold higher than that of TK2-N93D-expressing cells (FIG. 30C). ¹⁸F-FHBG accumulation into sr39tk-expressing L1210 cells was comparable with that of L-¹⁸F-FMAU into L1210 cells expressing TK2-N93D/L109F (24.1±6.2 versus 19.9±1.5% injected dose/g; p=0.37). Taken together, these findings demonstrate that the sensitivity of the TK2 N93D/L109F PRG is higher than that of the TK2-N93D PRG and is not significantly different from that of the sr39tk/¹⁸F-FHBG pair.

L-¹⁸F-FMAU Biodistribution in Humans—

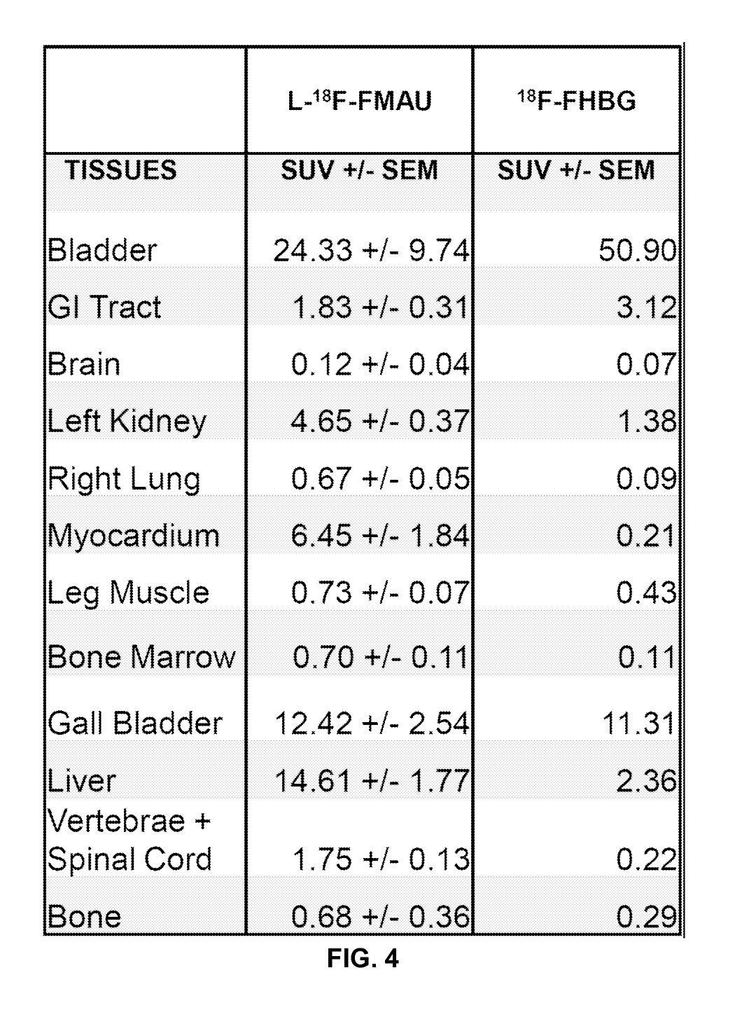

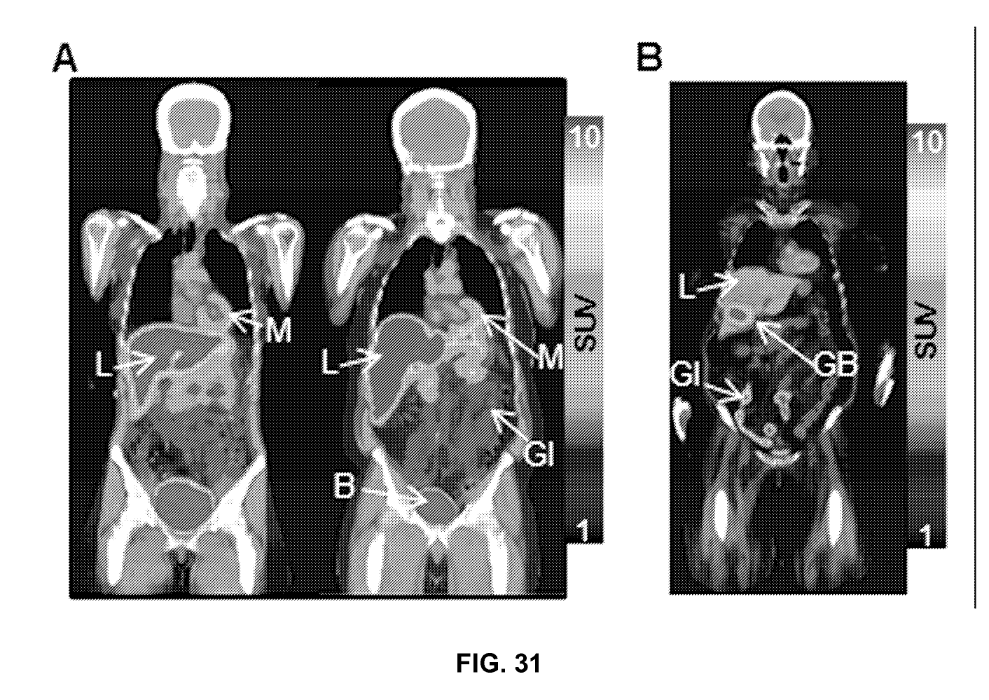

As the first step toward clinical translation of the newly developed PRG-PRP system, the biodistribution of L-¹⁸F-FMAU in humans was determined. FIG. 31 illustrates the biodistribution of L-¹⁸F-FMAU in two healthy volunteers and the biodistribution of ¹⁸F-FHBG in a female volunteer 2 h post-administration of the PRPs. Mean standard uptake values of the probes in different tissues for ¹⁸F-FHBG and L-¹⁸F-FMAU are listed in FIG. 4. For both probes, relatively high signals were observed in liver, kidneys, gall bladder, bladder, and the GI tract. L-¹⁸F-FMAU accumulation was also observed in the myocardium. At 2 h, more intense activity was observed in the liver after L-¹⁸F-FMAU injections than after ¹⁸F-FHBG administration. However, L-¹⁸F-FMAU activity was lower than that of ¹⁸F-FHBG within the GI tract region.

### 6.3 Discussion.

To develop a PRG that can be used in conjunction with L-¹⁸F-FMAU, a thymidine analog with the unnatural L-conformation, the mitochondrial sorting sequence was removed in human TK2. As shown previously, the truncated protein is expected to localize in the cytosol rather than in the mitochondria (see, e.g. Ponomarev, V., et al. (2007) J. Nucl. Med. 48, 819-826). Rational design was then used to improve the sensitivity and selectivity of the TK2 PRG. This led to the development of TK2-N93D/L109F, a double mutant TK2 kinase characterized by reduced affinity for the natural substrates D-thymidine and D-deoxycytidine and increased affinity for L-FMAU. Studies in mice indicated that the TK2-N93D/L109F PRG has comparable sensitivity to that of the widely used HSV1-sr39tk/¹⁸F-FHBG system. The biodistribution of L-FMAU in humans has also been determined.

Advantages of TK2-N93D/L109F/L-¹⁸F-FMAU PRG System—

In mice, L-¹⁸F-FMAU accumulates in the liver 1-h post injection (data not shown). The progression of the signal from the liver to the gallbladder and then to the GI indicates that L-¹⁸F-FMAU is excreted via a hepato-biliary mechanism, similar to that observed for ¹⁸F-FHBG (see, e.g. Yaghoubi, et al. (2001) J. Nucl. Med. 42, 1225-1234). However, the GI activity in L-¹⁸F-FMAU-injected mice is significantly less intense than that observed in mice injected with ¹⁸F-FHBG. The intense signal in the GI of mice injected with ¹⁸F-FHBG leads to spillover in other organs in the lower abdomen, limiting the utility of ¹⁸F-FHBG for cell tracking applications in mice if these cells localize in the abdominal cavity.

In addition to its human origin (which is expected to reduce immunogenicity compared with the viral PRGs), the TK2-N93D/L109F PRG also has the advantage of reduced activity toward the endogenous nucleosides, D-thymidine and D-deoxycytidine. PRGs are typically overexpressed in therapeutic cells. In this context, if the mutant PRG retains the ability to efficiently phosphorylate thymidine and/or deoxycytidine, then this may alter cellular metabolism due to overproduction of dTTP and/or dCTP. Such effects would be of particular concern in preclinical settings as serum levels of thymidine in mice and rats are 9-15 times higher than those in humans (see, e.g. Nottebrock, H., et al. (1977) Biochem. Pharmacol. 26, 2175-2179). Any changes in nucleotide metabolism and dNTP pools in therapeutic cells may have genotoxic consequences, especially when prolonged persistence in vivo of these cells is anticipated (for example in the case of stem cells). In contrast to previously reported PRG such as dCK-double mutant, TK2-N93D/L109F is less likely to perturb cellular nucleotide metabolism and genomic integrity due to the decreased activity of the double mutant enzyme toward natural substrates.

In contrast to mice, in humans L-¹⁸F-FMAU accumulates in the myocardium and liver. Regarding L-¹⁸F-FMAU accumu-mitochondria (see, e.g. Schaper, J., et al. (1985) Circ. Res. 56, 377-391). Moreover, the reported activity of the WT mitochondrial TK2 enzyme from human heart tissue is nearly 10 times higher than that of the enzyme from mouse heart tissue (see, e.g. Saada, A., et al. (2003) Mol. Genet. Metab. 79, 1-5; Wang, L., et al. (2000) Biochem. J. 351, 469-476). This difference may explain the observed differences in L-¹⁸F-FMAU myocardial accumulation between mice and humans. L-¹⁸F-FMAU is taken up by the liver of both mice and humans. However, L-¹⁸F-FMAU eventually clears the murine liver but is retained in the human liver. One reason for this difference may be that, similar to ¹⁸F-FLT (3′-deoxy-3′-¹⁸-fluorothymidine) (see, e.g. Shields, A. F., et al. (1998) Nat. Med. 4, 1334-1336), L-¹⁸F-FMAU may also undergo glucuronidation in human liver tissue. Glucuronidation of thymidine analogs is significantly less extensive in mice than in humans (see, e.g. Barthel, H., et al. (2003) Cancer Res. 63, 3791-3798). Myocardial accumulation in humans may be reduced if L-¹⁸F-FMAU is modified to decrease its phosphorylation by WT TK2. Replacing the 5-methyl group with a larger substituent such as ethyl or propyl may achieve this objective.

As disclosed herein, a structure guided approach was used to develop a human nucleoside kinase-based PRG characterized by high specificity and selectivity for L-¹⁸F-FMAU, a non-natural nucleoside analog PRP. The initial findings in mice and the observed biodistribution of L-¹⁸F-FMAU in mice and humans warrant additional studies in both species and provide evidence for strategies to further improve the sensitivity and specificity of the new human TK2-based PET reporter gene assay.

### Example 7

### Illustrative Synthesis of L-¹⁸F-FMAU

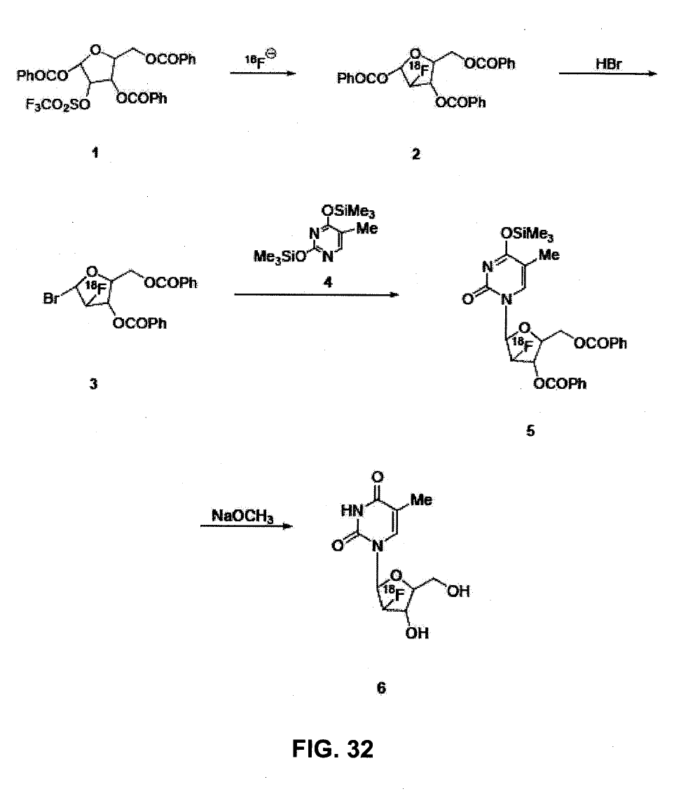

As shown in FIG. 32, L-2-O-[(Trifluoromethyl)sulfonyl]-1,3,5-tri-O-benzoyl-α-D-ribofuranose 1 was prepared based on the procedure reported for the corresponding D-isomer (see, e.g. Tann, C. H., et al. J. Org. Chem. 1985, 50, 3644-3647). The synthesis of the ¹⁸F-fluoro analog 2 was carried out by a modification of the method reported by Mangner et al (see, e.g. Mangner, T. J., et al. Nuc. Med. Biol. 2003, 50, 215-224) for the corresponding D-isomer. No-carrier-added [¹⁸F]-fluoride ion was produced by 11 MeV proton bombardment of 98% enriched [¹⁸O]water in a tantalum target body using a RDS-112 cyclotron. The aqueous [¹⁸F]fluoride ion was passed through a small cartridge of BioRad MP-1 anion exchange resin (10 mg, bicarbonate form) to trap the [¹⁸F]fluoride ion. The [¹⁸F]fluoride ion was subsequently released from the cartridge with a solution of K₂CO₃ (1 mg in 0.4 mL of water) and mixed with a solution of Kryptofix 2.2.2 (10 mg) dissolved in water (0.04 mL) and acetonitrile (0.75 mL) mixture.

The solution was evaporated at 115° C. with a stream of nitrogen gas. The residue was dried by the azeotropic distillation with acetonitrile (3×0.5 mL). To the dry residue, a solution of the triflate 1 (10 mg) in 0.7 mL of acetonitrile was added and the reaction mixture was heated at 165° C. for 15 min in a sealed vessel. The solution was cooled to room temperature and passed through a Waters silica gel Sep-Pak. The product was eluted from the cartridge with 5 mL of ethyl acetate. The ethyl acetate solution was evaporated to dryness and 0.1 mL of a solution of 30% HBr in acetic acid was added followed by 0.4 mL of dichloroethane.

This new reaction mixture was heated at 80° C. in a sealed vessel for 10 min and the solution was concentrated to ˜50% of the initial volume. Toluene (0.7 mL) was then added and the solution was evaporated at 110° C. to give the bromo compound 3. A solution of the disilyl derivative of 5-methyluracil (4, 20 mg, Aldrich Chemical Company) was dissolved in 1 mL of dichloroethane and added to the bromo compound 3. The condensation reaction was carried out at 160° C. in a sealed vessel for 30 min. The reaction mixture was cooled to room temperature and then passed through a Waters silica gel Sep-Pak.

The product was eluted off the column using 5 mL of a solution mixture of 10% methanol and 90% dichloromethane. This solution was evaporated to dryness at 100° C. and then treated with 0.5 mL of a solution of 0.5 M sodium methoxide in methanol. The reaction mixture was heated at 100° C. for 5 min in a sealed vessel. The basic reaction mixture was neutralized with 0.25 mL of 1M HCl in water. This reaction mixture was diluted to a total volume of 3 mL with a mixture of 4% acetonitrile and 96% 50 mM ammonium acetate in water and injected into a semi-preparative HPLC column (Phenomenex Gemini C-18 column; 25 cm×1 cm). The HPLC column was eluted with a solvent mixture of 4% acetonitrile and 96% 50 mM ammonium acetate at a flow rate of 5.0 mL/min. The effluent from the HPLC column was monitored with a 254 nm UV detector followed by a gamma radioactive detector. The chemically and radiochemically pure L-[¹⁸F] FMAU (6) that eluted off the column with a retention time of ˜24 min was collected and the solvents were evaporated in a rotary evaporator. One mL of ethanol was added to the residue and evaporated to remove the last traces of acetonitrile. This was followed by an addition of one mL sterile water and evaporation to remove the ethanol. The product was finally dissolved in 5 mL of sterile water and made isotonic with saline and sterilized by passing through a Millipore filter (0.22 μm) into a sterile multi-dose vial.

### Example 8

### Illustrative PET Reporter Gene Probes

As number of illustrative PRPs are disclosed including FFU, FCU, FBU and FddUrd (FIG. 25). The ideal PET reporter gene probe should satisfy all of the following 4 conditions: a) the probe should not be a substrate for wild type thymidine kinase 1 (TK1); b) the probe should be amenable to F-18 labeling, in a minimal number of steps (ideally 2) and with high specific activity; c) the probe should have good biodistribution in mice and humans (i.e., the probe should be able to gain access to tissues, but should not accumulate by specific or non-specific mechanisms in any tissue); and d) the probe should be metabolically stable in vivo.

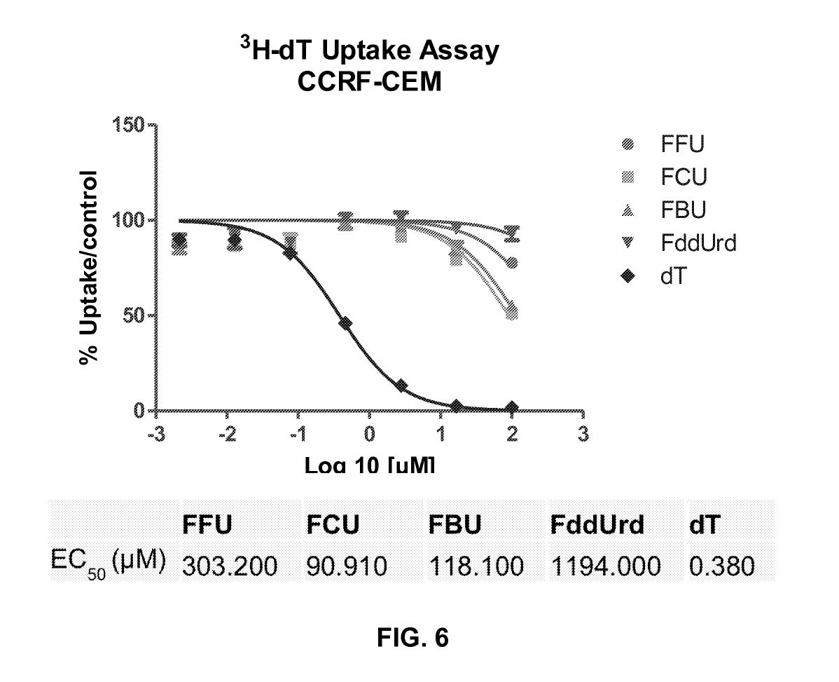

As shown in FIG. 6, all four probes satisfy condition (a). FddUrd may have the edge over the other 3 candidate probes in an [³H]dT uptake inhibition assay. However, this particular assay does not distinguish between competition at the level of transport and competition at the level of phosphorylation by TK1. Further evaluations can be carried out to precisely assess the affinity of these candidate probes for purified recombinant TK1.

All four probes satisfy condition (b). One has been able to synthesize all of them in amounts that were more than sufficient for animal studies.

Representative images of the biodistribution of the 4 candidate probes in mice are shown in FIG. 25B. Essentially all 4 probes display excellent biodistribution in immune competent (C57/BL6) mice. Thus, these probes satisfy condition (c) in mice.

Regarding condition (d), one has obtained the microsomal stability profiles for 3 out of 4 compounds (FCU, FBU and FddUrd using the glucuronidation conditions). Of these three candidate probes, only FBU showed decrease stability over time in the microsomal assay (data not shown).

FCU has previously been evaluated in humans for its anti-HIV properties. The existence of a toxicology study in humans can allow for us to get initial biodistribution studies of FCU in humans.

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

Certain aspects of the invention are disclosed in Campbell et al., J Biol. Chem. 2012 287(1):446-54, the contents of which are incorporated by reference. All publications, patents, and patent applications cited herein are hereby incorporated by reference in their entirety for all purposes.

1. A human thymidine kinase polypeptide comprising amino acid residues 51-265 of SEQ ID NO: 1, wherein:
the thymidine kinase polypeptide comprises an amino acid substitution at amino acid residue position 93 and/or amino acid residue position 109 of SEQ ID NO: 1; and
the thymidine kinase polypeptide can phosphorylate 2′-deoxy-2′-18^{F}-5-methyl-1-β-L-arabinofuranosyluracil.

2. The thymidine kinase polypeptide of claim 1, wherein the polypeptide comprises an amino acid substitution at amino acid residue position 93 of SEQ ID NO: 1.

3. The thymidine kinase polypeptide of claim 2, wherein the amino acid substitution comprises N93D.

4. The thymidine kinase polypeptide of claim 1, wherein the polypeptide comprises an amino acid substitution at amino acid residue position 109 of SEQ ID NO: 1.

5. The thymidine kinase polypeptide of claim 4, wherein the amino acid substitution comprises L109M or L109F.

6. The thymidine kinase polypeptide of claim 1, wherein the polypeptide comprises an amino acid substitution at amino acid residue position 93 and an amino acid substitution at amino acid residue position 109 of SEQ ID NO: 1.

7. The thymidine kinase polypeptide of claim 6, wherein the polypeptide comprises a set of amino acid substitutions comprising N93D/L109M or N93D/L109F.

8. A nucleic acid molecule comprising DNA encoding the human thymidine kinase polypeptide of claim 1.

9. A vector comprising the nucleic acid molecule of claim 8.

10. The vector of claim 9 operably linked to control sequences recognized by a host cell transfected with the vector.

11. A system for imaging a mammalian cell using positron emission tomography (PET) or single photon emission computed tomography (SPECT), the system comprising a PET reporter gene and a PET reporter probe, wherein:
the PET reporter gene encodes a human thymidine kinase;
the PET reporter probe comprises a non-naturally occurring analog of thymidine; and
a polypeptide encoded by the PET reporter gene phosphorylates the non-naturally occurring analog of thymidine.

12. The system of claim 11, wherein the PET reporter probe is selected from the group consisting of: L-[18^{F}]FMAU, L-[18^{F}]FEAU, L-[18^{F}]FBrVAU, L-[18^{F}]FBU, D-[18^{F}]FMAU, D-[18^{F}]FEAU, D-[18^{F}]FBrVAU, D-[18^{F}]FCU, [18^{F}]FHBG, FFU and FddUrd.

13. The system of claim 11, wherein the PET reporter gene encodes a human thymidine kinase 2 polypeptide (Uniprot ID O00142).

14. The system of claim 11, wherein the PET reporter gene encodes a thymidine kinase 2 polypeptide comprising amino acid residues 51-265 of SEQ ID NO: 1, wherein the thymidine kinase polypeptide comprises a deletion mutation or a substitution mutation that confers a decreased susceptibility to thymidine triphosphate mediated feedback inhibition as compared to wild type SEQ ID NO: 1.

15. The system of claim 11, wherein the PET reporter gene encodes a thymidine kinase polypeptide comprising amino acid residues 51-265 of SEQ ID NO: 1, wherein the thymidine kinase polypeptide comprises an amino acid substitution at amino acid residue position 93 and/or amino acid residue position 109 of SEQ ID NO: 1.

16. The system of claim 15, wherein the thymidine kinase polypeptide comprises a set of amino acid substitutions comprising N93D/L109M or N93D/L109F.

17. The system of claim 11, wherein:
the PET reporter gene encodes a thymidine kinase polypeptide comprising amino acid residues 51-265 of SEQ ID NO: 1, and
the PET reporter gene encodes a thymidine kinase polypeptide having at least one insertion, substitution or deletion mutation in SEQ ID NO: 1.

18. The system of claim 11, wherein the PET reporter probe and/or the PET reporter gene is combined with a pharmaceutically acceptable carrier.

19. The system of claim 11, wherein the system is disposed in a kit, the kit comprising:
a first container comprising a vector that comprises the PET reporter gene, wherein the PET reporter gene is covalently coupled to vector control sequences recognized by a host cell transformed with the vector; and
a second container comprising the PET reporter probe.

20. A method of imaging a mammalian cell using positron emission tomography (PET) or single photon emission computed tomography (SPECT), the method comprising the steps of:
a) introducing a reporter gene into a mammalian cell, the reporter gene encoding a thymidine kinase polypeptide comprising amino acid residues 51-265 of SEQ ID NO: 1;
b) introducing a reporter probe comprising a non-naturally occurring analog of thymidine, wherein the thymidine kinase polypeptide encoded by the reporter gene is able to phosphorylate the non-naturally occurring analog of thymidine; and
c) detecting the reporter probe using positron emission tomography (PET) or single photon emission computed tomography (SPECT).

21. The method of claim 20, wherein the reporter probe is selected from the group consisting of: L-[18^{F}]FMAU, L-[18^{F}]FEAU, L-[18^{F}]FBrVAU, L-[18^{F}]FBU, D-[18^{F}]FMAU, D-[18^{F}]FEAU, D-[18^{F}]FBrVAU, D-[18^{F}]FCU, [18^{F}]FHBG, FFU and FddUrd.

22. The method of claim 20, wherein the thymidine kinase polypeptide:
consists essentially of amino acid residues 51-265 of SEQ ID NO: 1; and
comprises at least one of an amino acid substitution at amino acid residue position 93 or amino acid residue position 109 of SEQ ID NO: 1.

23. The method of claim 22, wherein the amino acid substitution comprises at least one of an N93D, L109M or L109F amino acid substitution in SEQ ID NO: 1.

24. The method of claim 22, wherein the thymidine kinase polypeptide comprises a set of amino acid substitutions comprising N93D/L109M or N93D/L109F.

25. The method of claim 20, wherein the reporter gene is introduced to the mammalian cell by transfecting the mammalian cell with a vector comprising a nucleic acid molecule encoding the thymidine kinase polypeptide and wherein the vector is operably linked to control sequences recognized by the mammalian cell transfected with the vector.
